(19) United States
(12) Patent Application Publication
(10) Pub. No.: US 2026/0193267 A1
(43) Pub. Date: Jul. 9, 2026

(54) Solid Forms of Naphthyridine Compounds

(71) Applicant: AMGEN INC., Thousand Oaks, CA (US)

(72) Inventors: Weikun Li, Thousand Oaks, CA (US); Hyunsoo Park, Boston, MA (US); Darren Leonard Reid, Belmont, MA (US); Adam Nicholas Smith, Cambridge, MA (US); Tian Wu, Newbury Park, CA (US)

(21) Appl. No.: 19/134,236

(22) PCT Filed: Dec. 5, 2023
(86) PCT No.: PCT/US2023/082448

§ 371 Date: May 30, 2025

(51) Int. Cl. C07D 491/147; A61K 31/5377

(52) U.S. Cl. CPC C07D 491/147; A61K 31/5377

(57) ABSTRACT

Disclosed herein are various solid forms of Compound A, including crystalline and amorphous forms of Compound A free base, as well as salt forms, cocrystals, and solvates thereof. Also disclosed are methods of making the salt, cocrystal, and solvate forms, and methods of treating diseases and disorders with the salt, cocrystal, and solvate forms (Compound A).

### BACKGROUND

Naphthyridine compounds have been shown to be important in a number of biological applications. In order to investigate their efficacy, large quantities of the materials are needed. As such, there is a need for solid forms of naphthyridine compounds, including crystalline forms, amorphous forms, salts, cocrystals and solvates thereof and processes for the isolation thereof.

### SUMMARY

The disclosure provides solid forms of Compound A, including crystalline forms, amorphous forms, as well as salts, solvates, and cocrystals thereof, wherein Compound A has the structure

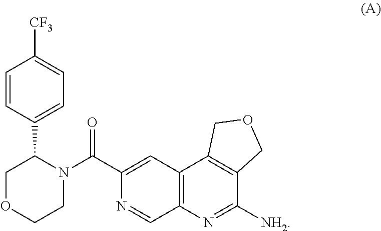 [SMILES: Nc1nc2cnc(C(=O)N3CCOC[C@@H]3c3ccc(C(F)(F)F)cc3)cc2c2c1COC2]

In some embodiments, the disclosure provides an amorphous form of Compound A free base.

In some embodiments, the disclosure provides a crystalline form of Compound A.

In some embodiments, the disclosure provides crystalline Compound A in free base form.

In some embodiments, the disclosure provides crystalline forms of Compound A free base (“Form 1”), characterized by an X-ray powder diffraction (XRPD) pattern comprising peaks at 4.5, 9.0, 13.3, 16.2, and 18.8±0.2° 2θ using CuKα radiation.

In some embodiments, the disclosure provides crystalline forms of Compound A free base (“Form 3”), characterized by an XRPD pattern comprising peaks at 4.3, 12.6, 14.4, 16.2, and 25.6±0.2° 2θ using CuKα radiation.

In some embodiments, the disclosure provides crystalline forms of Compound A free base (“Form 6”), characterized by an XRPD pattern comprising peaks 4.5, 8.6, 9.0, 12.9, and 14.9±0.2° 2θ using CuKα radiation.

In some embodiments, the disclosure provides crystalline forms of Compound A free base (“Form 7”), characterized by an XRPD pattern comprising peaks 4.3, 8.5, 12.3, 13.1, and 14.8±0.2° 2θ using CuKα radiation.

In some embodiments, the disclosure provides crystalline forms of Compound A free base hydrate (“Form 8”), characterized by an XRPD pattern comprising peaks 4.0, 7.7, 8.0, 12.3, and 15.1±0.2° 2θ using CuKα radiation.

In some embodiments, the disclosure also provides crystalline forms of Compound A ethanol solvate (“Form 2A”), characterized by an XRPD pattern comprising peaks at 5.8, 11.6, 12.7, 18.0, and 25.7±0.2° 2θ using CuKα radiation.

In some embodiments, the disclosure also provides crystalline forms of Compound A isopropanol solvate (“Form 3A”), characterized by an XRPD pattern comprising peaks at 5.6, 12.8, 16.4, 17.5, and 25.1±0.2° 2θ using CuKα radiation.

In some embodiments, the disclosure also provides crystalline forms of Compound A acetone solvate (“Form 4A”), characterized by an XRPD pattern comprising peaks at 5.7, 7.7, 11.5, 14.8, and 15.3±0.2° 2θ using CuKα radiation.

In some embodiments, the disclosure also provides crystalline forms of Compound A methanol solvate (“Form 5A”), characterized by an XRPD pattern comprising peaks at 4.8, 7.7, 12.3, 15.3, and 16.1±0.2° 2θ using CuKα radiation.

In some embodiments, the disclosure also provides crystalline forms of Compound A methyl tetrahydrofuran solvate (“Form 6A”), characterized by an XRPD pattern comprising peaks at 7.6, 11.3, 15.1, 18.3, and 28.0±0.2° 2θ using CuKα radiation.

In some embodiments, the disclosure also provides an amorphous form Compound A free base, characterized by a differential scanning calorimetry scan substantially as shown in FIG. 105.

In some embodiments, the disclosure also provides crystalline forms of Compound A toluene sulfonate salt (“Form A1”), characterized by an XRPD pattern comprising peaks at 6.0, 19.1, 20.3, 24.1, 24.9, and 28.9±0.2° 2θ using Cu Kα radiation.

In some embodiments, the disclosure also provides crystalline forms of Compound A toluene sulfonate salt (“Form A2”), characterized by an XRPD pattern comprising peaks at 12.7, 15.5, 16.2, 18.7, 19.7, and 21.8±0.2° 2θ using Cu Kα radiation.

In some embodiments, the disclosure also provides crystalline forms of Compound A toluene sulfonate salt (“Form A3”), characterized by an XRPD pattern comprising peaks at 4.9, 15.2, 18.8, 19.5, and 24.5±0.2° 2θ using Cu Kα radiation.

In some embodiments, the disclosure also provides crystalline forms of Compound A benzene sulfonate salt (“Form B1”), characterized by an XRPD pattern comprising peaks at 5.1, 16.1, 17.9, 19.0, and 25.2±0.2° 2θ using Cu Kα radiation.

In some embodiments, the disclosure also provides crystalline forms of Compound A chloride salt (“Form C1”), characterized by an XRPD pattern comprising peaks at 6.8, 11.9, 16.6, 20.7, 23.8, 25.3, and 27.6±0.2° 2θ using Cu Kα radiation.

In some embodiments, the disclosure also provides crystalline forms of Compound A chloride salt (“Form C2”), characterized by an XRPD pattern comprising peaks at 4.2, 5.6, 12.2, 12.9, and 18.1±0.2° 2θ using Cu Kα radiation.

In some embodiments, the disclosure also provides crystalline forms of Compound A sulfate salt (“Form D1”), characterized by an XRPD pattern comprising peaks at 16.0, 16.5, 16.7, 20.0, and 20.4±0.2° 2θ using CuKα radiation.

In some embodiments, the disclosure also provides crystalline forms of Compound A malonate salt (“Form E1”), characterized by an XRPD pattern comprising peaks at 6.8, 12.6, 16.6, 20.4, and 22.0±0.2° 2θ using CuKα radiation.

In some embodiments, the disclosure also provides crystalline forms of Compound A naphthalene-2-sulfonate salt (“Form F1”), characterized by an XRPD pattern comprising peaks at 4.7, 14.7, 14.9, 17.0, 19.6, and 22.1±0.2° 2θ using CuKα radiation.

In some embodiments, the disclosure also provides crystalline forms of Compound A naphthalene-2-sulfonate salt (“Form F2”), characterized by an XRPD pattern comprising peaks at 5.8, 11.6, 14.0, 17.5, and 19.7±0.2° 2θ using CuKα radiation.

In some embodiments, the disclosure also provides crystalline forms of Compound A naphthalene-2-sulfonate salt (“Form F3”), characterized by an XRPD pattern comprising peaks at 3.8, 7.6, 9.7, 11.5, and 15.3±0.2° 2θ using CuKα radiation.

In some embodiments, the disclosure also provides crystalline forms of Compound A methane sulfonate salt (“Form G1”), characterized by an XRPD pattern comprising peaks at 5.1, 6.5, 13.6, 13.8, and 19.5±0.2° 2θ using CuKα radiation.

In some embodiments, the disclosure also provides crystalline forms of Compound A methane sulfonate salt (“Form G2”), characterized by an XRPD pattern comprising peaks at 5.6, 13.1, 13.3, 16.3, and 18.3±0.2° 2θ using CuKα radiation.

In some embodiments, the disclosure also provides crystalline forms of Compound A oxalate salt (“Form H2”), characterized by an XRPD pattern comprising peaks at 6.9, 8.7, 13.6, 17.4, and 24.6±0.2° 2θ using CuKα radiation.

In some embodiments, the disclosure also provides crystalline forms of Compound A tartrate salt (“Form 11”), characterized by an XRPD pattern comprising peaks at 3.4, 14.7, 15.6, 18.02, and 24.3±0.2° 2θ using CuKα radiation.

In some embodiments, the disclosure also provides crystalline forms of Compound A ethanesulfonate salt (“Form J1”), characterized by an XRPD pattern comprising peaks at 5.6, 7.9, 13.6, 15.7, and 17.9±0.2° 2θ using CuKα radiation.

In some embodiments, the disclosure also provides crystalline forms of Compound A ethanesulfonate salt (“Form J2”), characterized by an XRPD pattern comprising peaks at 4.0, 6.3, 7.7, 15.8, and 20.9±0.2° 2θ using CuKα radiation.

In some embodiments, the disclosure also provides crystalline forms of Compound A N-cyclohexylsulfamate salt (“Form K1”), characterized by an XRPD pattern comprising peaks at 5.8, 14.0, 15.6, 16.7, and 28.1±0.2° 2θ using CuKα radiation.

In some embodiments, the disclosure also provides crystalline forms of Compound A maleate salt (“Form L1”), characterized by an XRPD pattern comprising peaks at 5.7, 17.0, 17.5, 25.6, and 26.1±0.2° 2θ using CuKα radiation.

In some embodiments, the disclosure also provides crystalline forms of Compound A phosphate salt (“Form M1”), characterized by an XRPD pattern comprising peaks at 5.4, 16.2, 20.3, and 22.5±0.2° 2θ using CuKα radiation.

In some embodiments, the disclosure also provides crystalline forms of Compound A phosphate salt (“Form M2”), characterized by an XRPD pattern comprising peaks at 7.1, 14.2, 14.9, 17.9, and 19.6±0.2° 2θ using CuKα radiation.

In some embodiments, the disclosure also provides crystalline forms of Compound A phosphate salt (“Form M3”), characterized by an XRPD pattern comprising peaks at 7.5, 7.8, 14.8, 15.0, and 15.4±0.2° 2θ using CuKα radiation.

In some embodiments, the disclosure also provides crystalline forms of Compound A salicylic acid cocrystal (“Form CC-1A”), characterized by an XRPD pattern comprising peaks at 9.3, 5.9, 9.7. 6.0, and 13.9 24±0.2° 2θ using CuKα radiation.

In some embodiments, the disclosure also provides crystalline forms of Compound A salicylic acid cocrystal (“Form CC-2A”), characterized by an XRPD pattern comprising peaks at 8.2, 9.2, 16.5, 18.5, and 16.0±0.2° 2θ using CuKα radiation.

In some embodiments, the disclosure also provides crystalline forms of Compound A salicylic acid cocrystal (“Form CC-3A”), characterized by an XRPD pattern comprising peaks at 3.6, 12.6, 8.0, 14.4, and 7.2±0.2° 2θ using CuKα radiation.

In some embodiments, the disclosure also provides crystalline forms of Compound A salicylic acid cocrystal (“Form CC-4A”), characterized by an XRPD pattern comprising peaks at 10.3, 16.2, 9.9, 16.3, 19.9±0.2° 2θ using CuKα radiation.

In some embodiments, the disclosure also provides crystalline forms of Compound A salicylic acid cocrystal (“Form CC-5A”), characterized by an XRPD pattern comprising peaks at 5.3, 17.8, 10.6, 18.3, and 15.9±0.2° 2θ using CuKα radiation.

In some embodiments, the disclosure also provides crystalline forms of Compound A formic acid cocrystal (“Form CC-1B”), characterized by an XRPD pattern comprising peaks at 8.6, 4.6, 17.8, 17.4, and 23.0±0.2° 2θ using CuKα radiation.

In some embodiments, the disclosure also provides crystalline forms of Compound A benzoic acid cocrystal (“Form CC-1C”), characterized by an XRPD pattern comprising peaks at 11.6, 16.1, 14.2, 3.9, and 19.8±0.2° 2θ using CuKα radiation.

In some embodiments, the disclosure also provides crystalline forms of Compound A isobutyric acid cocrystal (“Form CC-1D”), characterized by an XRPD pattern comprising peaks at 5.6, 6.1, 13.1, 16.0, and 17.1±0.2° 2θ using CuKα radiation.

In some embodiments, the disclosure also provides crystalline forms of Compound A isobutyric acid cocrystal (“Form CC-2D”), characterized by an XRPD pattern comprising peaks at 5.2, 5.5, 6.3, 10.3, and 12.6±0.2° 2θ using CuKα radiation.

In some embodiments, the disclosure also provides crystalline forms of Compound A benzoic acid cocrystal (“Form CC-1C”), characterized by an XRPD pattern comprising peaks at 11.6, 16.1, 14.2, 3.9, and 19.8±0.2° 2θ using CuKα radiation.

In some embodiments, the disclosure also provides crystalline forms of Compound A caprylic acid cocrystal (“Form CC-1E”), characterized by an XRPD pattern comprising peaks at 4.8, 6.2, 6.5, 18.1, and 21.1±0.2° 2θ using CuKα radiation.

In some embodiments, the disclosure also provides crystalline forms of Compound A sorbic acid cocrystal (“Form CC-1F”), characterized by an XRPD pattern comprising peaks at 8.18, 10.8, 11.4, 18.2, and 21.2±0.2° 2θ using CuKα radiation.

In some embodiments, the disclosure also provides crystalline forms of Compound A saccharin cocrystal (“Form CC-1G”), characterized by an XRPD pattern comprising peaks at 5.1, 9.8, 10.1, 16.5, and 20.4±0.2° 2θ using CuKα radiation.

In some embodiments, the disclosure also provides crystalline forms of Compound A succinic acid cocrystal (“Form CC-1H”), characterized by an XRPD pattern comprising peaks at 11.3, 11.5, 18.3, 19.0, and 20.6±0.2° 2θ using CuKα radiation.

In some embodiments, the disclosure also provides crystalline forms of Compound A succinic acid cocrystal (“Form CC-2H”), characterized by an XRPD pattern comprising peaks at 4.2, 5.3, 8.3, 9.0, and 9.2±0.2° 2θ using CuKα radiation.

In some embodiments, the disclosure also provides crystalline forms of Compound A adipic acid cocrystal (“Form CC-1I”), characterized by an XRPD pattern comprising peaks at 7.7, 10.5, 18.5, 18.9, and 21.7±0.2° 2θ using CuKα radiation.

In some embodiments, the disclosure also provides pharmaceutical compositions comprising the crystalline forms, amorphous forms, salts, cocrystals, and solvates of Compound A disclosed herein and at least one pharmaceutically acceptable excipient.

In some embodiments, the disclosure also provides methods of treating cancer in a subject comprising administering to the subject a therapeutically effective amount of the crystalline forms amorphous forms, salts, cocrystals, and solvates of Compound A disclosed herein.

### BRIEF DESCRIPTION OF THE DRAWINGS

FIG. 1 depicts an XRPD pattern of a crystalline form of Compound A free base Form 1.

FIG. 2 depicts a differential scanning calorimetry (DSC) thermograph and a thermogravimetric analysis (“TGA”) trace of a crystalline form of Compound A free base Form 1 indicating an extrapolated onset at 225° C. and a weight loss of 0.03% from 35-150° C.

FIG. 3 depicts a moisture sorption profile (DVS) of a crystalline form of Compound A free base Form 1 showing non-hygroscopicity between 0% to 95% relative humidity at 25° C.

FIG. 4 depicts an XRPD pattern of a crystalline form of Compound A free base Form 3.

FIG. 5 depicts a DSC thermograph and a thermogravimetric analysis (“TGA”) trace of a crystalline form of Compound A free base Form 3 indicating exothermic events with onset temperatures of 105° C. and 173° C. and peak temperatures of 111° C. and 173° C.; an endothermic event is observed with onset and peak temperatures of 226° C. and 229° C. Negligible weight loss is shown up to 200° C.

FIG. 6 depicts a DVS of a crystalline form of Compound A free base Form 3.

FIG. 7 depicts an XRPD pattern of a crystalline form of Compound A free base Form 6.

FIG. 8 depicts a DSC thermograph and a thermogravimetric analysis (“TGA”) trace of a crystalline form of Compound A free base Form 6.

FIG. 9 depicts an XRPD pattern of a crystalline form of Compound A free base Form 7.

FIG. 10 depicts a DSC thermograph and a thermogravimetric analysis (“TGA”) trace of a crystalline form of Compound A free base Form 7 indicating endothermic events with onset temperatures of 61° C. and others at higher temperatures with peak temperatures at 87° C., 221° C., and 223° C. An exothermic event was observed with onset and peak temperatures of 129° C. and 141° C. A weight loss of 1.2% was observed at 94° C. and an additional weight loss of 0.3% up to 176° C.

FIG. 11 depicts an XRPD pattern of a crystalline form of Compound A free base Form 8 hydrate.

FIG. 12 depicts a DSC thermograph and a thermogravimetric analysis (“TGA”) trace of a crystalline form of Compound A free base Form 8 hydrate indicating endothermic events with onset temperatures of 57° C. and 219° C. and peak temperatures of 79° C. and 225° C., respectively. A weight loss of 3.3% was observed by TGA up to 128° C.

FIG. 13 depicts an XRPD pattern of a crystalline form of Compound A ethanol solvate Form 2A.

FIG. 14 depicts a DSC thermograph and a thermogravimetric analysis (“TGA”) of a crystalline form of Compound A ethanol solvate Form 2A. DSC shows the first endotherm with an extrapolated onset at 132° C. as a result of desolvation of ethanol, and the second endotherm overlapped with the exotherm with subsequent recrystallization and was followed by an extrapolated melting onset at 224° C. and weight loss of 2.9% from 26-160° C.

FIG. 15 depicts an XRPD pattern of a crystalline form of Compound A isopropanol solvate Form 3A.

FIG. 16 depicts a DSC thermograph of a crystalline form of Compound A isopropanol solvate Form 3A and a thermogravimetric analysis (TGA). DSC shows the first endotherm with an extrapolated onset at 123° C. as a result of desolvation of isopropyl alcohol (IPA), and the second endotherm overlapped with the exotherm with subsequent recrystallization and followed by an extrapolated melting onset at 223° C. and a weight loss of 6.6% from 28-150° C.

FIG. 17 depicts an XRPD pattern of a crystalline form of Compound A acetone solvate Form 4A.

FIG. 18 depicts a DSC thermograph of a crystalline form of Compound A acetone solvate Form 4A and a thermogravimetric analysis (TGA). DSC shows endothermic events with onset temperatures of 111° C., 143° C., and 226° C. and peak temperatures of 124° C., 151° C., and 227° C. An exothermic event is observed with onset and peak temperatures of 158° C. and 162° C. A weight loss of 4.2% was observed by TGA up to 152° C.

FIG. 19 depicts an XRPD pattern of a crystalline form of Compound A methanol solvate Form 5A.

FIG. 20 depicts a DSC thermograph of a crystalline form of Compound A methanol solvate Form 5A and a thermogravimetric analysis (TGA). DSC shows endothermic events with onset temperatures of 50° C. and 226° C. and peak temperatures of 70° and 227° C. An exothermic event was observed with onset and peak temperatures of 117° C. and 137° C. A weight loss of 1.7% was observed by TGA up to 200° C.

FIG. 21 depicts an XRPD pattern of a crystalline form of Compound A toluene sulfonate salt Form A1.

FIG. 22 depicts a DSC thermograph and a thermogravimetric analysis (TGA) of a crystalline form of Compound A toluene sulfonate salt Form A1 indicating an extrapolated onset of 293° C. and weight loss of 0.15% from 32-200° C.

FIG. 23 depicts a DVS of a crystalline form of Compound A toluene sulfonate Form A1 showing a weight gain of 0.5% by 90% relative humidity at 25° C.

FIG. 24 depicts an XRPD pattern of a crystalline form of Compound A toluene sulfonate salt Form A2.

FIG. 25 depicts a DSC thermograph and a thermogravimetric analysis (“TGA”) of a crystalline form of Compound A toluene sulfonate salt Form A2. DSC shows an exotherm with an extrapolated onset at 242° C. due to recrystallization and followed by an extrapolated melt/decomposition at 287° C. and weight loss of 0.04% from 32-150° C.

FIG. 26 depicts an XRPD pattern of crystalline form of Compound A toluene sulfonate salt Form A3.

FIG. 27 depicts a DSC thermograph and a thermogravimetric analysis (“TGA”) of crystalline form of Compound A toluene sulfonate salt Form A3. DSC shows an endothermic event with onset and peak temperatures of 281° C. and 289° C., respectively. Form A3 exhibited a negligible weight loss by TGA up to 256.4° C.

FIG. 28 depicts a DVS of a crystalline form of Compound A toluene sulfonate salt Form A3

FIG. 29 depicts an XRPD pattern of a crystalline form of Compound A benzene sulfonate salt Form B1.

FIG. 30 depicts a DSC thermograph of a crystalline form of Compound A benzene sulfonate salt Form B1. DSC shows an endotherm with an extrapolated onset at 97° C. followed by an extrapolated endothermic melt/decomposition at 254° C.

FIG. 31 depicts an XRPD pattern of a crystalline form of Compound A hydrochloride salt Form C1.

FIG. 32 depicts a DSC thermograph and a thermogravimetric analysis (TGA) of a crystalline form of Compound A chloride salt Form C1. DSC shows a broad endotherm with an extrapolated onset at 34° C. and an extrapolated endothermic melt/decomposition/salt disproportionation at 166° cand weight loss of 12.6% from 30-200° C.

FIG. 33 depicts an XRPD pattern of crystalline form of Compound A chloride salt Form C2.

FIG. 34 depicts a DSC thermograph and a thermogravimetric analysis (TGA) of crystalline Compound A chloride salt Form C2. DSC shows endothermic melts with onset temperatures of 54° C. and 121° C. and peak temperatures of 75° C. and 133° C., respectively. Form C2 exhibited a weight loss of 8.6% observed up to 212° C. by TGA.

FIG. 35 depicts an XRPD pattern of a crystalline form of Compound A sulfate salt Form D1.

FIG. 36 depicts an XRPD pattern of crystalline Compound A malonate salt Form E1.

FIG. 37 depicts a DSC thermograph and a thermogravimetric analysis (TGA) of crystalline Compound A malonate salt Form E1. DSC shows endothermic melt with onset and peak temperatures of 182° C. and 183° C., respectively. Form E1 shows a negligible weight loss by TGA up to 153° C.

FIG. 38 depicts an XRPD pattern of crystalline Compound A naphthalene-2-sulfonate salt Form F1.

FIG. 39 depicts a DSC thermograph and a thermogravimetric analysis (TGA) of crystalline Compound A naphthalene-2-sulfonate salt Form F1. DSC shows endothermic melt with onset and peak temperatures of 206° C. and 211° C., respectively. Form F1 shows negligible weight loss by TGA up to 212° C.

FIG. 40 depicts a DVS of crystalline Compound A naphthalene-2-sulfonate salt Form F1.

FIG. 41 depicts an XRPD pattern of crystalline Compound A naphthalene-2-sulfonate salt Form F2.

FIG. 42 depicts a DSC thermograph and a thermogravimetric analysis (TGA) of crystalline Compound A naphthalene-2-sulfonate salt Form F2. DSC shows endothermic melt with onset and peak temperatures of 273° C. and 280° C. Form F2 shows negligible weight loss up to 223° C. by TGA.

FIG. 43 depicts a DVS of crystalline Compound A naphthalene-2-sulfonate salt Form F2.

FIG. 44 depicts an XRPD pattern of crystalline Compound A naphthalene-2-sulfonate salt Form F3.

FIG. 45 depicts a DSC thermograph and a thermogravimetric analysis (TGA) of crystalline Compound A naphthalene-2-sulfonate salt Form F3. DSC shows endothermic events with onset temperatures of 50° C. and 147° C. and peak temperatures of 76° C. and 212° C., respectively. Form F3 shows a weight loss of 3.6% by TGA up to 209° C.

FIG. 46 depicts an XRPD pattern of crystalline Compound A methane sulfonate salt Form G1.

FIG. 47 depicts an XRPD pattern of crystalline Compound A methane sulfonate salt Form G2.

FIG. 48 depicts a DSC thermograph and a thermogravimetric analysis (TGA) of crystalline Compound A methane sulfonate salt Form G2. DSC shows an endothermic transition with onset and peak temperatures of 271° C. and 272° C., respectively. Form G3 shows negligible weight loss by TGA up to 206° C.

FIG. 49 depicts a DVS of crystalline Compound A methane sulfonate salt Form G2.

FIG. 50 depicts an XRPD pattern of crystalline Compound A oxalate salt Form H2.

FIG. 51 depicts a DSC thermograph and a thermogravimetric analysis (TGA) of crystalline Compound A oxalate salt Form H2. DSC shows an endothermic event with onset and peak temperatures of 241° C. and 243° C., respectively. Form H2 shows negligible weight loss by TGA up to 196° C.

FIG. 52 depicts an XRPD pattern of crystalline Compound A tartrate salt Form I1.

FIG. 53 depicts a DSC thermograph and a thermogravimetric analysis (TGA) of crystalline Compound A tartrate salt Form I1. DSC shows endothermic events with onset temperatures of 79° C. and 144° C. and peak temperatures of 105° C. and 152° C., respectively. Form I1 shows a weight loss of 3.0% by TGA up to 151° C.

FIG. 54 depicts an XRPD pattern of crystalline Compound A ethanesulfonate salt Form J1.

FIG. 55 depicts a DSC thermograph and a TGA of crystalline Compound A ethanesulfonate salt Form J1. DSC shows endothermic events with onset temperatures of 30° C. and 244° C. and peak temperatures of 64° C. and 254° C. Form J1 shows a weight loss of 6.2% by TGA up to 250° C.

FIG. 56 depicts an XRPD pattern of crystalline Compound A ethanesulfonate salt Form J2.

FIG. 57 depicts a DSC thermograph and a TGA of crystalline Compound A ethanesulfonate salt Form J2. DSC shows endothermic events with onset temperatures of 34.4° C. and 238.0° C. and peak temperatures of 63° C. and 249° C., respectively. Form J2 shows a weight loss of 2.5% by TGA up to 219° C.

FIG. 58 depicts an XRPD pattern of crystalline Compound A N-cyclohexylsulfamate salt Form K1.

FIG. 59 depicts a DSC thermograph and a TGA of crystalline Compound A N-cyclohexylsulfamate salt Form K1. DSC shows endothermic event with onset and peak temperatures of 201° C. and 211° C., respectively. Form K1 shows a weight loss of 2.5% by TGA up to 213° C.

FIG. 60 depicts an XRPD pattern of crystalline Compound A maleate salt Form L1.

FIG. 61 depicts a DSC thermograph and a TGA of crystalline Compound A maleate salt Form L1. DSC shows endothermic event with onset and peak temperatures of 198° C. and 203° C. Form L1 shows a weight loss of 2.0% by TGA up to 181° C.

FIG. 62 depicts an XRPD pattern of crystalline Compound A phosphate salt Form M1.

FIG. 63 depicts a DSC thermograph and a TGA of crystalline Compound A phosphate salt Form M1. DSC shows an endothermic event with an onset temperature 214° C. and peak temperature of 217° C. Form M1 shows negligible weight loss by TGA up to 188° C.

FIG. 64 depicts a DVS of crystalline Compound A phosphate salt Form M1.

FIG. 65 depicts an XRPD pattern of crystalline Compound A phosphate salt Form M2.

FIG. 66 depicts a DSC thermograph and a TGA of crystalline Compound A phosphate salt Form M2. DSC shows endothermic events with onset temperatures of 57° C., 88° C., 141° C., and 198° C. and peak temperatures of 74° C., 109° C., 149° C., and 205° C. Form M2 shows a weight loss of 1.1% by TGA up to 106° C.

FIG. 67 depicts an XRPD pattern of crystalline Compound A phosphate salt Form M3.

FIG. 68 depicts a DSC thermograph and a TGA of crystalline Compound A phosphate salt Form M3. DSC shows endothermic events with peak temperatures of 69° C., 88° C., and 102° C. In addition, there are endothermic events with onset temperatures of 157° C. and 208° C. and peak temperatures of 165° C. and 214° C., respectively. There is an exothermic event with onset and peak temperatures of 174° C. and 181° C., respectively. Form M3 shows a weight loss of 8.1% weight loss by TGA.

FIG. 69 depicts an XRPD pattern of crystalline Compound A salicylic acid cocrystal Form CC-1A.

FIG. 70 depicts a DSC thermograph and a TGA of crystalline Compound A salicylic acid cocrystal Form CC-1A. DSC shows endothermic events with onset temperatures of 43° C. and 103° C. and peak temperatures of 75° C., 107° C. and 239° C., respectively. Form CC1-A shows a weight loss of 11.7% by TGA up to 126° C.

FIG. 71 depicts an XRPD pattern of crystalline Compound A salicylic acid cocrystal Form CC-2A.

FIG. 72 depicts a DSC thermograph and a TGA of crystalline Compound A salicylic acid cocrystal Form CC-2A. DSC shows endothermic events with onset temperatures of 107° C. and 195° C. and peak temperatures of 119° C. and 196° C., respectively. Form CC2-A shows a weight loss of 13.6% by TGA up to 133° C.

FIG. 73 depicts an XRPD pattern of crystalline Compound A salicylic acid cocrystal Form CC-3A.

FIG. 74 depicts a DSC thermograph and a TGA of crystalline Compound A salicylic acid cocrystal Form CC-3A. DSC shows an endothermic event with an onset and peak temperature of 182° C. and 185° C., respectively. Form CC-3A shows negligible weight loss by TGA up to 127° C.

FIG. 75 depicts a DVS of crystalline Compound A salicylic acid cocrystal Form CC-3A, which has a 1.3% mass increase up to 80% relative humidity.

FIG. 76 depicts an XRPD pattern of crystalline Compound A salicylic acid cocrystal Form CC-4A.

FIG. 77 depicts a DSC thermograph and a TGA of crystalline Compound A salicylic acid cocrystal Form CC-4A. DSC shows an endothermic event with an onset and peak temperature of 143° C. and 198° C. and peak temperatures of 152° C. and 200° C., respectively. Form CC-4A shows a weight loss of 5.7% by TGA up to 171° C.

FIG. 78 depicts an XRPD pattern of crystalline Compound A salicylic acid cocrystal Form CC-5A.

FIG. 79 depicts a DSC thermograph and a TGA of crystalline Compound A salicylic acid cocrystal Form CC-5A. DSC shows an endothermic event with a peak temperature of 1224° C. Form CC-5A shows a weight loss of 8.3% by TGA up to 134° C.

FIG. 80 depicts an XRPD pattern of crystalline Compound A formic acid cocrystal Form CC-1B.

FIG. 81 depicts a DSC thermograph and a TGA of crystalline Compound A formic acid cocrystal Form CC-1B. DSC shows endothermic events with peak temperatures of 62° C. and 105° C. Form CC-1B shows a weight loss of 1.1% up to 63° C. is observed by TGA.

FIG. 82 depicts an XRPD pattern of crystalline Compound A benzoic acid cocrystal Form CC-1C.

FIG. 83 depicts a DSC thermograph and a TGA of crystalline Compound A benzoic acid cocrystal Form CC-1C. DSC shows endothermic melt with onset and peak temperatures of 186° C. and 187° C., respectively. Form CC-1C shows negligible weight loss up to 117° C. by TGA.

FIG. 84 depicts a DVS of crystalline Compound A benzoic acid cocrystal Form CC-1C, which has a 0.3% mass increase up to 80% relative humidity.

FIG. 85 depicts an XRPD pattern of crystalline Compound A isobutyric acid cocrystal Form CC-1D.

FIG. 86 depicts a DSC thermograph and a TGA of crystalline Compound A isobutyric acid cocrystal Form CC-1D. DSC shows endothermic events with onset temperatures of 155° C. and 226° C. and peak temperatures of 176° C. and 227° C., respectively. Form CC-1D shows a weight loss of 16.3% by TGA up to 180° C.

FIG. 87 depicts a DVS of crystalline Compound A isobutyric acid cocrystal Form CC-1D, which has a 0.2% mass increase up to 80% relative humidity.

FIG. 88 depicts an XRPD pattern of crystalline Compound A isobutyric acid cocrystal Form CC-2D.

FIG. 89 depicts a DSC thermograph and a TGA of crystalline Compound A isobutyric acid cocrystal Form CC-2D. DSC shows endothermic events with onset temperatures of 163° C. and 228° C. Form CC-2D shows a weight loss of 18.1% by TGA up to 192° C.

FIG. 90 depicts a DVS of crystalline Compound A isobutyric acid cocrystal Form CC-2D, which has a 0.2% mass increase up to 80% relative humidity.

FIG. 91 depicts an XRPD pattern of crystalline Compound A caprylic acid cocrystal Form CC-1E.

FIG. 92 depicts a DSC thermograph and a TGA of crystalline Compound A caprylic acid cocrystal Form CC-1E. DSC shows an endothermic event with onset and peak temperatures of 131° C. and 136° C., respectively. Concurrent degradation is observed by TGA with the endothermic event in the DSC.

FIG. 93 depicts an XRPD pattern of crystalline Compound A sorbic acid acid cocrystal Form CC-1F.

FIG. 94 depicts a DSC thermograph and a TGA of crystalline Compound A sorbic acid acid cocrystal Form CC-1F. DSC shows endothermic events with onset temperatures of 62° C. and 160° C. and peak temperatures of 90° C. and 170° C., respectively. Form CC-1F shows a weight loss of 20.8% by TGA up to 218° C.

FIG. 95 depicts XRPD pattern of crystalline Compound A saccharin cocrystal Form CC-1G.

FIG. 96 depicts a DSC thermograph and a TGA of crystalline Compound A saccharin cocrystal Form CC-1G. DSC shows an endothermic event with onset and peak temperatures of 243° C. and 244° C., respectively. Form CC-1G shows negligible weight loss by TGA up to 182° C.

FIG. 97 depicts a DVS of crystalline Compound A saccharin cocrystal Form CC-1G, which has a 0.2% mass increase up to 80% relative humidity.

FIG. 98 depicts an XRPD pattern of crystalline Compound A succinic acid cocrystal Form CC-1H.

FIG. 99 depicts a DSC thermograph and a TGA of crystalline Compound A succinic acid cocrystal Form CC-1H. DSC shows endothermic melts with onset temperatures of 59° C. and 186° C. and peak temperatures of 82° C. and 187° C., respectively. Form CC-1H shows a weight loss of 2.9% by TGA up to 102° C.

FIG. 100 depicts an XRPD pattern of crystalline Compound A succinic acid cocrystal Form CC-2H.

FIG. 101 depicts a DSC thermograph and a TGA of crystalline Compound A succinic acid cocrystal Form CC-2H. DSC shows endothermic transitions with onset temperatures of 94° C. and 166° C. and peak temperatures of 99° C. and 172° C., respectively. Form CC-2H shows a weight loss of 24.4% by TGA up to 235° C.

FIG. 102 depicts an XRPD pattern of crystalline Compound A adipic acid cocrystal Form CC-1I.

FIG. 103 depicts a DSC thermograph and a TGA of crystalline Compound A adipic acid cocrystal Form CC-1I. DSC shows endothermic melts with onset temperatures of 91° C. and 137° C. and peak temperatures of 91° C. and 140° C., respectively. Form CC-1I shows a weight loss of 2.7% by TGA up to 78° C.

FIG. 104 depicts an XRPD pattern of amorphous form Compound A free base.

FIG. 105 depicts a modulated differential scanning calorimetry (mDSC) thermograph of amorphous form Compound A free base indicating a Tg of 119° C.

### DETAILED DESCRIPTION

Disclosed herein are solid forms of Compound A having the following structure:

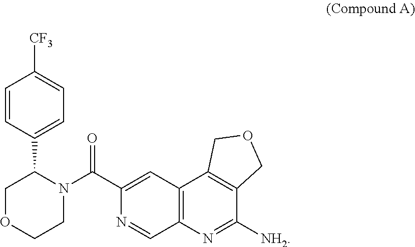 [SMILES: Nc1nc2cnc(C(=O)N3CCOC[C@@H]3c3ccc(C(F)(F)F)cc3)cc2c2c1COC2]

Compound A is a small molecule MTA cooperative PRMT5 inhibitor in development for the treatment of MTAP-null cancers with high unmet need such as, for example, squamous cell non-small cell lung cancer (NSCLC) and pancreatic cancer. The disclosed solid forms include crystalline forms and amorphous forms of Compound A free base, as well as crystalline salts, cocrystals, and solvates of Compound A. The disclosed solid forms can have unique physical properties which are advantageous for new pharmaceutical compositions of Compound A.

Also disclosed herein are pharmaceutical compositions comprising the disclosed solid forms of Compound A and methods of treating a subject suffering from cancer, comprising administering to the subject a therapeutically effective amount of the disclosed solid forms of Compound A.

International Patent Application Nos. PCT/US22/75648 and PCT/US21/63540, each of which is incorporated by reference herein in its entirety, discloses synthetic procedures for synthesizing PRMT5 inhibitors, such as Compound A.

Applicant has discovered the various solid forms disclosed herein using high throughput (HT) screenings, including a HT salt screen and a HT polymorph screen. For example, various crystalline salts and free base polymorphs were identified, including, for example, benzenesulfonic acid (BSA) salt, and methanesulfonic acid (MSA) salt, toluenesulfonic acid (TSA) salt, maleate salt, ethanesulfonic acid (ESA) salt, ethanedisulfonic acid (EDSA) salt, hydrochloride (HCl) salt, sulfate (H₂SO₄) salt and bromide salt (HBr). In addition, new XRPD groups related to free base were also identified from the HT salt screening.

As used herein, the term “solid forms” refers to crystalline forms, amorphous forms, salts, cocrystals, or solvates, including but not limited to the specific solid forms disclosed herein. In some instances, the term “crystalline form” is used herein to refer to the various crystalline forms disclosed herein (e.g., free base forms, salts, solvates, and/or cocrystals).

As used herein, the term “salt” refers to a zwitterionic compound consisting of a cation and an anion. As used herein, the term “pharmaceutically acceptable salt refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of the disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other illustrative pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N+ (C1-4alkyl)4 salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “co-crystal” refers to a crystalline material comprising two or more compounds at ambient temperature (e.g, 20-25° C.), of which at least two are held together by weak interaction, wherein at least one of the compounds is a co-crystal former and the other is Compound 1. Weak interaction is being defined as an interaction which is neither ionic nor covalent and includes for example: hydrogen bonds, van der Waals forces, and pi-pi interactions. The term “co-crystal” includes solvate forms.

As used herein, the term “amorphous form” or “amorphous” means a material that lacks long range order and as such does not show distinct X-ray diffraction peaks, i.e., a Bragg diffraction peak. The XRPD pattern of an amorphous material is characterized by one or more amorphous halos. As used herein, the term “amorphous halo” refer to an approximately bell-shaped maximum in the X-ray powder pattern of an amorphous substance.

As used herein to refer to DSC data, “substantially” refers to a variance of +3° C.

As used herein, the terms “pharmaceutical composition” and “pharmaceutical formulation” are used interchangeably.

The disclosed solid forms of Compound A were prepared and analyzed as described in the Examples using one or more of the following techniques: X-ray powder diffraction (XRPD), differential scanning calorimetry (DSC), thermogravimetric analysis (TGA), dynamic vapor sorption (DVS), nuclear magnetic resonance spectroscopy (NMR) (e.g., ¹³C NMR).

### Free Base Forms

### Crystalline Compound A Free Base (“Form 1”)

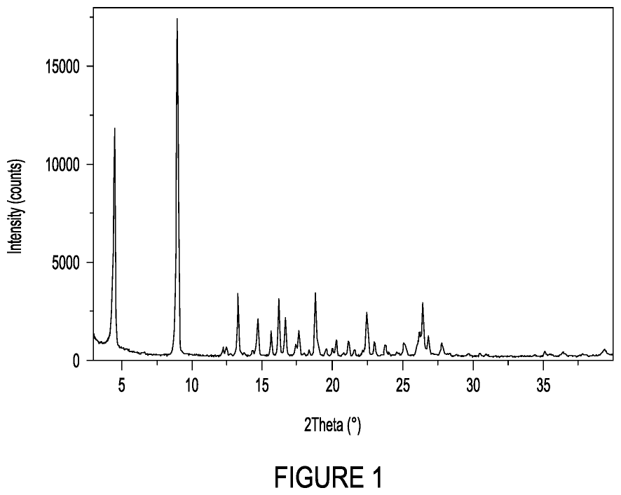

In some embodiments, the disclosure provides Compound A as the free base, wherein the free base is crystalline (e.g., crystalline Compound A free base). In some embodiments, the disclosure provides a crystalline form of Compound A free base (“Form 1”) characterized by an XRPD pattern comprising peaks at 4.5, 9.0, 13.3, 16.2, and 18.9±0.2° 2θ using CuKα radiation. In some embodiments, Form 1 is further characterized by XRPD pattern peaks at 14.7, 15.7, 16.7, 17.6, 22.5, 26.2, and 26.4±0.2° 2θ using CuKα radiation. In some embodiments, Form 1 has an XRPD pattern substantially as shown in FIG. 1.

In some embodiments, Form 1 is characterized by an XRPD pattern comprising at least three peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

In some embodiments, Form 1 is characterized by an XRPD pattern comprising at least five peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

In some embodiments, Form 1 is characterized by an XRPD pattern comprising at least seven peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

In some embodiments, Form 1 is characterized by an XRPD pattern comprising at least eight peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

In some embodiments, Form 1 is characterized by ¹³C solid state NMR comprising at least three peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

In some embodiments, Form 1 is characterized by ¹³C solid state NMR comprising at least five peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

In some embodiments, Form 1 is characterized by ¹³C solid state NMR comprising at least seven peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

In some embodiments, Form 1 is characterized by ¹⁹F solid state NMR comprising peaks at −62.0 and −63.9 ppm.

Single crystals of Compound A free base Form 1 were grown by slow evaporation from ethyl acetate and used for single crystal X-ray structure determination. The results confirmed it is an anhydrous form crystallizing in a monoclinic chiral C2 space group with two Compound A molecules in the asymmetric unit (Z′=2). The absolute configuration was confirmed, and the chiral carbon is in the S-configuration for all molecules in the unit cell (Z=8).

In some embodiments, Form 1 has unit cell dimensions of a=40.410 (2) Å; b=7.0626 (3) Å; c=14.5109 (6) Å; α=90°; β=106.722 (3)°; and γ=90°.

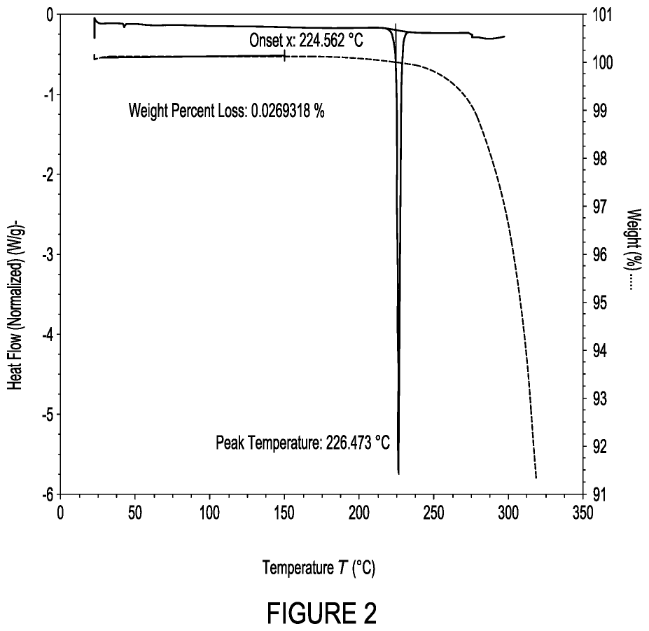

Alternatively, or in addition to, Form 1 can be characterized using differential scanning calorimetry (DSC). In some embodiments, Form 1 has a DSC substantially as shown in FIG. 2. Differential scanning calorimetry (DSC) thermographs were obtained for Form 1, as set forth in the Examples. The DSC curve indicates an endothermic transition at 218° C. to 235° C. In some embodiments, Form 1 has an extrapolated onset at 225° C.±3° C. Form 1 shows a neglible weight loss by TGA up to 226° C.

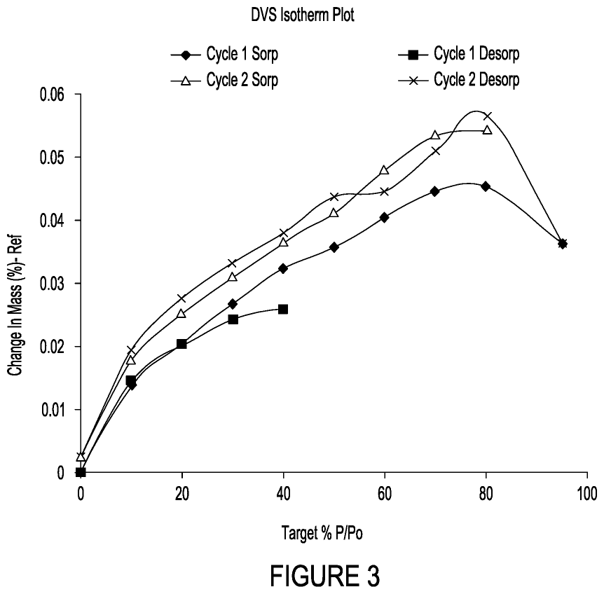

Alternatively, or in addition to, the crystalline Compound A free base Form 1 is characterized using thermogravimetric analysis (TGA). In some embodiments, crystalline Compound A free base Form 1 has a Dynamic Vapor Sorption (DVS) substantially as shown in FIG. 3.

In addition, Form 1 is stable under compression forces representative of tableting processes. By way of example, Form 1 exhibits no form change by DSC and XRPD after compression up to 200 MPa. Moreover, Form 1 demonstrates suitable stability. By way of example, Form 1 is physically and chemically stable, as determined by ssNMR, in studies of various excipient blends under various conditions including, for example, 40° C./75% RH, 25° C./60% RH open, and 40° C./75% RH closed conditions, wherein no evidence of degradation related to the Maillard reaction was identified after 2 weeks. Furthermore, Form 1 has a high solubility in many organic solvents, especially with water as a co-solvent (e.g., acetonitrile (13.2 mg/ml), 1:1 acetonitrile/water (31.1 mg/mL), ethanol 4.8 mg/mL), 1:1 ethanol/water (15.9 mg/mL), acetone (33.0 mg/mL), 1:4 acetone/water (0.2 mg/mL), and isopropanol (9.1 mg/mL)). Solubility studies demonstrate that Form 1 exhibits a desirable solubility of greater than 10 μg/mL in aqueous media at pHs of 1-8.

### Crystalline Compound A Free Base (“Form 3”)

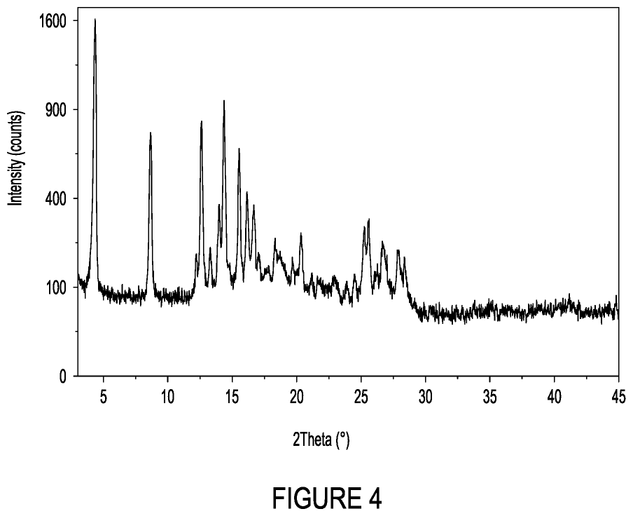

In some embodiments, the disclosure provides a crystalline form of Compound A free base (“Form 3”) characterized by an XRPD pattern comprising peaks at 4.3, 12.6, 14.4, 16.2, and 25.6±0.2° 2θ using CuKα radiation. In some embodiments, Form 3 is further characterized by XRPD pattern peaks at 8.6, 13.9, 15.6, 16.7, and 25.3±0.2° 2θ using CuKα radiation. In still other embodiments, Form 3 is further characterized by XRPD pattern peaks at 18.4, 19.7, 20.3, 26.7, 27.9, and 28.4±0.2° 2θ using CuKα radiation. In some embodiments, Form 3 has an XRPD pattern substantially as shown in FIG. 4.

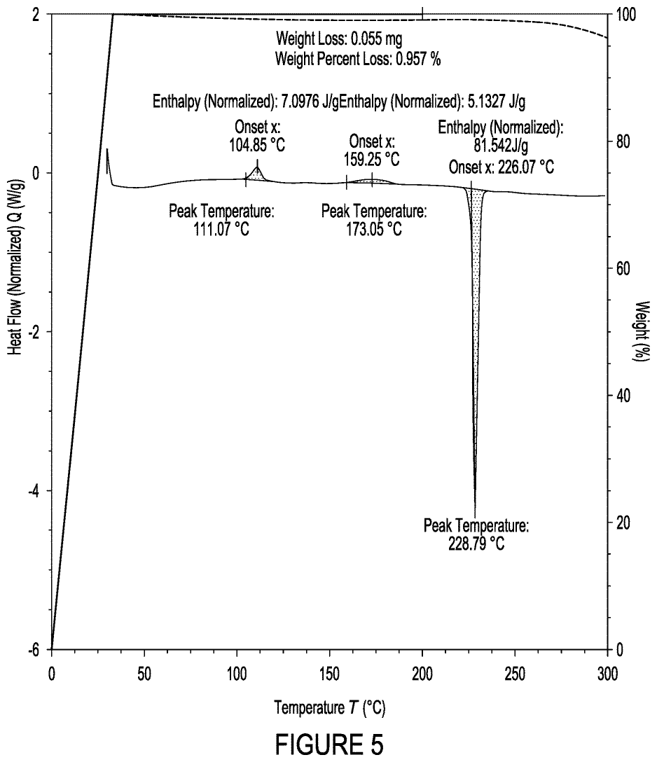

Alternatively, or in addition to, Form 3 can be characterized using DSC. In some embodiments, Form 3 has a DSC substantially as shown in FIG. 5. The DSC thermographs were obtained for Form 3, as set forth in the Examples. The DSC curve indicates exothermic events with onset temperatures of 105° C. and 173° C. and peak temperatures of 111° C. and 173° C. An endothermic event was observed with onset and peak temperatures of 226° C. and 229° C.

Alternatively, or in addition to, Form 3 is characterized using thermogravimetric analysis (TGA). In some embodiments, Compound A free base Form 3 has a TGA substantially as shown in FIG. 5. As shown in FIG. 5, Form 3 exhibited negligible weight loss up to 200° C.

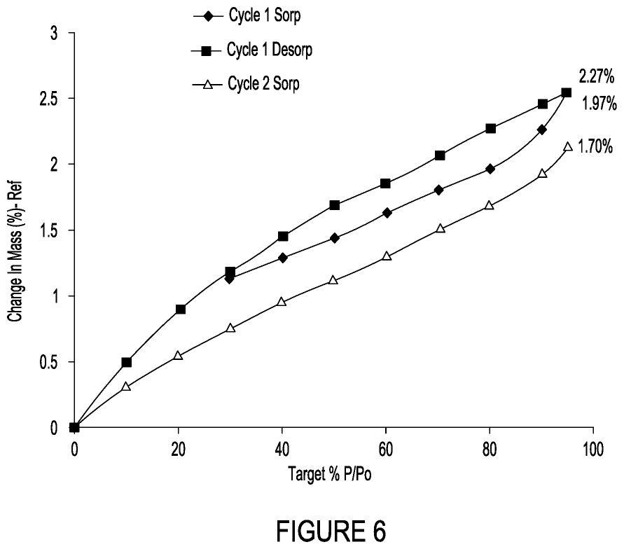

Alternatively, or in addition to, Form 3 is characterized using DVS. In some embodiments, Form 3 has a DVS isotherm plot as shown in FIG. 6.

### Crystalline Compound A Free Base (“Form 6”)

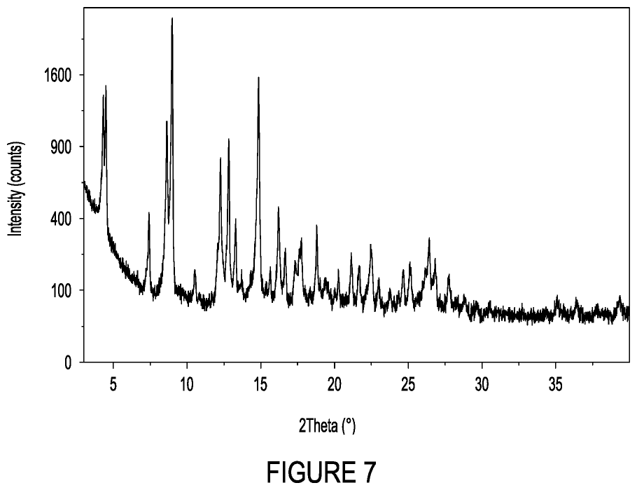

In some embodiments, the disclosure provides a crystalline form of Compound A free base (“Form 6”) characterized by an XRPD pattern comprising peaks at 4.5, 8.6, 9.0, 12.9, and 14.9±0.2° 2θ using CuKα radiation. In some embodiments, Form 6 is further characterized by XRPD pattern peaks at 7.4, 12.3, 13.3, 16.2, 17.8, and 18.8±0.2° 2θ using CuKα radiation. In some embodiments, Form 6 has an XRPD pattern substantially as shown in FIG. 7.

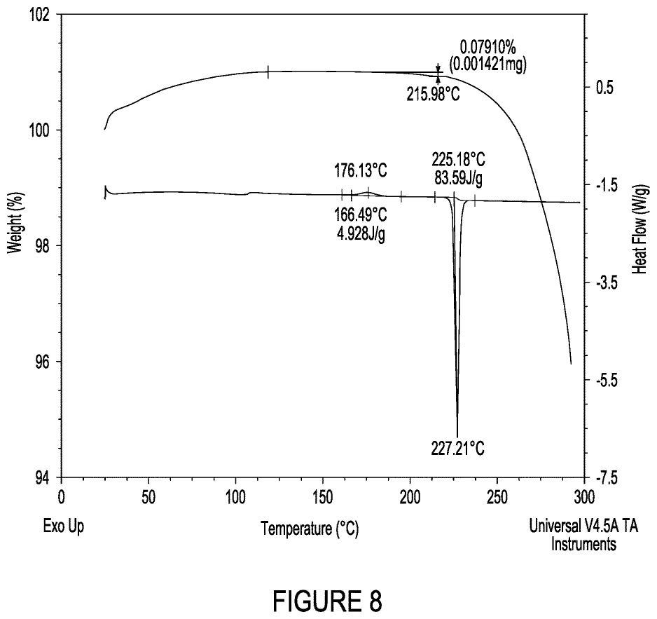

Alternatively, or in addition to, Form 6 can be characterized using DSC. In some embodiments, Form 6 has a DSC substantially as shown in FIG. 8. Differential scanning calorimetry (DSC) thermographs were obtained for Form 6, as set forth in the Examples. An endothermic event was observed with onset and peak temperatures of 225° C. and 227° C., as shown in FIG. 8.

Alternatively, or in addition to, Form 6 is characterized using thermogravimetric analysis (TGA). In some embodiments, Compound A free base Form 6 has a TGA substantially as shown in FIG. 8. As shown in FIG. 8, Form 6 exhibited negligible weight loss up to 216° C.

### Crystalline Compound A Free Base (“Form 7”)

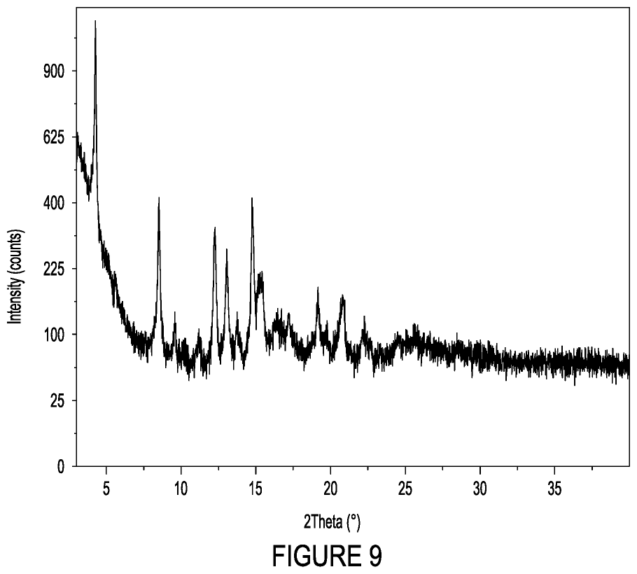

In some embodiments, the disclosure provides a crystalline form of Compound A free base (“Form 7”) characterized by an XRPD pattern comprising peaks at 4.3, 8.5, 12.3, 13.1, and 14.8±0.2° 2θ using CuKα radiation. In some embodiments, Form 7 is further characterized by XRPD pattern peaks at 15.5, 19.1, and 20.9±0.2° 2θ using CuKα radiation. In some embodiments, Form 7 has an XRPD pattern substantially as shown in FIG. 9.

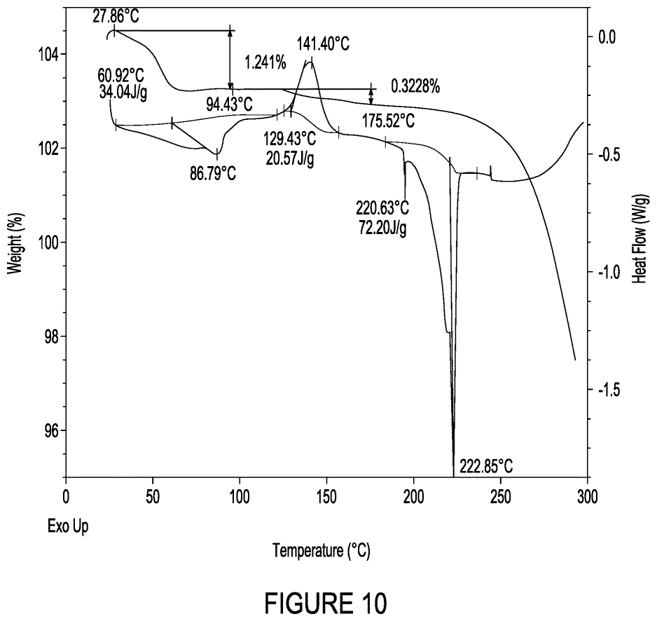

Alternatively, or in addition to, Form 7 can be characterized using DSC. In some embodiments, Form 7 has a DSC substantially as shown in FIG. 10. Differential scanning calorimetry (DSC) thermographs were obtained for Form 7, as set forth in the Examples.

Alternatively, or in addition to, Form 7 is characterized using thermogravimetric analysis (TGA). In some embodiments, Compound A free base Form 7 has a TGA substantially as shown in FIG. 10.

As shown in FIG. 10, Form 7 exhibited endothermic events with onset temperatures of 61° C. and others at higher temperatures. Endothermic events had peak temperatures at 87° C., 221° C., and 223° C. An exothermic event was observed with onset and peak temperatures of 129° C. and 141° C. Moreover, Form 7 exhibited a weight loss of 1.2% by TGA up to 94° C. and an additional weight loss of 0.3% up to 176° C.

### Amorphous Compound A Free Base

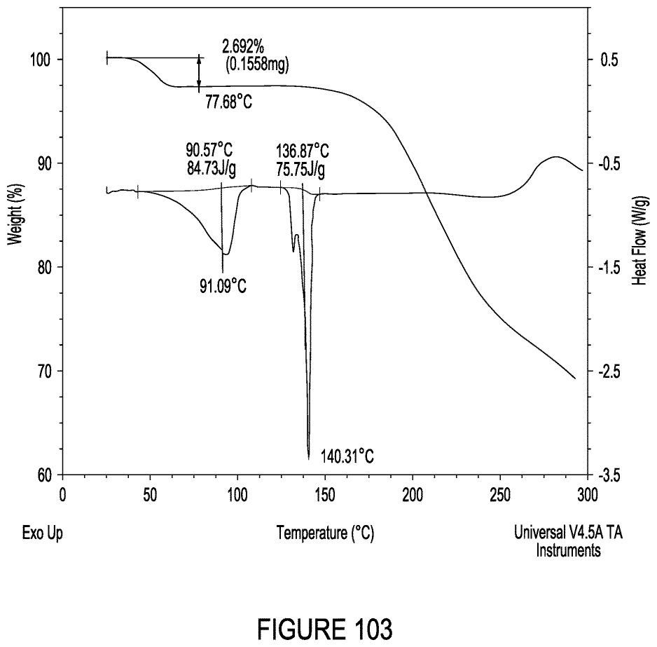

In some embodiments, the disclosure provides Compound A free base in amorphous form. In some embodiments, the disclosure provides amorphous form Compound A free base characterized by mDSC as set forth in the Examples and substantially as shown in FIG. 103. In some embodiments, amorphous form Compound A free base has a glass transition temperature (Tg) of 115° C. to 125° C., by way of example, a Tg that is 118° C.±3° C.

Amorphous form Compound A free base can be formed upon cooling of a melt of Compound A free base under suitable conditions. For example, in some embodiments Compound A free base Form 1 can be heated at a rate of 20° C./min to a temperature of 300° C. and melted in a hot stage microscope, upon which a sample can be removed and rapidly cooled to room temperature.

### Crystalline Solvates

In some embodiments, the disclosure provides solvates and hydrates of crystalline Compound A. Typically, the solvates and hydrates are formed from mixtures comprising Compound A and water and/or one or more organic solvents (e.g., by slurring or evaporation). Nonlimiting examples of suitable organic solvents for forming solvates of Compound A include ethanol, isopropanol, methanol, and acetonitrile. For example, ethanol and isopropanol solvates of Compound A disclosed herein can be obtained via slow evaporation or slurring of mixtures comprising Compound A and ethanol and/or isopropanol.

### Crystalline Compound A Free Base Hydrate (“Form 8”)

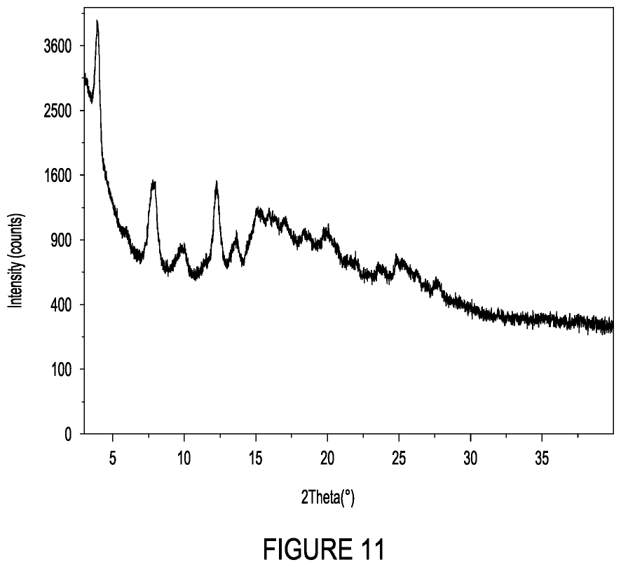

In some embodiments, the disclosure provides a crystalline form of Compound A free base hydrate (“Form 8”) characterized by an XRPD pattern comprising peaks at 4.0, 7.7, 8.0, 12.3, and 15.1±0.2° 2θ using CuKα radiation. In some embodiments, Form 8 is further characterized by XRPD pattern peaks at 6.1, 10.0, 13.7, 17.0, 18.4, and 19.9±0.2° 2θ using CuKα radiation. In some embodiments, Form 8 has an XRPD pattern substantially as shown in FIG. 11.

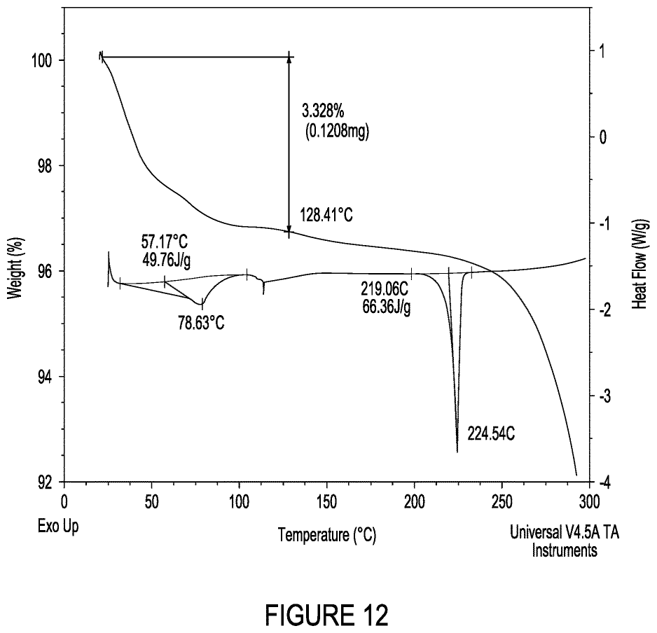

Alternatively, or in addition to, Form 8 can be characterized using DSC. In some embodiments, Form 8 has a DSC substantially as shown in FIG. 12. Differential scanning calorimetry (DSC) thermographs were obtained for Form 8, as set forth in the Examples.

Alternatively, or in addition to, Form 8 can be characterized using thermogravimetric analysis (TGA). In some embodiments, Compound A free base Form 8 has a TGA substantially as shown in FIG. 12.

As shown in FIG. 12, Form 8 exhibits endothermic events with onset temperatures of 57° C. and 219° C. and peak temperatures of 79° C. and 225° C., respectively. Moreover, Form 8 exhibited a weight loss of 3.3% as determined by TGA up to 128° C.

### Crystalline Compound A Ethanol Solvate (“Form 2A”)

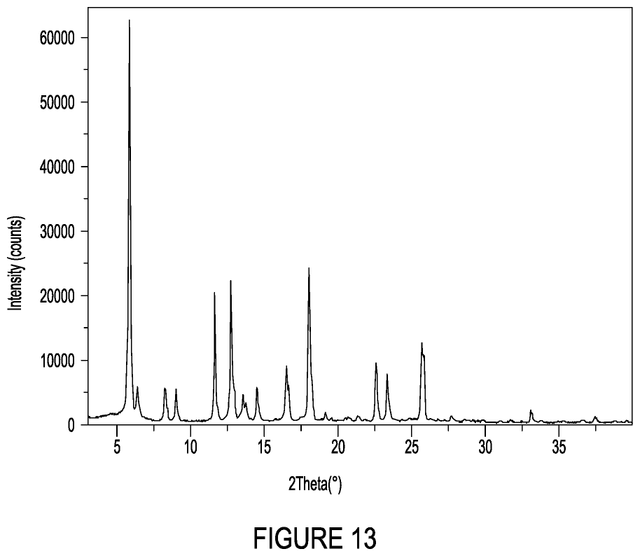

In some embodiments, the disclosure provides crystalline Compound A ethanol solvate (“Form 2A”) characterized by an X-ray powder diffraction (XRPD) pattern comprising peaks at 5.8, 11.6, 12.7, 18.0, and 25.7±0.2° 2θ using CuKα radiation. In some embodiments, Form 2A is further characterized by XRPD pattern peaks at 16.5, 22.6, 23.3, and 25.8±0.2° 2θ using CuKα radiation. In some embodiments, Form 2A has an XRPD pattern substantially as shown in FIG. 13.

In some embodiments, the disclosure provides Form 2A having unit cell dimensions of a=14.0551 (3) Å; b=5.4000 (10) Å; c=14.9397 (3) Å; α=90°; β=95.5093 (8)°; and γ=90°.

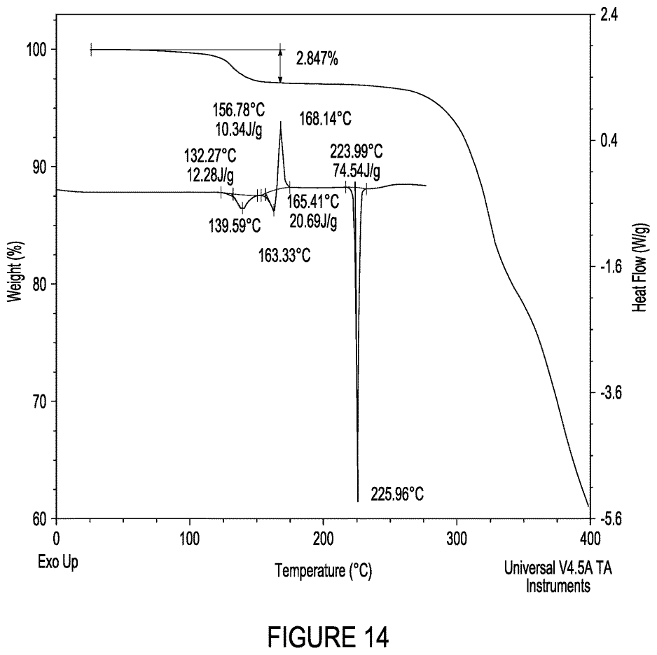

Alternatively, or in addition to, Form 2A can be characterized using DSC. In some embodiments, Form 2A has a DSC substantially as shown in FIG. 14. Differential scanning calorimetry (DSC) thermographs were obtained for Form 2A, as set forth in the Examples.

Alternatively, or in addition to, Form 2A can be characterized using thermogravimetric analysis (TGA). In some embodiments, Compound A free base Form 2A has a TGA substantially as shown in FIG. 14.

As shown in FIG. 14, the DSC curve of Form 2A shows the first small endotherm with an extrapolated onset at 132° C. as a result of desolvation of ethanol, and the second endotherm overlaps the exotherm with subsequent recrystallization and was followed by an extrapolated melting onset at 224° C. and an endothermic transition of melting at 218° C. to 236° C. In some embodiments, the extrapolated onset is at 224° C.±3° C. All of the melting onsets of solvate/hydrate are close to the melting onset of Free Base Form 1. The DSC heating process may result in re-crystallization of solvate/hydrate to Free Base Form 1.

### Crystalline Compound A Isopropanol Solvate (“Form 3A”)

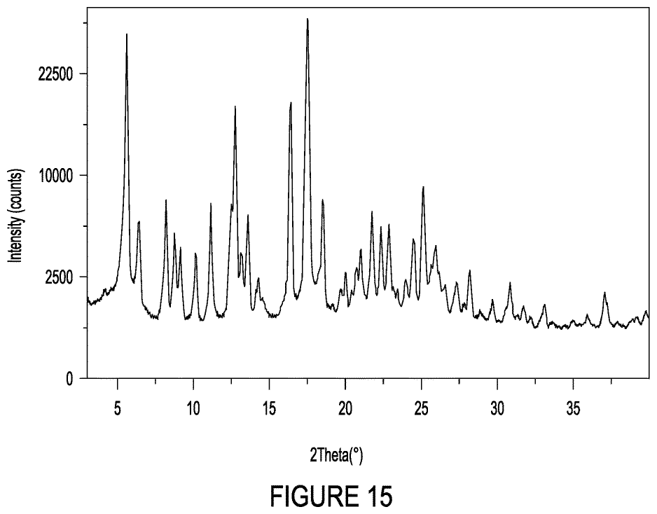

In some embodiments, the disclosure provides crystalline Compound A isopropanol solvate (“Form 3A”) characterized by an X-ray powder diffraction (XRPD) pattern comprising peaks at 5.6, 12.8, 16.4, 17.5, and 25.1±0.2° 2θ using CuKα radiation. In some embodiments, the XRPD of Form 3A is further characterized by XRPD pattern peaks at 8.2, 11.2, 12.5, 18.6, and 21.8±0.2° 2θ using CuKα radiatin. In some embodiments, the XRPD of Form 3A is further characterized by XRPD pattern peaks at 6.4, 8.8, 9.2, 13.6, 21.0, 22.4, 22.9, 24.5, and 25.9±0.2° 2θ using CuKα radiation. In some embodiments, Form 3A has an XRPD pattern substantially as shown in FIG. 15.

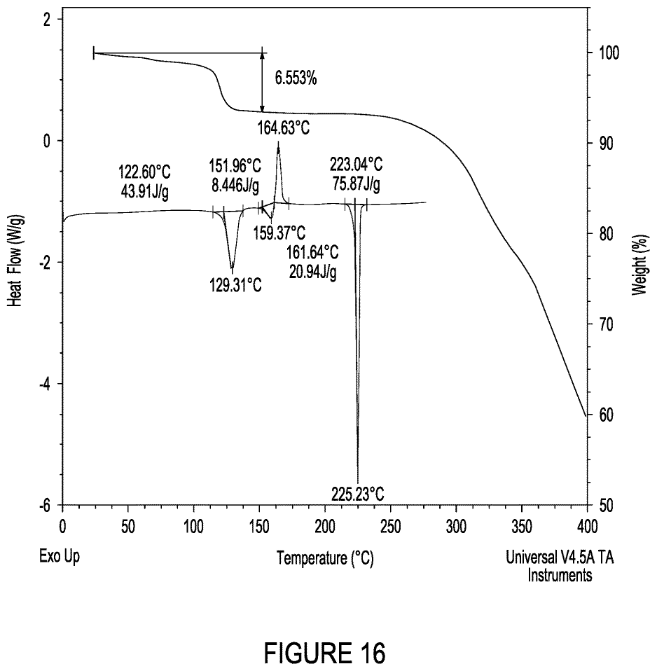

Alternatively, or in addition to, Form 3A can be characterized using DSC. In some embodiments, Form 3A has a DSC substantially as shown in FIG. 16. Differential scanning calorimetry (DSC) thermographs were obtained for Form 3A, as set forth in the Examples.

Alternatively, or in addition to, Form 3A can be characterized using thermogravimetric analysis (TGA). In some embodiments, Compound A free base Form 3A has a TGA substantially as shown in FIG. 16.

In some embodiments, Form 3A shows the first small endotherm with an extrapolated onset at 122.6° C. as a result of desolvation of isopropyl alcohol (IPA), and the second endotherm overlapped the exotherm with subsequent recrystallization and followed by an extrapolated melting onset at 223° C.±3° C. In some embodiments, the DSC of Form 3A has an endothermic onset transition is at 223° C.±3° C.

### Crystalline Compound A Acetone Solvate (“Form 4A”)

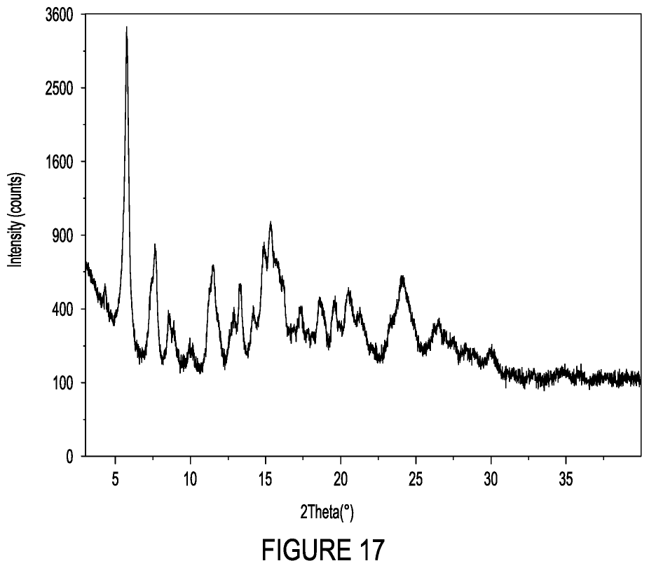

In some embodiments, the disclosure provides crystalline Compound A acetone solvate (“Form 4A”) characterized by an X-ray powder diffraction (XRPD) pattern comprising peaks at 5.7, 7.7, 11.5, 14.8, and 15.3±0.2° 2θ using CuKα radiation. In some embodiments, Form 4A is further characterized by XRPD pattern peaks at 4.3, 7.3, 13.4, 16.2, and 24.0±0.2° 2θ using CuKα radiation. In some embodiments, Form 4A is also characterized by XRPD pattern peaks at 11.2, 18.5, 19.6, and 20.4±0.2° 2θ using CuKα radiation. In some embodiments, Form 4A has an XRPD pattern substantially as shown in FIG. 17.

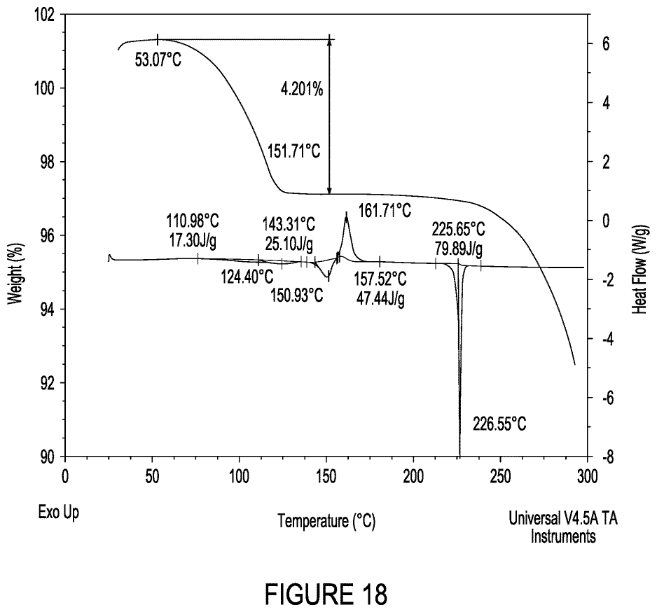

Alternatively, or in addition to, Form 4A can be characterized using DSC. In some embodiments, Form 4A has a DSC substantially as shown in FIG. 18. Differential scanning calorimetry (DSC) thermographs were obtained for Form 4A, as set forth in the Examples.

Alternatively, or in addition to, Form 4A can be characterized using thermogravimetric analysis (TGA). In some embodiments, Compound A free base Form 4A has a TGA substantially as shown in FIG. 18.

### Crystalline Compound A Free Base Methanol Solvate (“Form 5A”)

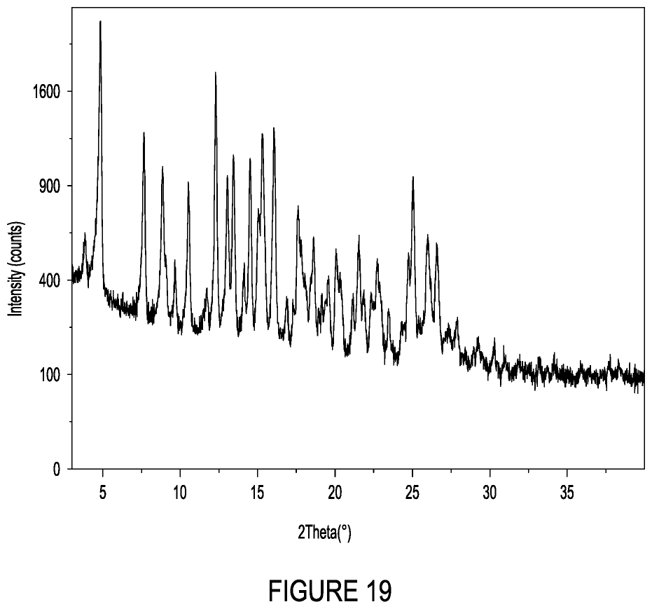

In some embodiments, the disclosure provides crystalline Compound A methanol solvate (“Form 5A”) characterized by an X-ray powder diffraction (XRPD) pattern comprising peaks 4.8, 7.7, 12.3, 15.3, and 16.1±0.2° 2θ using CuKα radiation. In some embodiments, Form 5A is further characterized by XRPD pattern peaks at 8.9, 10.5, 13.1, 13.4, 14.5, 17.6, 21.5, and 25.0±0.2° 2θ using CuKα radiation. In some embodiments, Form 5A is also characterized by XRPD pattern peaks at 9.7, 14.1, 18.7, 19.6, 22.7, 24.7, 26.0, and 26.6±0.2° 2θ using CuKα radiation. In some embodiments, Form 5A has an XRPD pattern substantially as shown in FIG. 19.

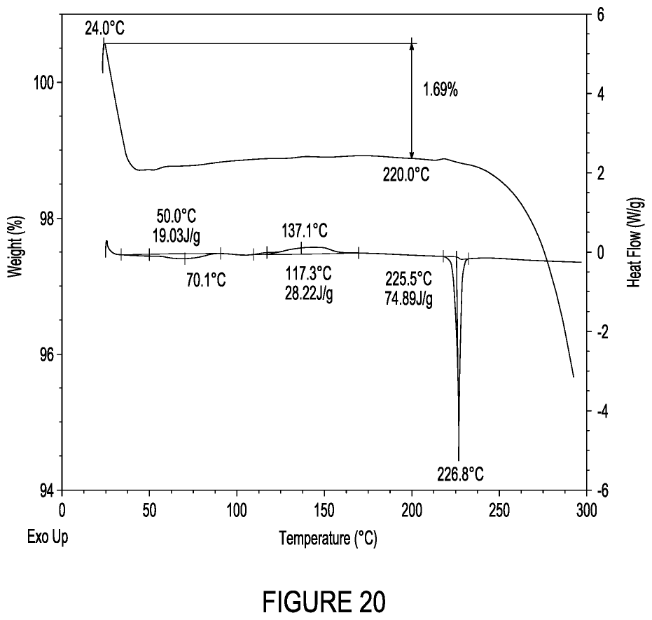

Alternatively, or in addition to, Form 5A can be characterized using DSC. In some embodiments, Form 5A has a DSC substantially as shown in FIG. 20. Differential scanning calorimetry (DSC) thermographs were obtained for Form 5A, as set forth in the Examples.

Alternatively, or in addition to, Form 5A can be characterized using thermogravimetric analysis (TGA). In some embodiments, Compound A free base Form 5A has a TGA substantially as shown in FIG. 20.

Crystalline Compound A Free Base Methyl-Tetrahydrofuran Solvate (“Form 6A”) In some embodiments, the disclosure provides crystalline Compound A methyl tetrahydrofuran solvate (“Form 6A”) characterized by a XRPD pattern comprising peaks at 7.6, 11.3, 15.1, 18.3, and 28.0±0.2° 2θ using CuKα radiation. In some embodiments, Form 6A is further characterized by XRPD pattern peaks at 12.4, 15.7, 17.3, 17.7, 18.8, 19.7, 21.3, 22.7, 25.8, 26.1, and 26.5±0.2° 2θ using CuKα radiation. In some embodiments, Form 6A is also characterized by XRPD pattern peaks at 13.6, 14.2, 16.5, 16.9, 20.5, 26.9, and 28.4±0.2° 2θ using CuKα radiation.

In some embodiments, the disclosure provides crystalline salt forms of Compound A. Compound A has ionizable functional groups with one weakly basic pKa value of 4.27 suitable for salt formation. Suitable nonlimiting examples of counterions for forming salts with Compound A include benzene sulfonic acid (BSA), toluene sulfonic acid (TSA), sulfuric acid, hydrochloric acid, malonic acid, naphthalene-2-sulfonic acid, methane sulfonic acid, oxalic acid, tartaric acid, ethane sulfonic acid, cyclamic acid, maleic acid, or phosphoric acid.

### Crystalline Compound A Toluene Sulfonate (“Form A1”)

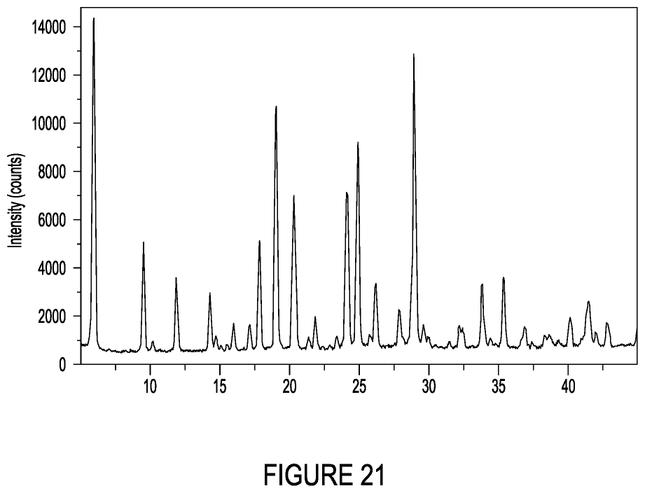

In some embodiments, the crystalline form of Compound A toluene sulfonate salt (“Form A1”), is characterized by an X-ray powder diffraction (XRPD) pattern comprising peaks at 6.0, 19.1, 20.3, 24.1, 24.9, and 28.9±0.2° 2θ using CuKα radiation. In some embodiments, the XRPD Form A1 is further characterized by peaks at 9.5, 11.9, 14.3, 17.9, 26.2, 33.8, and 35.4±0.2° 2θ using CuKα radiation. Alternatively, or in addition to, in some embodiments, Form A1 disclosed herein has an XRPD pattern substantially as shown in FIG. 21.

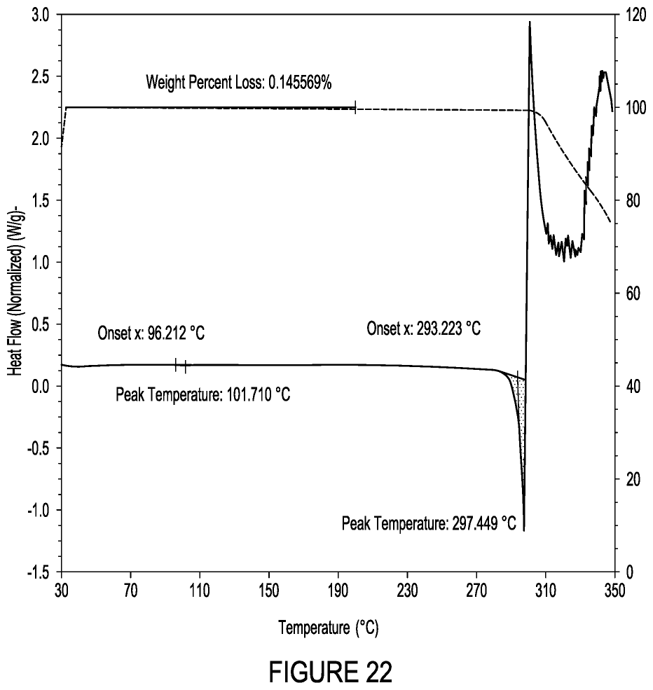

Alternatively, or in addition to, Form A1 can be characterized using DSC and/or TGA. In some embodiments, Form A1 has a DSC substantially as shown in FIG. 22. As shown in FIG. 22, Form A1 has an endothermic transition at 290° C. to 300° C., as measured by DSC. In some embodiments, the extrapolated onset is at at 293° C.±3° C., due to endothermic melt and/or decomposition.

In some embodiments, Form A1 has a TGA curve substantially as shown in FIG. 22.

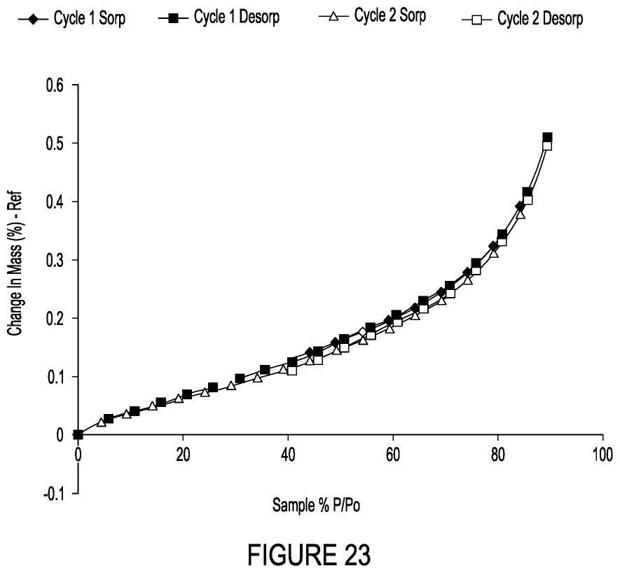

In some embodiments, Form A1 has a DVS substantially as shown in FIG. 23.

### Crystalline Compound A Toluene Sulfonate (“Form A2”)

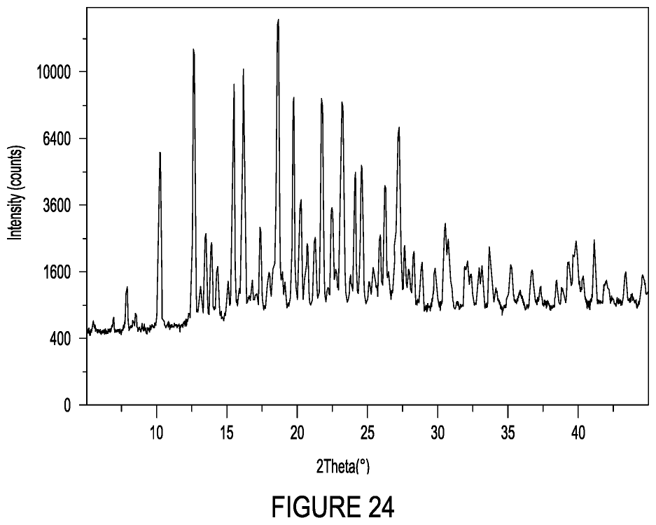

In some embodiments, the disclosure provides a crystalline Compound A toluene sulfonate salt (“Form A2”), characterized by an X-ray powder diffraction (XRPD) pattern comprising peaks at 12.7, 15.5, 16.2, 18.7, 19.7, and 21.8±0.2° 2θ using CuKα radiation. In some embodiments, Form A2 is characterized by an X-ray powder diffraction (XRPD) pattern comprising peaks at 10.2, 23.2, 24.1, 24.6, 26.3, and 27.2±0.2° 2θ using CuKα radiation. Alternatively, or in addition to, in some embodiments the XRPD pattern of Form A2 is as shown in FIG. 24.

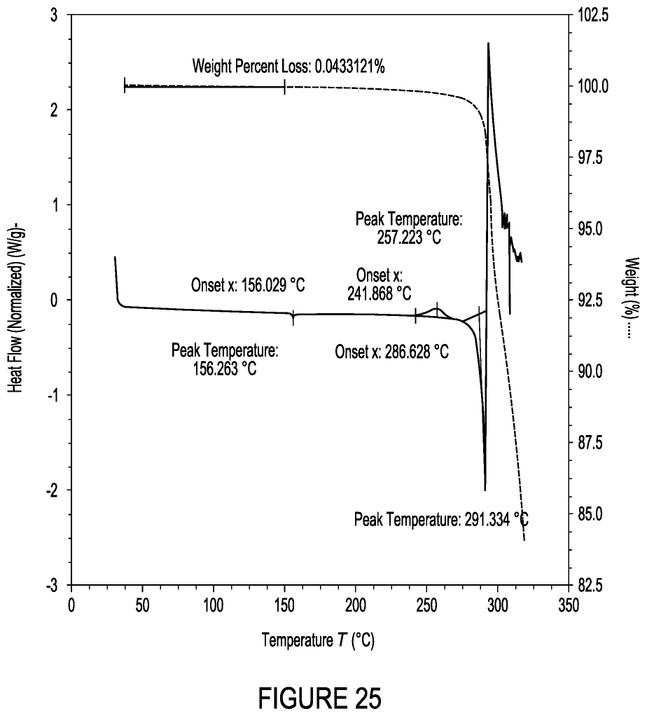

Alternatively, or in addition to, Form A2 can be characterized using DSC and/or TGA. In some embodiments, Form A2 has a DSC substantially as shown in FIG. 25. As shown in FIG. 25, Form A2 shows an exotherm with an extrapolated onset at 241.9° C. due to recrystallization and followed by an extrapolated melt/decomposition at 287° C. Form A2 has an endothermic transition at 270° C. to 300° C., due to endothermic melt or decomposition, as measured by DSC. In some embodiments, the extrapolated onset is at 287° C.±3° C.

In some embodiments, Form A2 has a TGA curve substantially as shown in FIG. 25.

### Crystalline Compound A Toluene Sulfonate (“Form A3”)

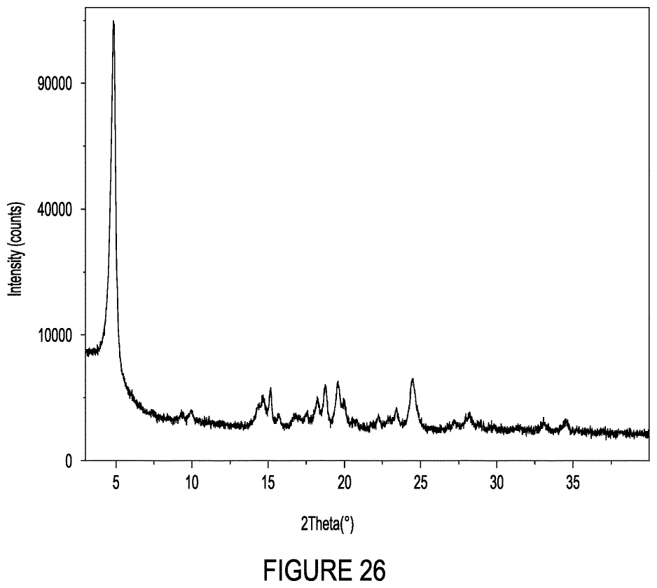

In some embodiments, the disclosure provides a crystalline Compound A toluene sulfonate salt (“Form A3”), characterized by an X-ray powder diffraction (XRPD) pattern comprising peaks at 4.9, 15.2, 18.8, 19.5, and 24.5±0.2° 2θ using CuKα radiation. Alternatively, or in addition to, in some embodiments the XRPD pattern of Form A3 is as shown in FIG. 26.

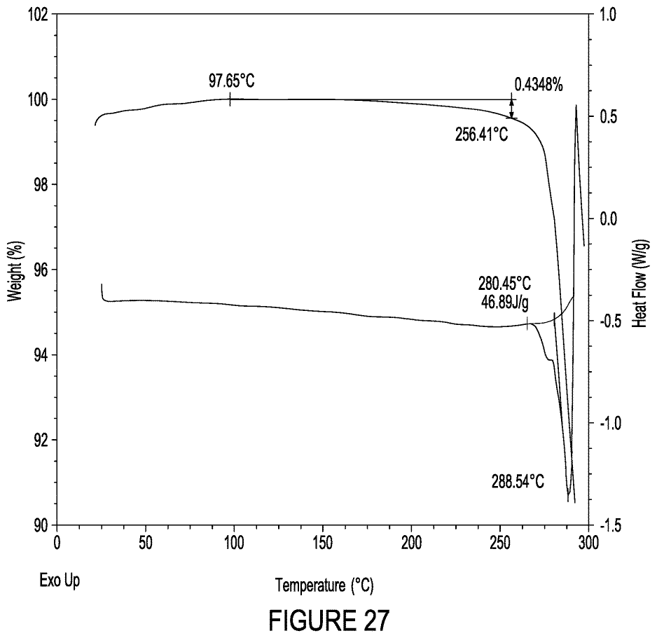

Alternatively, or in addition to, Form A3 can be characterized using DSC, TGA, and/or DVS. In some embodiments, Form A3 has a DSC substantially as shown in FIG. 27. In some embodiments, Form A3 has a TGA as shown in FIG. 27.

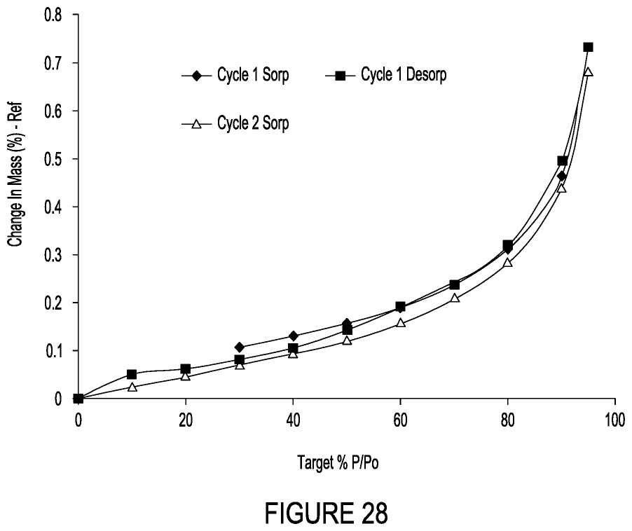

In addition, Form A3 can be characterized using DVS. In some embodiments, the disclosure provides Form A3 having a DVS as shown in FIG. 28.

### Crystalline Compound A Benzene Sulfonate (“Form B1”)

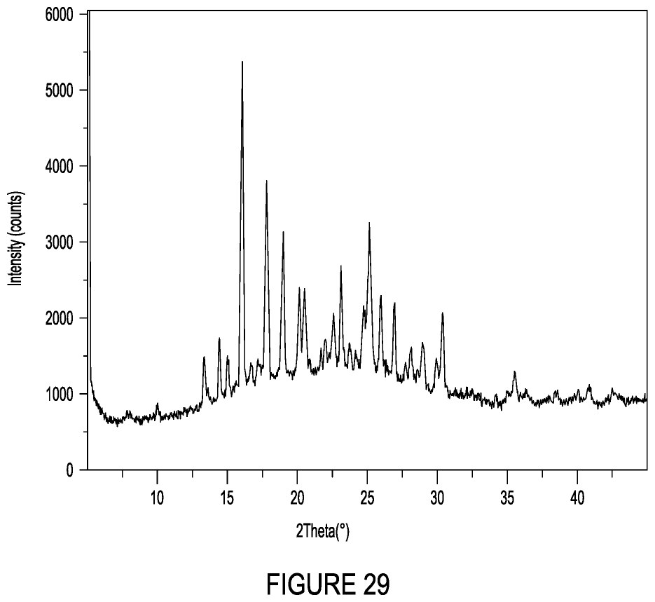

In some embodiments, the disclosure provides a crystalline form of Compound A benzene sulfonate salt (“Form B1”), characterized by an X-ray powder diffraction (XRPD) pattern comprising peaks at 5.1, 16.1, 17.9, 19.0, and 25.2±0.2° 2θ using CuKα radiation. In some embodiments, Form B1 is further characterized by XRPD pattern peaks at 20.2, 20.6, 23.2, 26.0, 27.0, and 30.4±0.2° 2θ using CuKα radiation. In some embodiments, Form B1 has an XRPD pattern substantially as shown in FIG. 29.

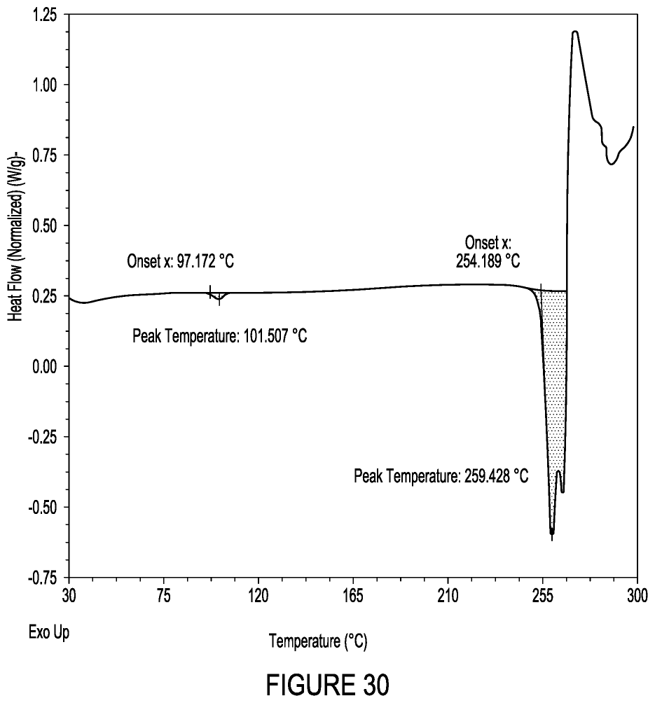

In some embodiments, Form B1 DSC shows an endotherm with an extrapolated onset at 97° C. followed by an extrapolated endothermic melt/decomposition at 254° C. Form B1 has an endothermic transition at 240° C. to 260° C., as measured by DSC. In some embodiments, the extrapolated onset is at 254° C.±3° C., due to endothermic melt and/or decomposition. In some embodiments, Form B1 has a DSC substantially as shown in FIG. 30.

### Crystalline Compound A Chloride (“Form C1”)

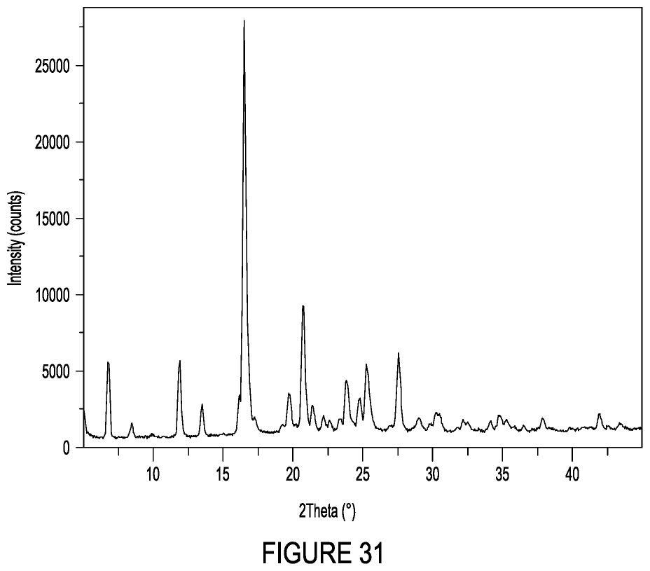

In some embodiments, the crystalline form of Compound A chloride salt (“Form C1”) is characterized by an X-ray powder diffraction (XRPD) pattern comprising peaks at 6.8, 11.9, 16.6, 20.7, 23.8, 25.3, and 27.6±0.2° 2θ using CuKα radiation. In some embodiments, the crystalline Form C1 of Compound A chloride salt has an XRPD pattern substantially as shown in FIG. 31.

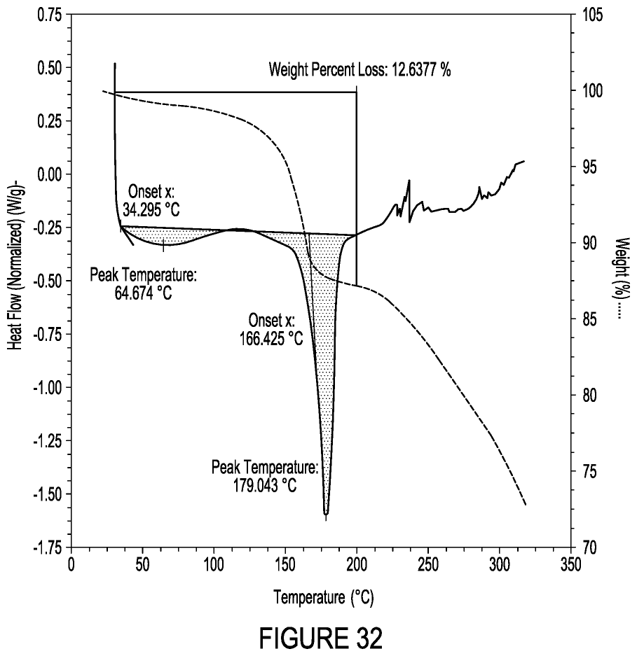

In some embodiments, Form C1 has a DSC substantially as shown in FIG. 32. As shown in FIG. 32, the DSC of Form C1 shows a first broad endotherm with an extrapolated onset at 34° C. and first endothermic transition at 30° C. to 110° C. An extrapolated endothermic melt/decomposition/salt disproportionation at 166.4° C. In some embodiments, the extrapolated onset is at 34° C.±3° C. In some embodiments, Form C1 is further characterized by a second endothermic transition at 160° C. to 170° C., as measured by DSC. In some embodiments, the second extrapolated onset is at 166° C.±3° C. due to endothermic melt and/or decomposition.

The crystalline Compound A chloride salt Form C1 can be characterized by TGA. In some embodiments, the disclosed Form C1 has a TGA substantially as shown in FIG. 32.

### Crystalline Compound A Chloride (“Form C2”)

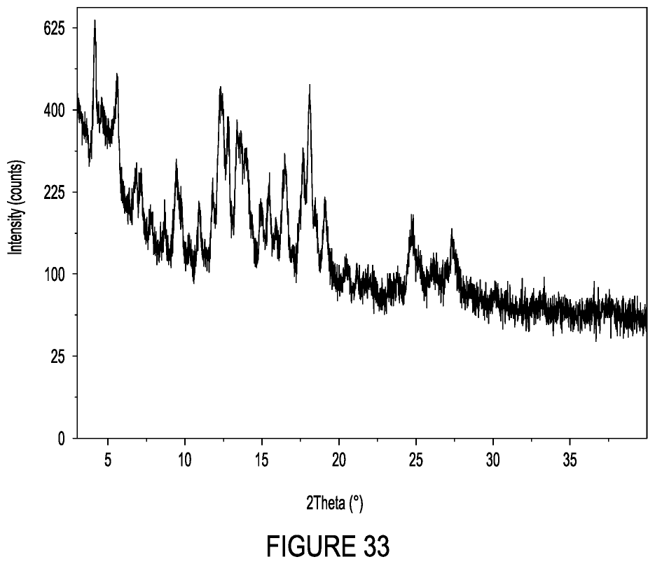

In some embodiments, the crystalline form of Compound A chloride salt (“Form C2”) is characterized by an X-ray powder diffraction (XRPD) pattern comprising peaks at 4.2, 5.6, 12.2, 12.9, and 18.1=0.2° 2θ using CuKα radiation. In some embodiments, Form C2 is further characterized by an X-ray powder diffraction (XRPD) pattern comprising peaks at 6.8, 7.1, 9.4, 13.4, 14.0, 16.5, and 17.7±0.2° 2θ using CuKα radiation. In some embodiments, Form C2 is also characterized by an X-ray powder diffraction (XRPD) pattern comprising peaks at 7.8, 8.7, 10.9, 11.8, 14.9, 15.4, and 19.1±0.2° 2θ using CuKα radiation. In some embodiments, disclosure provides crystalline form of Compound A chloride salt (“Form C2”) having an XRPD pattern substantially as shown in FIG. 33.

Alternatively, or in addition to, Form C2 can be characterized using DSC, TGA, and or DVS.

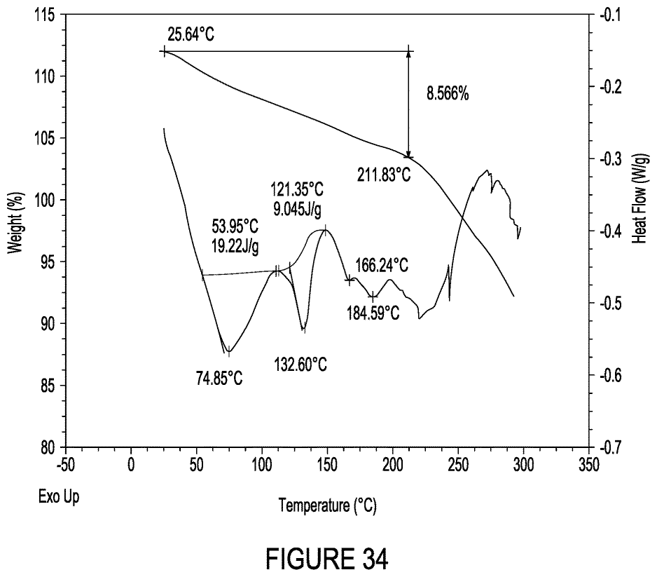

In some embodiments, the disclosure provides Form C2 having a DSC substantially as shown in FIG. 34. As shown in FIG. 34, the DSC of Form C2 shows a first broad endotherm with an extrapolated onset at 34° C. and first endothermic transition at 30° C. to 110° C. An extrapolated endothermic melt/decomposition/salt disproportionation at 166° C. In some embodiments, the extrapolated onset is at 34° C.±3° C. In some embodiments, Form C2 is further characterized by a second endothermic transition at 160° C. to 170° C., as measured by DSC. In some embodiments, the second extrapolated onset is at 166° C.±3° C. due to endothermic melt and/or decomposition.

In some embodiments, the disclosed Form C2 has a TGA substantially as shown in FIG. 34.

### Crystalline Compound A Sulfate (“Form D1”)

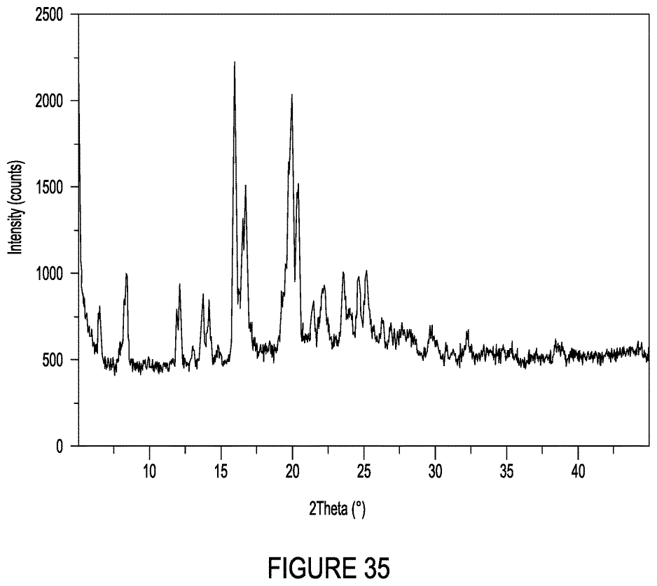

In some embodiments, the disclosure provides a crystalline Compound A sulfonate (“Form D1”), characterized by an X-ray powder diffraction (XRPD) pattern comprising peaks at 16.0, 16.5, 16.7, 20.0, and 20.4±0.2° 2θ using CuKα radiation. In some embodiments Form D1 is further characterized by XRPD pattern peaks at 8.4, 12.1, 13.8, 14.2, 23.6, 24.6, and 25.2±0.2° 2θ using CuKα radiation. In some embodiments, the disclosure provides Form D1 salt having an XRPD substantially as shown in FIG. 35.

### Crystalline Compound A Malonate (“Form E1”)

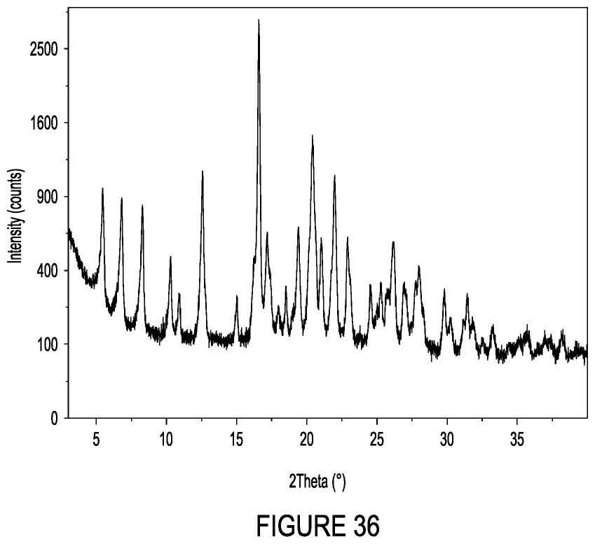

In some embodiments, the disclosure provides a crystalline Compound A malonate salt (“Form E1”), characterized by an X-ray powder diffraction (XRPD) pattern comprising peaks at 6.8, 12.6, 16.6, 20.4, and 22.0±0.2° 2θ using CuKα radiation. In some embodiments, Form E1 is further characterized by by XRPD pattern peaks at 5.4, 8.3, 17.2, 19.4, 21.0, 22.9, and 26.2±0.2° 2θ using CuKα radiation. In some embodiments, the disclosure provides crystalline Compound A malonate Form E1 having an XRPD substantially as shown in FIG. 36.

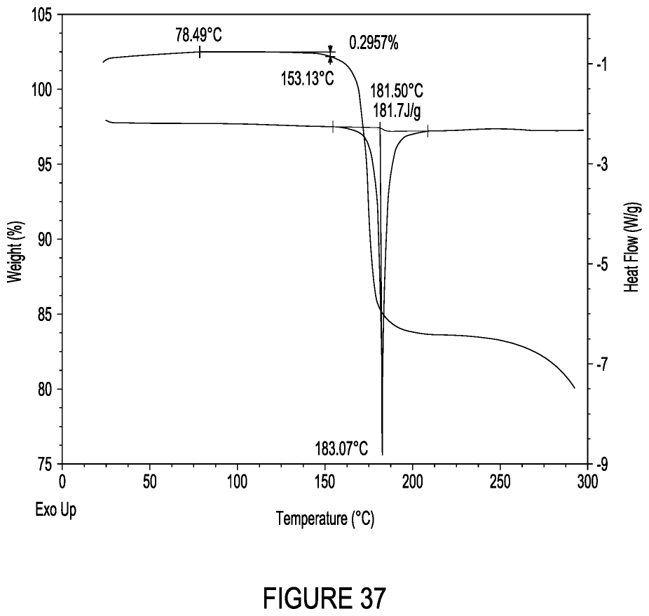

In some embodiments, Form E1 is characterized by DSC, TGA, and/or DVS. In some embodiments, the disclosure provides Form E1 having a DSC substantially as shown in FIG. 37.

In some embodiments, the disclosure provides Form E1 having a TGA substantially as shown in FIG. 37.

### Crystalline Compound A Naphthalene-2-Sulfonate (“Form F1”)

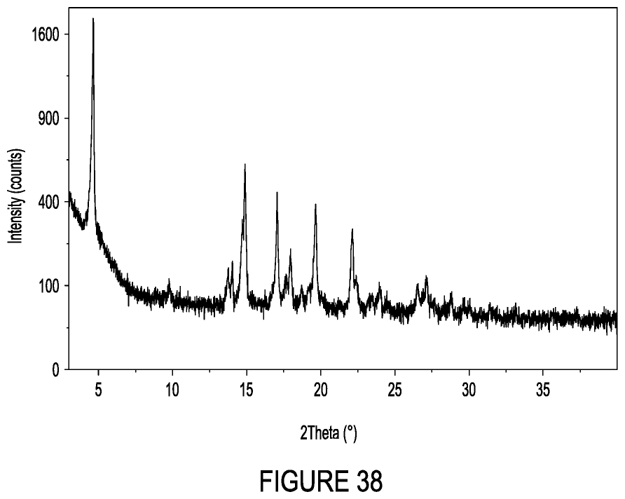

In some embodiments, the disclosure provides a crystalline Compound A naphthalene-2-sulfonate salt (“Form F1”), characterized by an X-ray powder diffraction (XRPD) pattern comprising peaks at 4.7, 14.7, 14.9, 17.0, 19.6, and 22.1±0.2° 2θ using CuKα radiation. In some embodiments, the disclosure provides crystalline Compound A naphthalene-2-sulfonate Form F1 having an XRPD substantially as shown in FIG. 38.

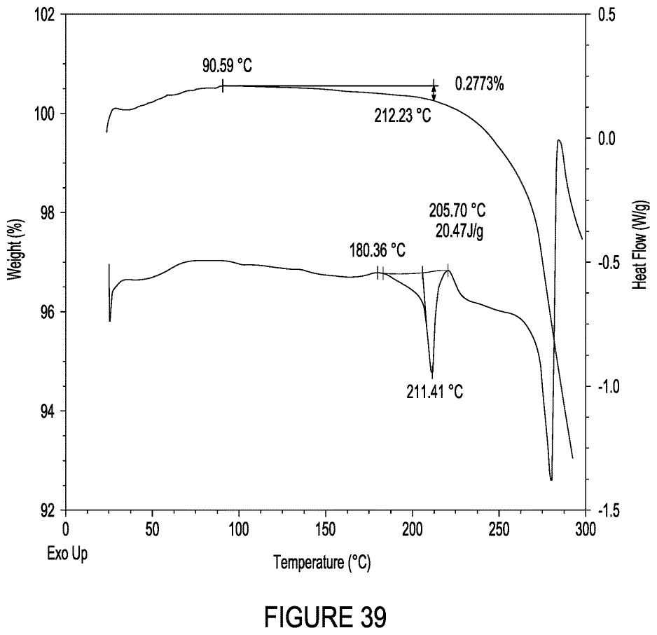

In some embodiments, Form F1 is characterized by DSC, TGA, and/or DVS. In some embodiments, the disclosure provides Form F1 having a DSC substantially as shown in FIG. 39.

In some embodiments, the disclosure provides Form F1 having a TGA substantially as shown in FIG. 39.

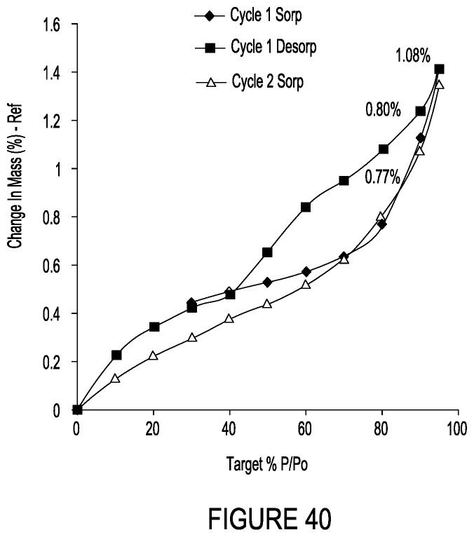

In some embodiments, the disclosure provides Form F1 having a DVS substantially as shown in FIG. 40.

### Crystalline Compound A Naphthalene-2-Sulfonate (“Form F2”)

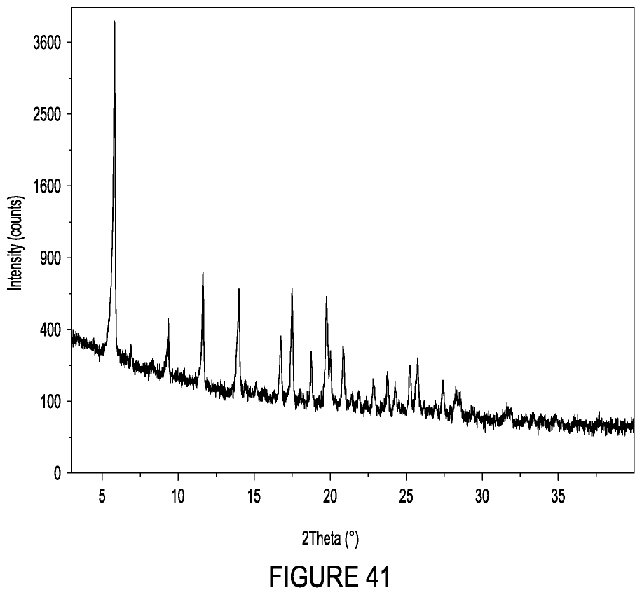

In some embodiments, the disclosure provides a crystalline Compound A naphthalene-2-sulfonate salt (“Form F2”), characterized by an X-ray powder diffraction (XRPD) pattern comprising peaks at 5.8, 11.6, 14.0, 17.5, and 19.7±0.2° 2θ using CuKα radiation. In some embodiments, the disclosure provides crystalline Compound A naphthalene-2-sulfonate Form F2 having an XRPD substantially as shown in FIG. 41.

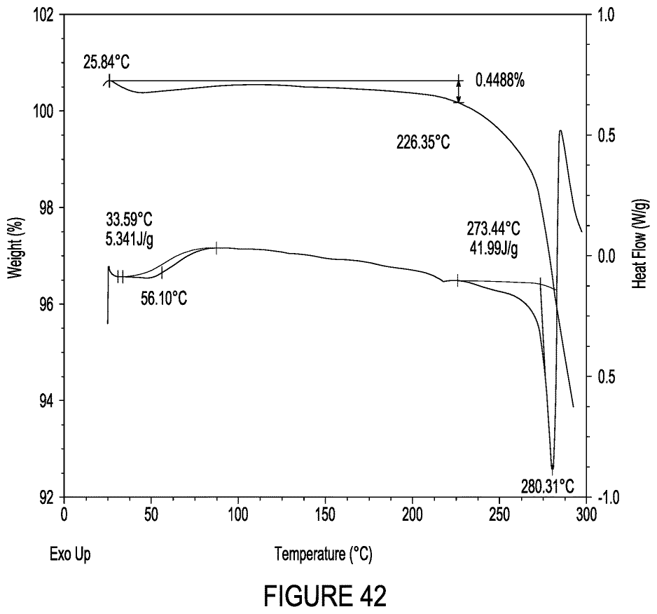

In some embodiments, Form F2 is characterized by DSC, TGA, and/or DVS. In some embodiments, the disclosure provides Form F2 having a DSC substantially as shown in FIG. 42.

In some embodiments, the disclosure provides Form F2 having a TGA substantially as shown in FIG. 42.

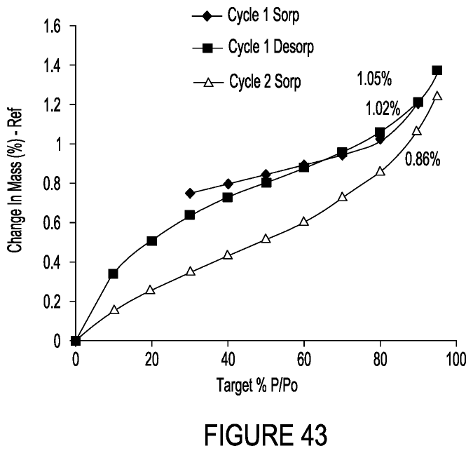

In some embodiments, the disclosure provides Form F2 having a DVS substantially as shown in FIG. 43.

### Crystalline Compound A Naphthalene-2-Sulfonate (“Form F3”)

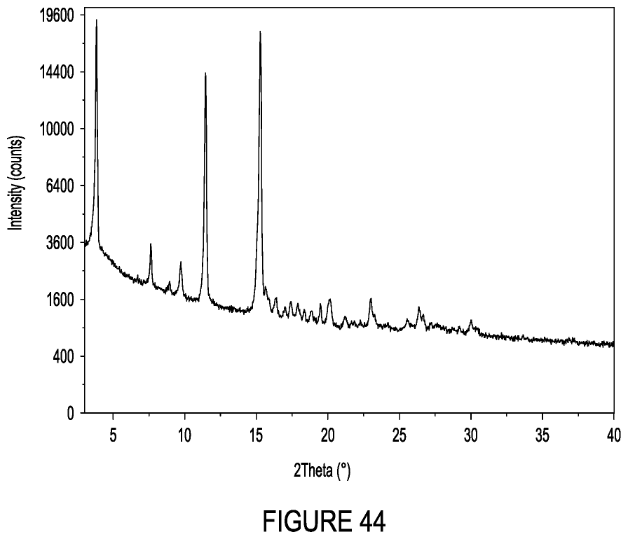

In some embodiments, the disclosure provides a crystalline Compound A naphthalene-2-sulfonate salt (“Form F3”), characterized by an X-ray powder diffraction (XRPD) pattern comprising peaks at at 3.8, 7.6, 9.7, 11.5, and 15.3±0.2° 2θ using CuKα radiation. In some embodiments, the disclosure provides crystalline Compound A naphthalene-2-sulfonate Form F3 having an XRPD substantially as shown in FIG. 44.

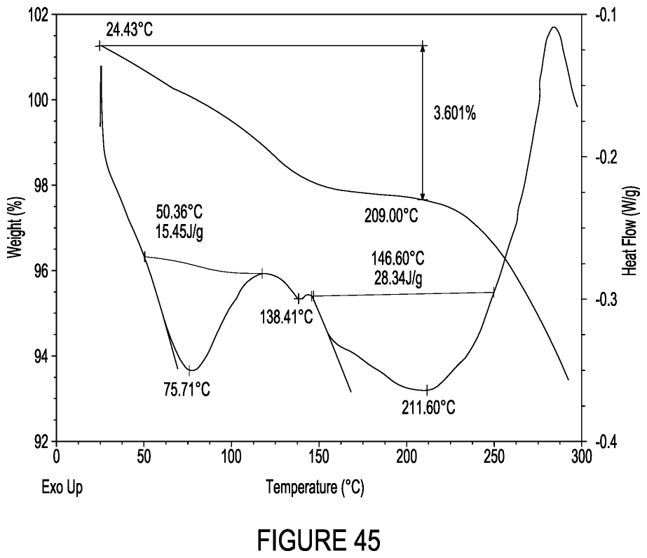

In some embodiments, Form F3 is characterized by DSC, TGA, and/or DVS. In some embodiments, the disclosure provides Form F3 having a DSC substantially as shown in FIG. 45.

In some embodiments, the disclosure provides Form F3 having a TGA substantially as shown in FIG. 45.

### Crystalline Compound A Methane Sulfonate (“Form G1”)

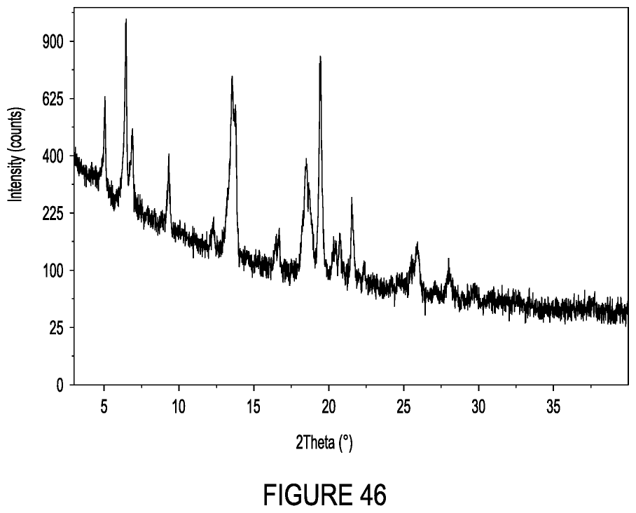

In some embodiments, the disclosure provides a crystalline Compound A methane sulfonate salt (“Form G1”), characterized by an X-ray powder diffraction (XRPD) pattern comprising peaks at 5.1, 6.5, 13.6, 13.8, and 19.5±0.2° 2θ using CuKα radiation. In some embodiments, Form G1 is further characterized by XRPD pattern peaks at 6.9, 9.3, 18.5, 20.8, and 21.5±0.2° 2θ using CuKα radiation. In some embodiments, the disclosure provides crystalline Compound A methane sulfonate salt Form G1 having an XRPD substantially as shown in FIG. 46.

### Crystalline Compound A Methane Sulfonate (“Form G2”)

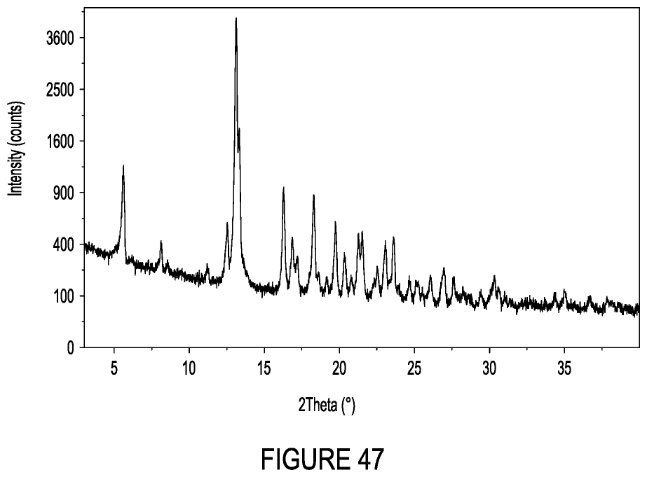

In some embodiments, the disclosure provides a crystalline Compound A methane sulfonate salt (“Form G2”), characterized by an X-ray powder diffraction (XRPD) pattern comprising peaks at 5.6, 13.1, 13.3, 16.3, and 18.3±0.2° 2θ using CuKα radiation. In some embodiments, Form G2 is further characterized by an X-ray powder diffraction (XRPD) pattern comprising peaks at 12.5, 19.8, 21.5, and 9.4±0.2° 2θ using CuKα radiation. In some embodiments, the disclosure provides crystalline Compound A methane sulfonate salt Form G2 having an XRPD substantially as shown in FIG. 47.

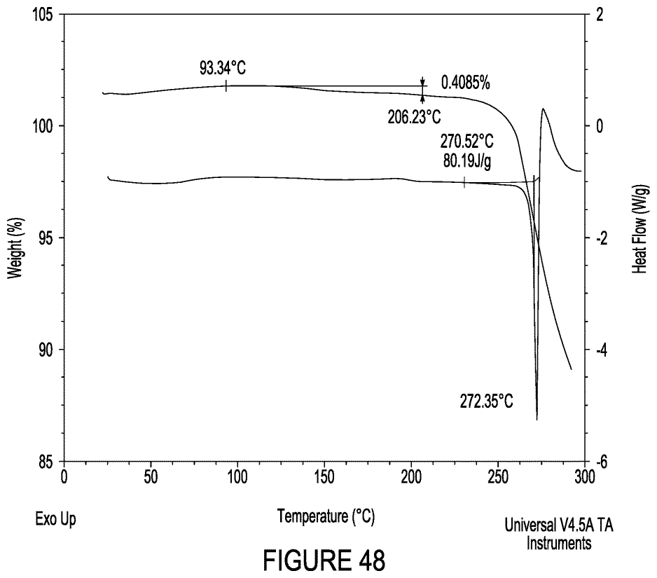

Form G2 can be characterized by DSC, TGA, and/or DVS. In some embodiments, the disclosure provides crystalline Compound A methane sulfonate salt Form G2 having a DSC substantially as shown in FIG. 48.

In some embodiments, the disclosure provides crystalline Compound A methane sulfonate salt Form G2 having a TGA substantially as shown in FIG. 48.

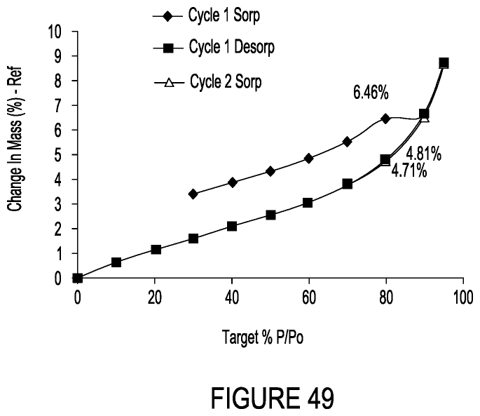

In some embodiments, the disclosure provides crystalline Compound A methane sulfonate salt Form G2 having a DVS profile substantially as shown in FIG. 49.

### Crystalline Compound A Oxalate (“Form H2”)

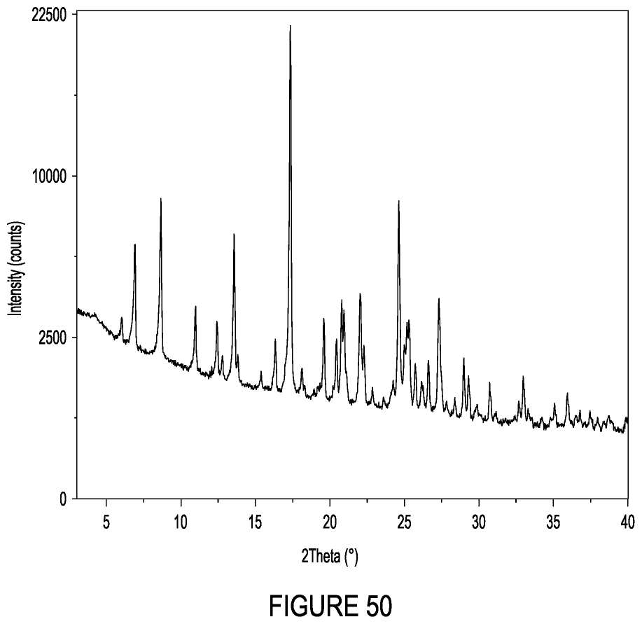

In some embodiments, the disclosure provides a crystalline Compound A oxalate salt (“Form H2”), characterized by an X-ray powder diffraction (XRPD) pattern comprising peaks at 6.9, 8.7, 13.6, 17.4, and 24.6±0.2° 2θ using CuKα radiation. In some embodiments, Form H2 is further characterized by XRPD pattern peaks at 11.0, 19.6, 20.8, 21.0, 22.0, 25.3, and 27.3±0.2° 2θ using CuKα radiation. In some embodiments, the disclosure provides crystalline form of Compound A oxalate salt Form H2 having an XRPD substantially as shown in FIG. 50.

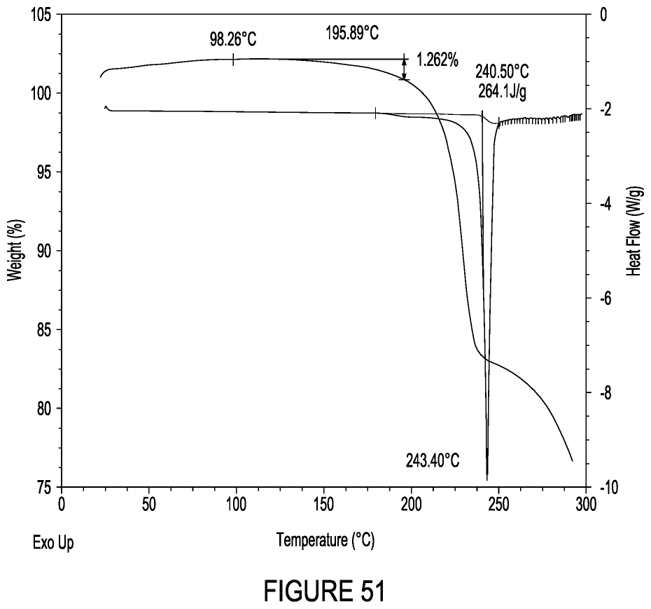

Form H2 can be characterized by DSC, TGA, and/or DVS. In some embodiments, the disclosure provides crystalline Compound A oxalate salt Form H2 having a DSC substantially as shown in FIG. 51.

In some embodiments, the disclosure provides crystalline Compound A oxalate salt Form H2 having a TGA substantially as shown in FIG. 51.

### Crystalline Compound A Tartrate (“Form I1”)

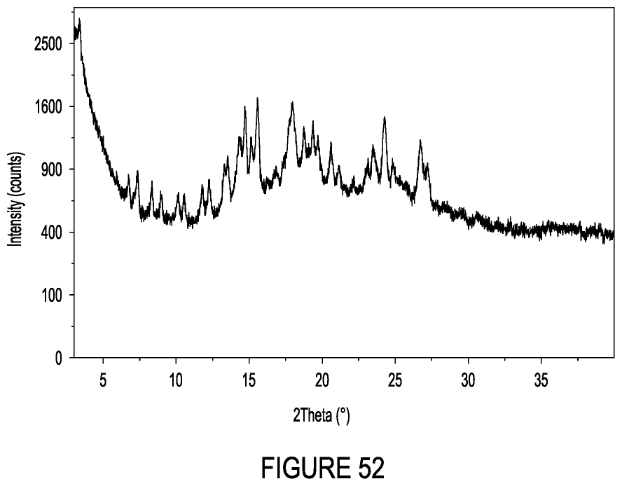

In some embodiments, the disclosure provides a crystalline Compound A tartrate salt (“Form I1”), characterized by an X-ray powder diffraction (XRPD) pattern comprising peaks at 3.4, 14.7, 15.6, 18.0, and 24.3±0.2° 2θ using CuKα radiation. In some embodiments, Form I1 is further characterized by XRPD pattern peaks at 14.3, 15.1, 18.7, 19.4, and 19.7±0.2° 2θ using CuKα radiation. In some embodiments, Form I1 is also characterized by XRPD pattern peaks at 13.3, 13.5, 20.6, 21.1, 23.1, 23.5, 24.9, 26.7, and 27.2±0.2° 2θ using CuKα radiation. In some embodiments, the disclosure provides crystalline Compound A tartrate salt Form I1 having an XRPD substantially as shown in FIG. 52.

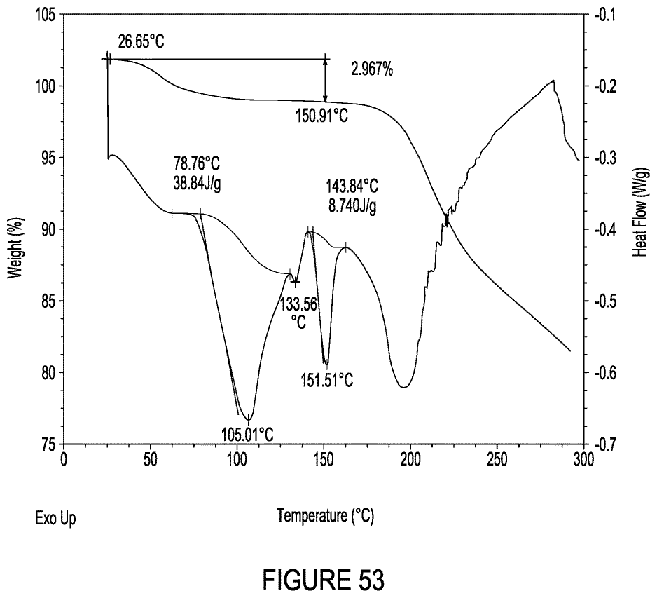

Form I1 can be characterized using DSC, TGA, and/or DVS. In some embodiments, the disclosure provides crystalline Compound A tartrate salt Form I1 having a DSC substantially as shown in FIG. 53.

In some embodiments, the disclosure provides crystalline Compound A tartrate salt Form I1 having a TGA substantially as shown in FIG. 53.

### Crystalline Compound A Ethanesulfonate (“Form J1”)

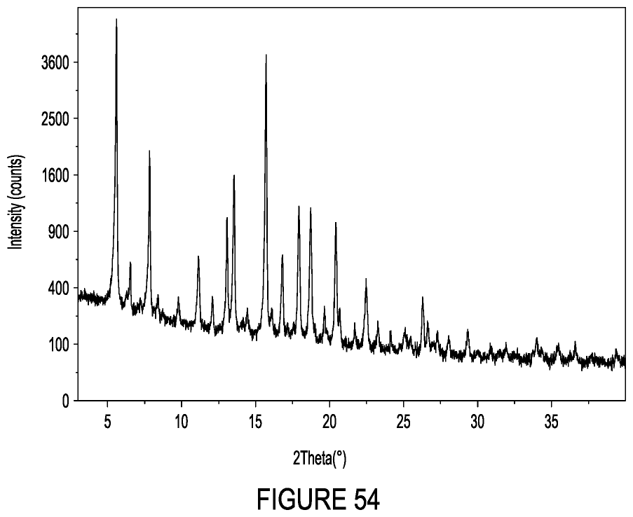

In some embodiments, the disclosure provides a crystalline Compound A ethanesulfonate salt (“Form J1”), characterized by an X-ray powder diffraction (XRPD) pattern comprising peaks at 5.6, 7.9, 13.6, 15.7, and 17.9±0.2° 2θ using CuKα radiation. In some embodiments, Form J1 is further characterized by XRPD pattern peaks at 6.6, 11.1, 13.1, 16.8, 18.7, and 20.4±0.2° 2θ using CuKα radiation. In some embodiments, the disclosure provides crystalline form of Compound A ethanesulfonate salt Form J1 having an XRPD substantially as shown in FIG. 54.

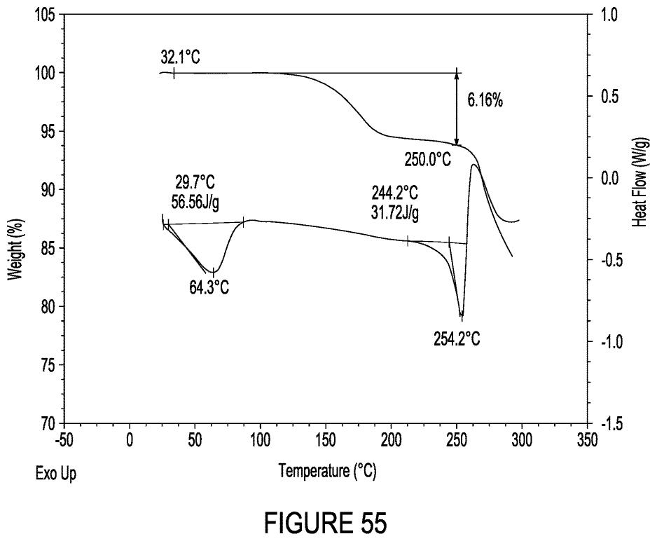

Form J1 can be characterized using DSC, TGA, and/or DVS. In some embodiments, the disclosure provides crystalline Compound A ethanesulfonate salt Form J1 having a DSC substantially as shown in FIG. 55.

In some embodiments, the disclosure provides crystalline Compound A ethanesulfonate salt Form J1 having a TGA substantially as shown in FIG. 55.

### Crystalline Compound A Ethanesulfonate (“Form J2”)

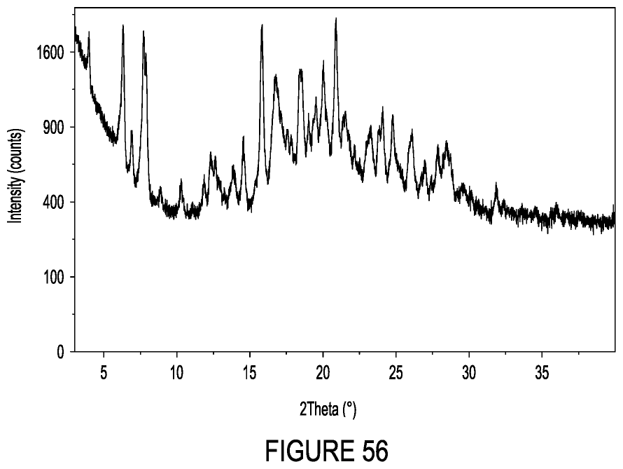

In some embodiments, the disclosure provides a crystalline Compound A ethanesulfonate salt (“Form J2”), characterized by an X-ray powder diffraction (XRPD) pattern comprising peaks at 4.0, 6.3, 7.7, 15.8, and 20.9±0.2° 2θ using CuKα radiation. In some embodiments, Form J2 is further characterized by XRPD pattern peaks at 7.9, 16.8, 18.4, 18.6, 19.5, and 20.0±0.2° 2θ using CuKα radiation. In some embodiments, Form J2 is also characterized by XRPD pattern peaks at 6.9, 17.6, 21.6, 23.3, 23.8, 24.1, 24.7, and 26.1±0.2° 2θ using CuKα radiation. In some embodiments, the disclosure provides crystalline form of Compound A ethanesulfonate salt Form J2 having an XRPD substantially as shown in FIG. 56.

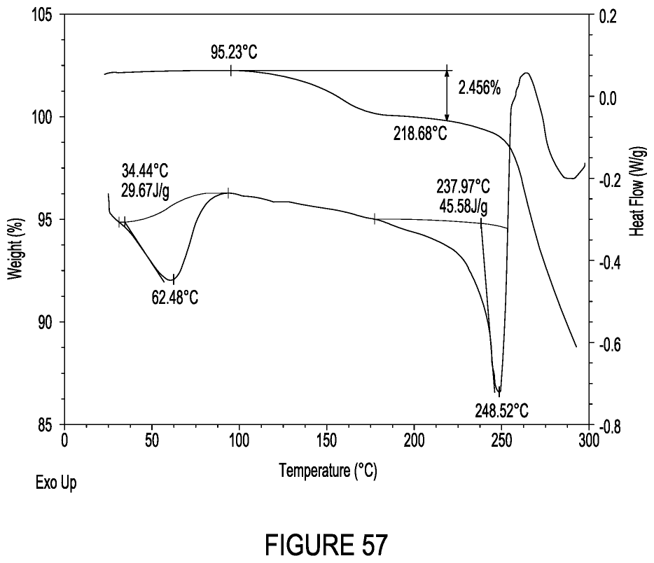

Form J2 can be characterized using DSC, TGA, and/or DVS. In some embodiments, the disclosure provides crystalline Compound A ethanesulfonate salt Form J2 having a DSC substantially as shown in FIG. 57.

In some embodiments, the disclosure provides crystalline Compound A ethanesulfonate salt Form J2 having a TGA substantially as shown in FIG. 57.

### Crystalline Compound A N-cyclohexylsulfamate (“Form K1”)

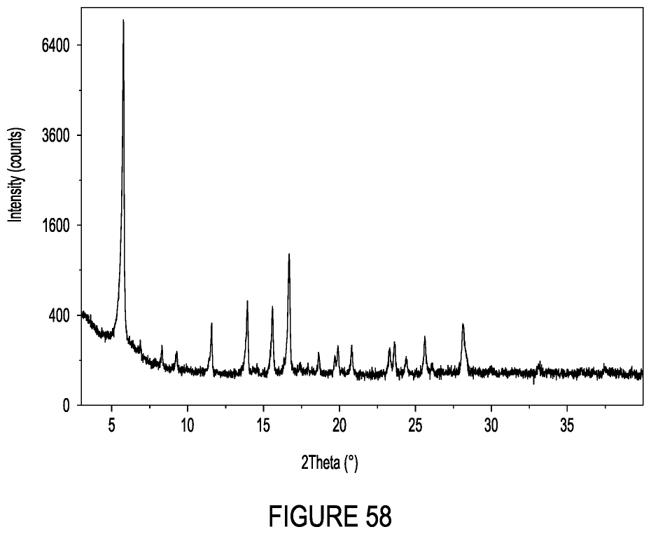

In some embodiments, the disclosure provides a crystalline Compound A N-cyclohexylsulfamate salt (“Form K1”), characterized by an X-ray powder diffraction (XRPD) pattern comprising peaks at 5.8, 14.0, 15.6, 16.7, and 28.1±0.2° 2θ using CuKα radiation. In some embodiments, the disclosure provides crystalline Compound A N-cyclohexylsulfamate salt Form K1 having an XRPD substantially as shown in FIG. 58.

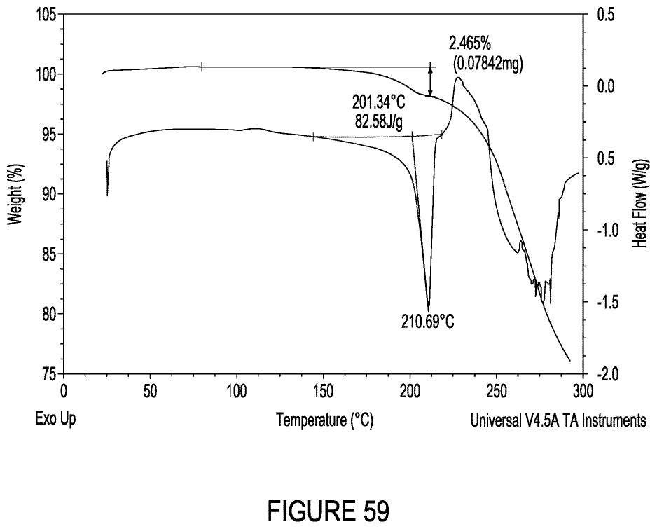

Form K1 can be characterized using DSC, TGA, and/or DVS. In some embodiments, the disclosure provides Form K1 having a DSC substantially as shown in FIG. 59.

In some embodiments, the disclosure provides Form K1 having a TGA substantially as shown in FIG. 59.

### Crystalline Compound A Maleate (“Form L1”)

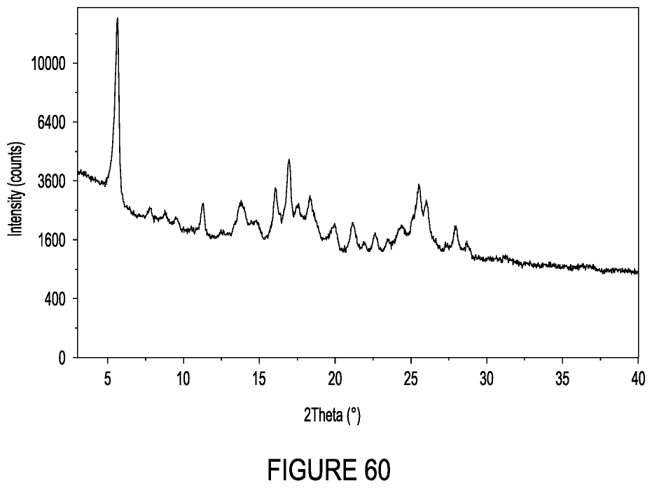

In some embodiments, the disclosure provides a crystalline Compound A maleate salt (“Form L1”), characterized by an X-ray powder diffraction (XRPD) pattern comprising peaks at 5.7, 17.0, 17.5, 25.6, and 26.1±0.2° 2θ using CuKα radiation. In some embodiments, the disclosure provides crystalline Compound A maleate salt Form L1 having an XRPD substantially as shown in FIG. 60.

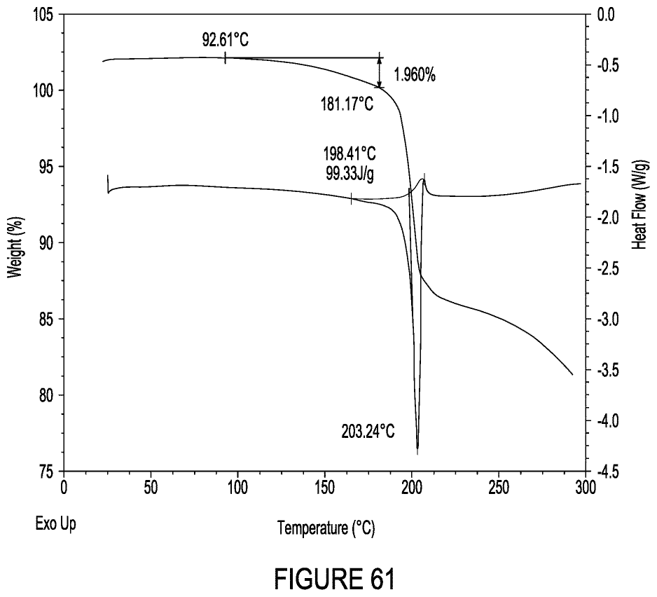

Form L1 can be characterized using DSC, TGA, and/or DVS. In some embodiments, the disclosure provides crystalline Compound A maleate salt Form L1 having a DSC substantially as shown in FIG. 61.

In some embodiments, the disclosure provides crystalline Compound A maleate salt Form L1 having a TGA substantially as shown in FIG. 61.

### Crystalline Compound A Phosphate (“Form M1”)

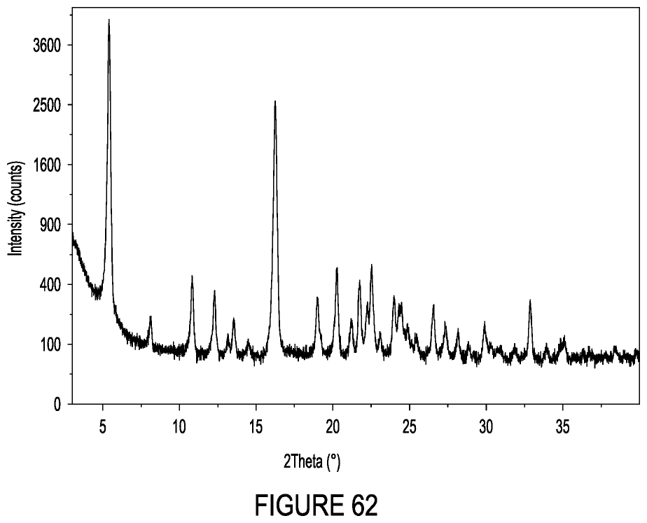

In some embodiments, the disclosure provides a crystalline Compound A phosphate salt (“Form M1”), characterized by an X-ray powder diffraction (XRPD) pattern comprising peaks at 5.4, 16.2, 20.3, and 22.5±0.2° 2θ using CuKα radiation. In some embodiments, Form M1 is further characterized by XRPD pattern peaks at 10.8, 12.3, 21.8, and 32.9±0.2° 2θ using CuKα radiation. In some embodiments, the disclosure provides crystalline Form M1 of Compound A phosphate salt having an XRPD substantially as shown in FIG. 62.

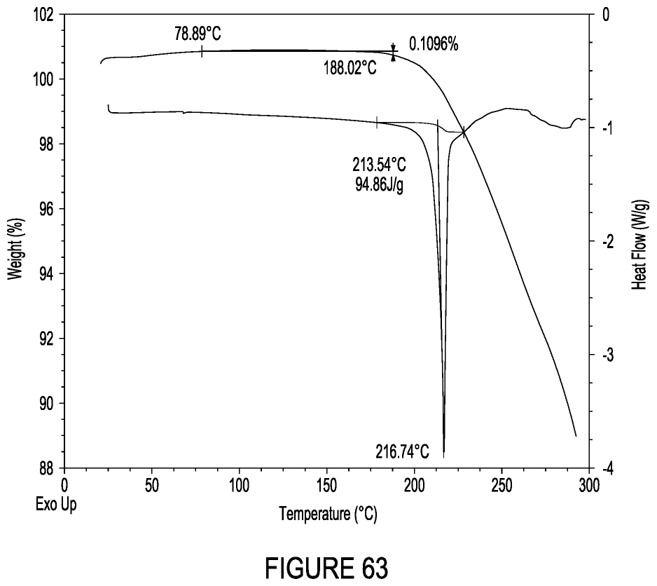

Form M1 can be characterized using DSC, TGA, and/or DVS. In some embodiments, the disclosure provides Form M1 having a DSC substantially as shown in FIG. 63.

In some embodiments, the disclosure provides Form M1 having a TGA substantially as shown in FIG. 63.

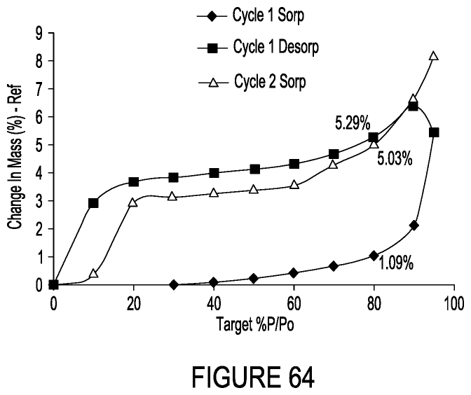

In some embodiments, the disclosure provides Form M1 having a DVS substantially as shown in FIG. 64.

### Crystalline Compound A Phosphate (“Form M2”)

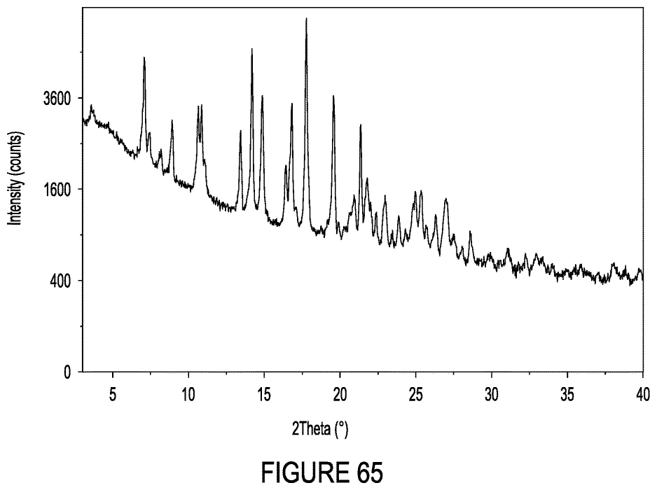

In some embodiments, the disclosure provides crystalline Compound A phosphate salt (“Form M2”), characterized by an X-ray powder diffraction (XRPD) pattern comprising peaks at 7.1, 14.2, 14.9, 17.8, and 19.6±0.2° 2θ using CuKα radiation. In some embodiments, Form M2 is further characterized by XRPD pattern peaks at 8.9, 10.6, 10.9, 13.4, 16.4, 16.8, and 21.4±0.2° 2θ using CuKα radiation. In some embodiments, Form M2 is also characterized by XRPD pattern peaks at 3.6, 7.4, 21.0, 21.8, 23.0, 25.0, 25.4, 26.3, and 26.9±0.2° 2θ using CuKα radiation. In some embodiments, the disclosure provides crystalline Form M2 of Compound A phosphate salt having an XRPD substantially as shown in FIG. 65.

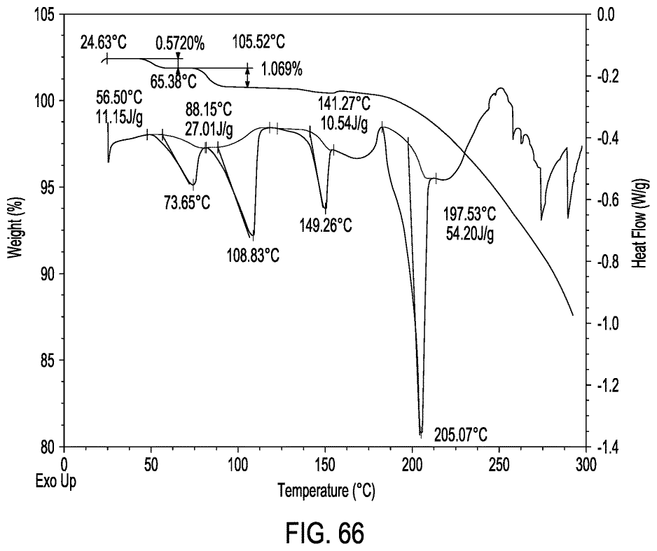

Form M2 can be characterized using DSC, TGA, and/or DVS. In some embodiments, the disclosure provides Form M2 having a DSC substantially as shown in FIG. 66.

In some embodiments, the disclosure provides Form M2 having a TGA substantially as shown in FIG. 66.

### Crystalline Compound A Phosphate (“Form M3”)

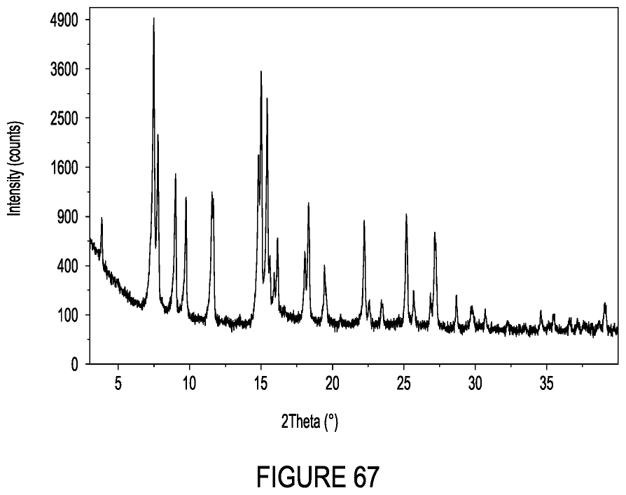

In some embodiments, the disclosure provides crystalline Compound A phosphate salt (“Form M3”), characterized by an X-ray powder diffraction (XRPD) pattern comprising peaks at 7.5, 7.8, 14.8, 15.0, and 15.4±0.2° 2θ using CuKα radiation. In some embodiments, Form M3 is further characterized by XRPD pattern peaks at 9.0, 9.8, 11.6, 11.7, 18.3, 22.2, and 25.2±0.2° 2θ using CuKα radiation. In some embodiments, Form M3 is also characterized by XRPD pattern peaks at 3.9, 16.2, 27.2, and 27.2±0.2° 2θ using CuKα radiation. In some embodiments, the disclosure provides crystalline Form M3 of Compound A phosphate salt having an XRPD substantially as shown in FIG. 67.

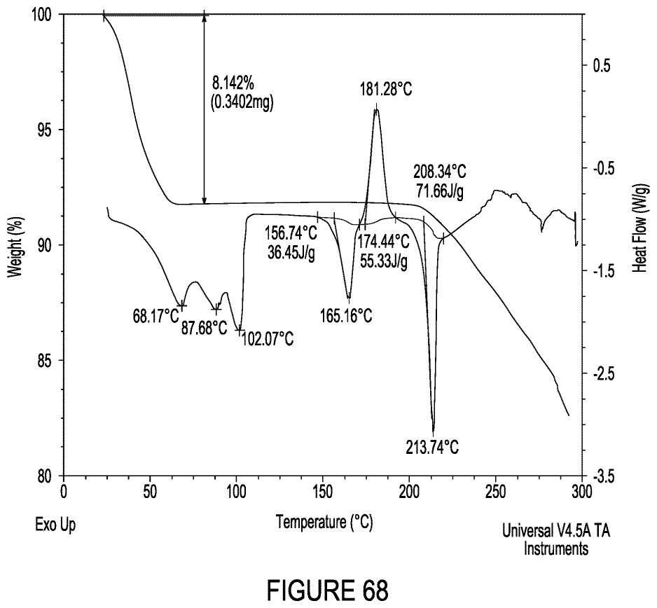

Form M3 can be characterized using DSC, TGA, and/or DVS. In some embodiments, the disclosure provides Form M3 having a DSC substantially as shown in FIG. 68.

In some embodiments, the disclosure provides Form M3 having a TGA substantially as shown in FIG. 68.

### Cocrystals

In some embodiments, the disclosure provides crystalline cocrystals comprising Compound A and a coformer. Nonlimiting examples of molecules suitable as coformers include, for example, salicylic acid, formic acid, benzoic acid, isobutyric acid, caprylic acid, sorbic acid, succinic acid, and adipic acid.

### Crystalline Compound A Salicylic Acid Cocrystal (“Form CC-1A”)

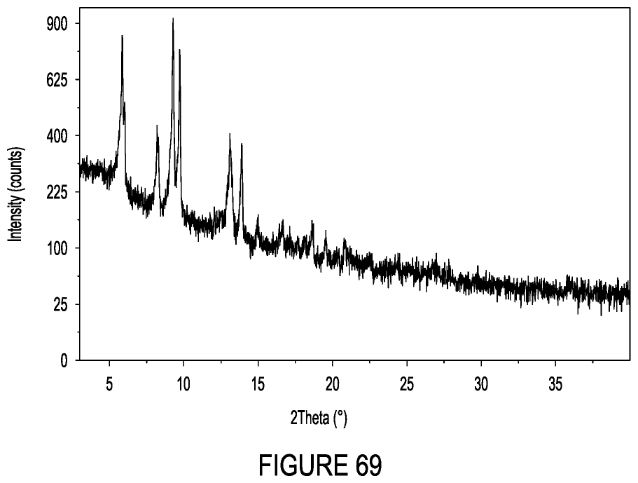

In some embodiments, the disclosure provides a crystalline Compound A salicylic acid cocrystal (“Form CC-1A”), characterized by an X-ray powder diffraction (XRPD) pattern comprising peaks at 9.3, 5.9, 9.7. 6.0, and 13.9±0.2° 2θ using CuKα radiation. In some embodiments, Form CC-1A is further characterized by XRPD pattern peaks at 13.1, 8.3, and 18.6±0.2° 2θ using CuKα radiation. In some embodiments, the disclosure provides crystalline Form CC-1A of Compound A salicylic acid cocrystal having an XRPD substantially as shown in FIG. 69.

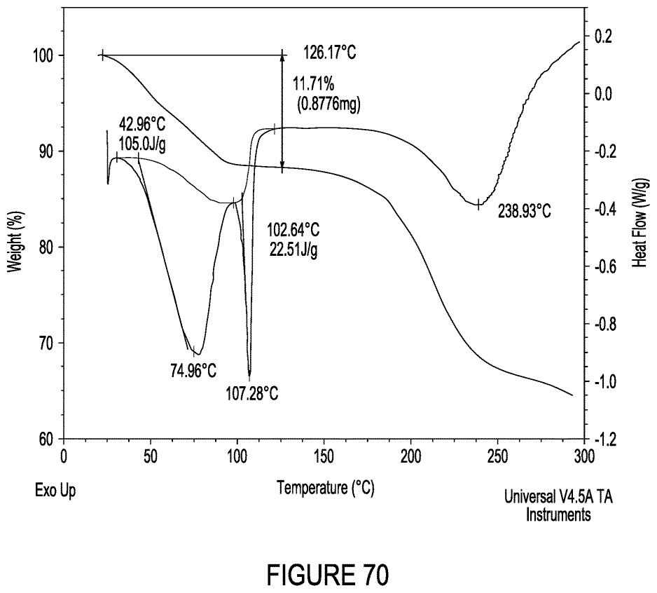

Form CC-1A can be characterized using DSC, TGA, and/or DVS. In some embodiments, the disclosure provides Form CC-1A having a DSC substantially as shown in FIG. 70.

In some embodiments, the disclosure provides Form CC-1A having a TGA substantially as shown in FIG. 70.

### Crystalline Compound A Salicylic Acid Cocrystal (“Form CC-2A”)

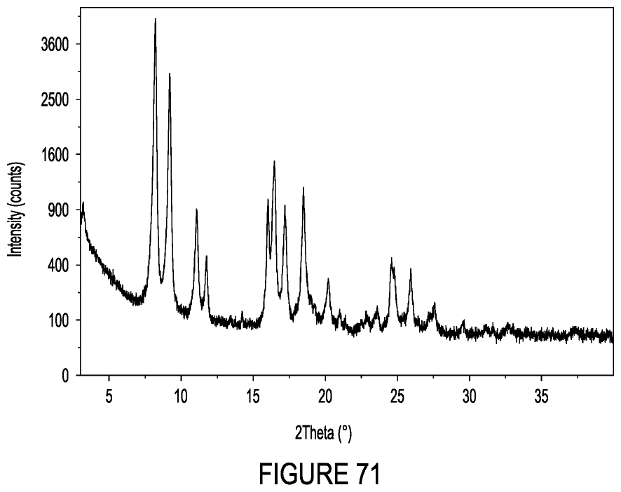

In some embodiments, the disclosure provides a crystalline Compound A salicylic acid cocrystal (“Form CC-2A”), characterized by an X-ray powder diffraction (XRPD) pattern comprising peaks at 8.2, 9.2, 16.5, 18.5, and 16.0±0.2° 2θ using CuKα radiation. In some embodiments, Form CC-2A is further characterized by XRPD pattern peaks at 17.2, 11.1, 3.2, 11.8, 24.6, and 25.9±0.2° 2θ using CuKα radiation. In some embodiments, the disclosure provides crystalline Form CC-2A of Compound A salicylic acid cocrystal having an XRPD substantially as shown in FIG. 71.

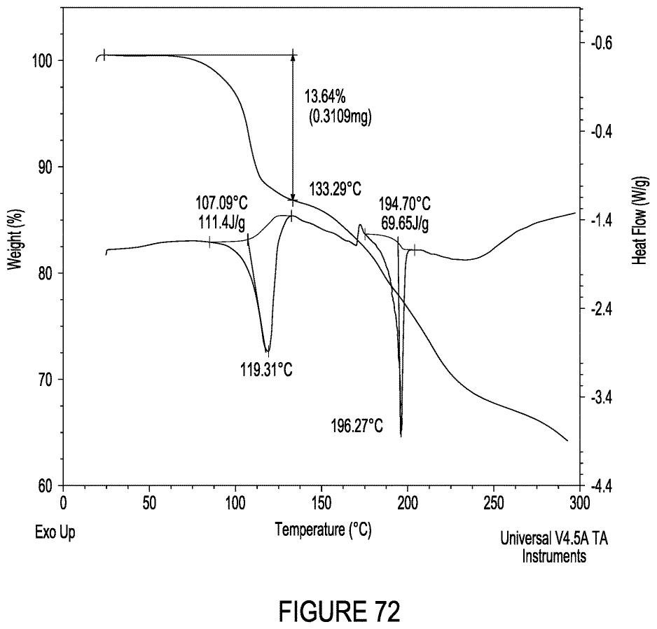

Form CC-2A can be characterized using DSC, TGA, and/or DVS. In some embodiments, the disclosure provides Form CC-2A having a DSC substantially as shown in FIG. 72.

In some embodiments, the disclosure provides Form CC-2A having a TGA substantially as shown in FIG. 72.

### Crystalline Compound A Salicylic Acid Cocrystal (“Form CC-3A”)

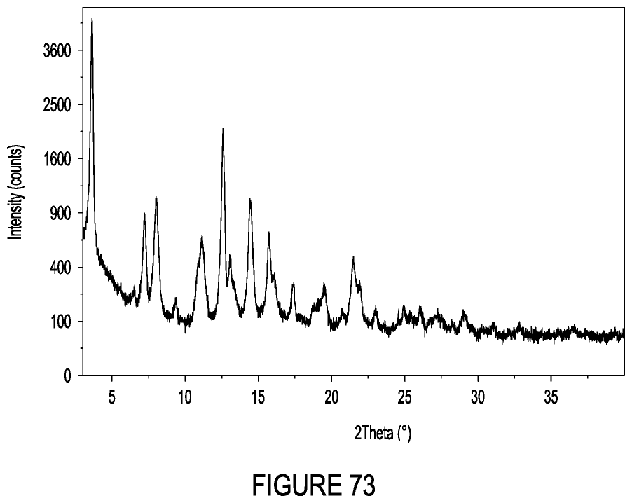

In some embodiments, the disclosure provides a crystalline Compound A salicylic acid cocrystal (“Form CC-3A”), characterized by an X-ray powder diffraction (XRPD) pattern comprising peaks at 3.6, 12.6, 8.0, 14.4, and 7.2±0.2° 2θ using CuKα radiation. In some embodiments, Form CC-3A is further characterized by XRPD pattern peaks at 15.7, 11.2, 13.0, 21.5, 10.8, and 16.1±0.2° 2θ using CuKα radiation. In some embodiments, the disclosure provides crystalline Form CC-3A of Compound A salicylic acid cocrystal having an XRPD substantially as shown in FIG. 73.

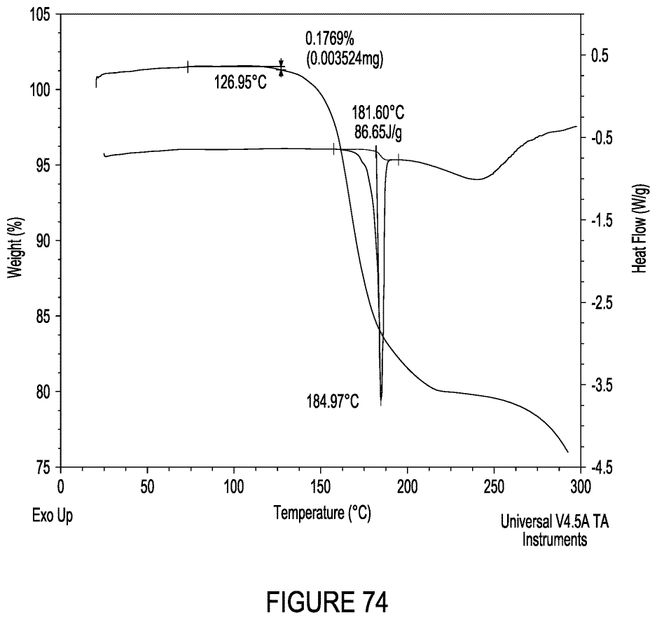

Form CC-3A can be characterized using DSC, TGA, and/or DVS. In some embodiments, the disclosure provides Form CC-3A having a DSC substantially as shown in FIG. 74.

In some embodiments, the disclosure provides Form CC-3A having a TGA substantially as shown in FIG. 74.

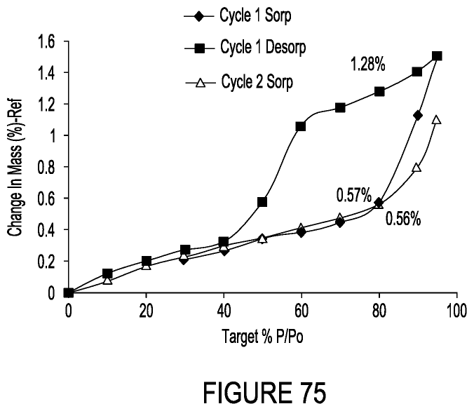

In some embodiments, the disclosure provides Form CC-3A having a DVS profile substantially as shown in FIG. 75.

### Crystalline Compound A Salicylic Acid Cocrystal (“Form CC-4A”)

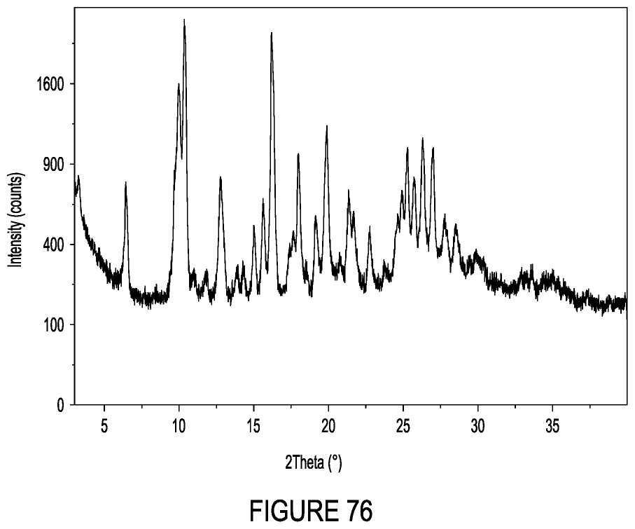

In some embodiments, the disclosure provides a crystalline Compound A salicylic acid cocrystal (“Form CC-4A”), characterized by an X-ray powder diffraction (XRPD) pattern comprising peaks at 10.3, 16.2, 9.9, 16.3, 19.9±0.2° 2θ using CuKα radiation. In some embodiments, Form CC-4A is further characterized by XRPD pattern peaks at 26.3, 27.0, 25.3, 18.0, 12.8, and 9.7±0.2° 2θ using CuKα radiation. In some embodiments, Form CC-4A is also characterized by XRPD pattern peaks at 28.5, 28.1, 24.8, 24.4, 23.4, and 22.3±0.2° 2θ using CuKα radiation. In some embodiments, Form CC-4A is also characterized by XRPD pattern peaks at 17.8, 16.2, 15.7, 15.67, 15.4, 13.6, and 13.4±0.2° 2θ using CuKα radiation. In some embodiments, the disclosure provides crystalline Form CC-4A of Compound A salicylic acid cocrystal having an XRPD substantially as shown in FIG. 76.

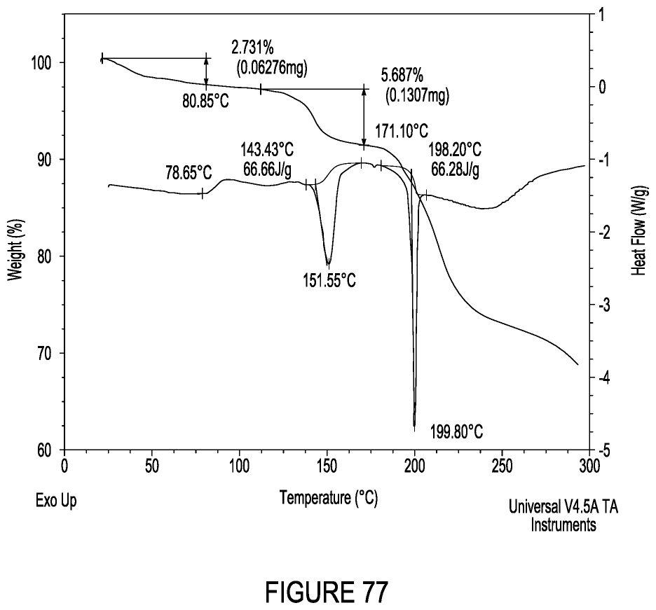

Form CC-4A can be characterized using DSC, TGA, and/or DVS. In some embodiments, the disclosure provides Form CC-4A having a DSC substantially as shown in FIG. 77.

In some embodiments, the disclosure provides Form CC-4A having a TGA substantially as shown in FIG. 77.

### Crystalline Compound A Salicylic Acid Cocrystal (“Form CC-5A”)

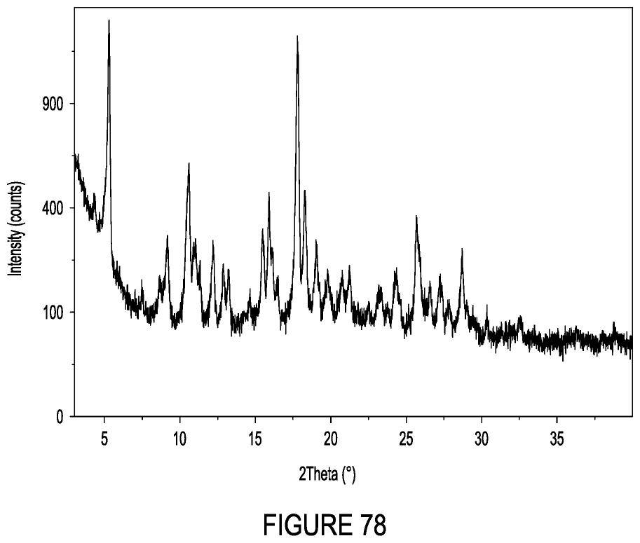

In some embodiments, the disclosure provides a crystalline Compound A salicylic acid cocrystal (“Form CC-5A”), characterized by an X-ray powder diffraction (XRPD) pattern comprising peaks at 5.3, 17.8, 10.6, 18.3, and 15.9±0.2° 2θ using CuKα radiation. In some embodiments, Form CC-5A is further characterized by XRPD pattern peaks at 25.7, 15.5, 19.1, 28.7, 9.2, 12.2, 11.0, and 12.9±0.2° 2θ using CuKα radiation. In some embodiments, Form CC-5A is also characterized by XRPD pattern peaks at 21.3, 19.8, 20.7, 24.3, 13.2, 26.6, 27.2, and 11.3±0.2° 2θ using CuKα radiation. In some embodiments, the disclosure provides crystalline Form CC-5A of Compound A salicylic acid cocrystal having an XRPD substantially as shown in FIG. 78.

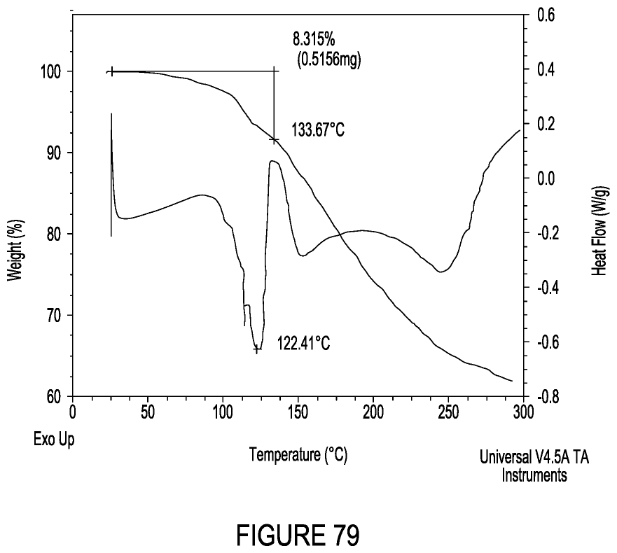

Form CC-5A can be characterized using DSC, TGA, and/or DVS. In some embodiments, the disclosure provides Form CC-5A having a DSC substantially as shown in FIG. 79.

In some embodiments, the disclosure provides Form CC-5A having a TGA substantially as shown in FIG. 79.

### Crystalline Compound A Formic Acid Cocrystal (“Form CC-1B”)

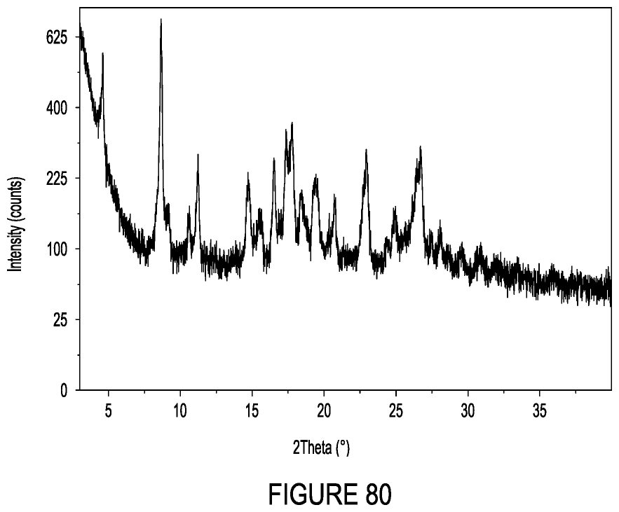

In some embodiments, the disclosure provides a crystalline Compound A formic acid cocrystal (“Form CC-1B”), characterized by an X-ray powder diffraction (XRPD) pattern comprising peaks at 8.6, 4.6, 17.8, 17.4, and 23.0±0.2° 2θ using CuKα radiation. In some embodiments, Form CC-1B is further characterized by XRPD pattern peaks at 11.3, 14.8, 15.7, 16.5, 18.4, 19.3, 20.7, 24.9, and 26.8±0.2° 2θ using CuKα radiation. In some embodiments, the disclosure provides crystalline Form CC-1B of Compound A formic acid cocrystal having an XRPD substantially as shown in FIG. 80.

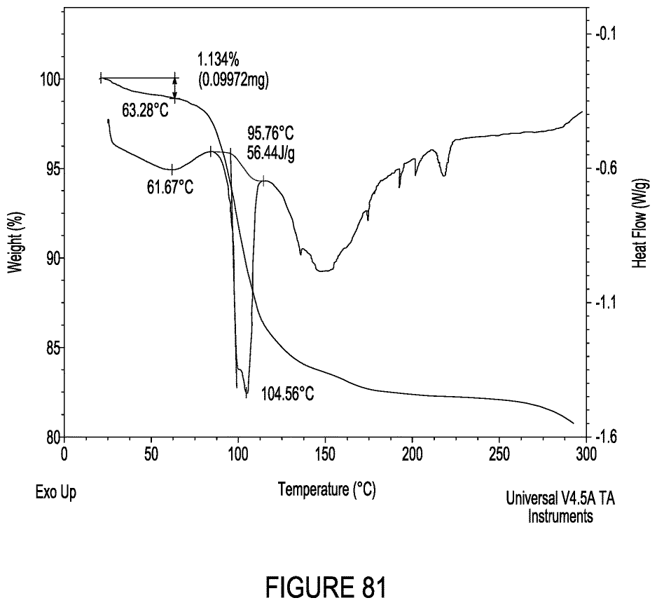

Form CC-1B can be characterized using DSC, TGA, and/or DVS. In some embodiments, the disclosure provides Form CC-1B having a DSC substantially as shown in FIG. 81.

In some embodiments, the disclosure provides Form CC-1B having a TGA substantially as shown in FIG. 81.

### Crystalline Compound A Benzoic Acid Cocrystal (“Form CC-1C”)

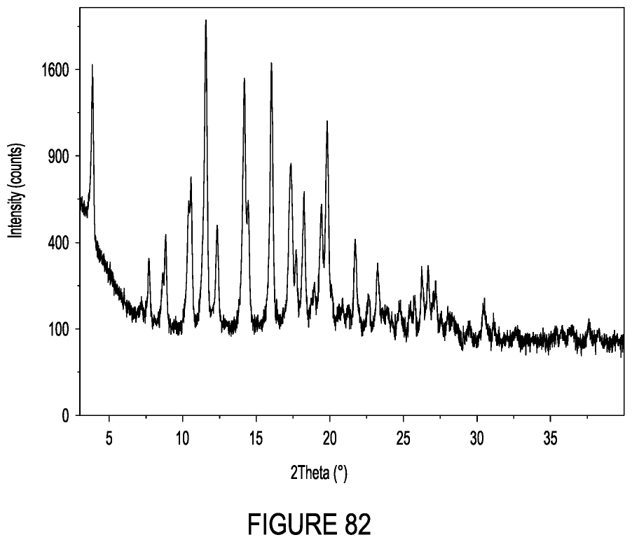

In some embodiments, the disclosure provides a crystalline Compound A benzoic acid cocrystal (“Form CC-1C”), characterized by an X-ray powder diffraction (XRPD) pattern comprising peaks at 11.6, 16.1, 14.2, 3.9, and 19.8±0.2° 2θ using CuKα radiation. In some embodiments, Form CC-1C is further characterized by XRPD pattern peaks at 10.4, 10.6, 12.4, 14.5, 17.3, 18.3, and 19.4±0.2° 2θ using CuKα radiation. In some embodiments, Form CC-1C is also characterized by XRPD pattern peaks at 7.7, 8.8, 17.7, 21.7, 23.2, 26.3, and 26.69±0.2° 2θ using CuKα radiation. In some embodiments, the disclosure provides crystalline Compound A benzoic acid cocrystal of Form CC-1C having an XRPD substantially as shown in FIG. 82.

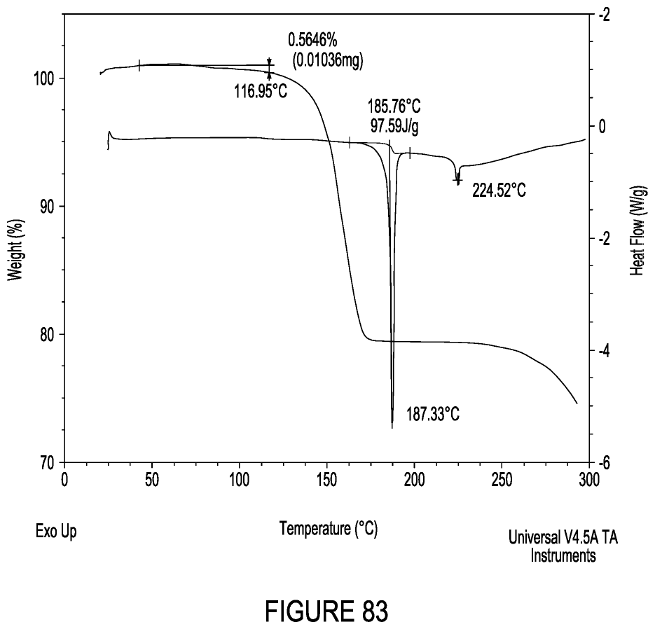

Form CC-1C can be characterized using DSC, TGA, and/or DVS. In some embodiments, the disclosure provides Form CC-1C having a DSC substantially as shown in FIG. 83.

In some embodiments, the disclosure provides Form CC-1C having a TGA substantially as shown in FIG. 83.

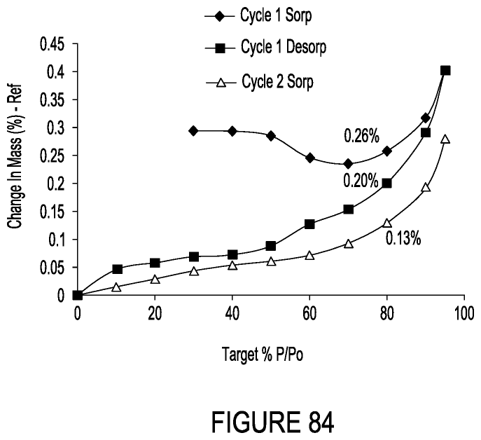

In some embodiments, the disclosure provides Form CC-1C having a DVS profile substantially as shown in FIG. 84.

### Crystalline Compound A Isobutyric Acid Cocrystal (“Form CC-1D”)

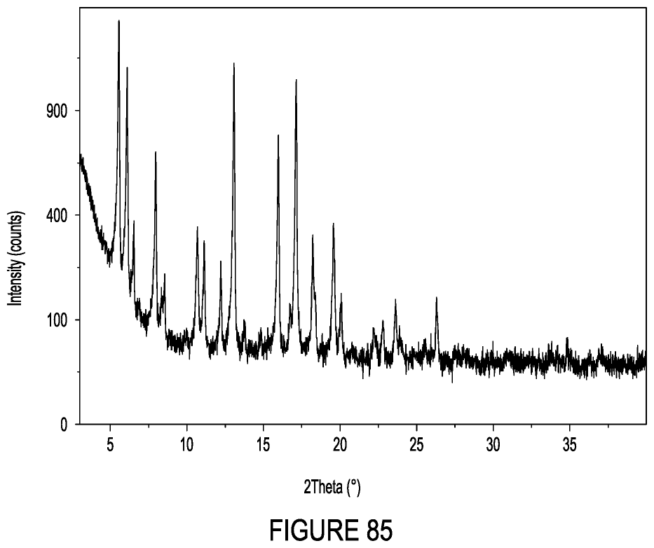

In some embodiments, the disclosure provides a crystalline Compound A isobutyric acid cocrystal (“Form CC-1D”), characterized by an X-ray powder diffraction (XRPD) pattern comprising peaks at 5.6, 6.1, 13.1, 16.0, and 17.1±0.2° 2θ using CuKα radiation. In some embodiments, Form CC-1D is further characterized by XRPD pattern peaks at 6.5, 8.0, 8.6, 10.7, 11.1, 12.2, and 19.6±0.2° 2θ using CuKα radiation. In some embodiments, the disclosure provides crystalline Compound A isobutyric acid cocrystal Form CC-1D having an XRPD substantially as shown in FIG. 85.

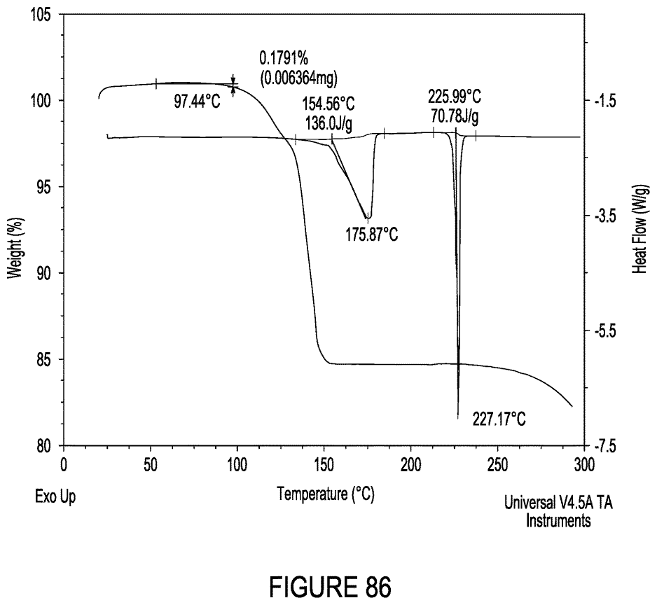

Form CC-1D can be characterized using DSC, TGA, and/or DVS. In some embodiments, the disclosure provides Form CC-1D having a DSC substantially as shown in FIG. 86.

In some embodiments, the disclosure provides Form CC-1D having a TGA substantially as shown in FIG. 86.

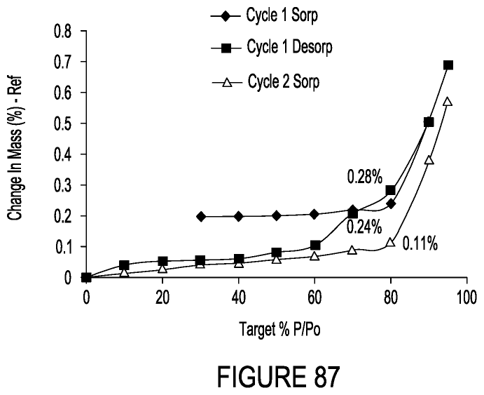

In some embodiments, the disclosure provides Form CC-1D having a DVS profile substantially as shown in FIG. 87.

### Crystalline Compound A Isobutyric Acid Cocrystal (“Form CC-2D”)

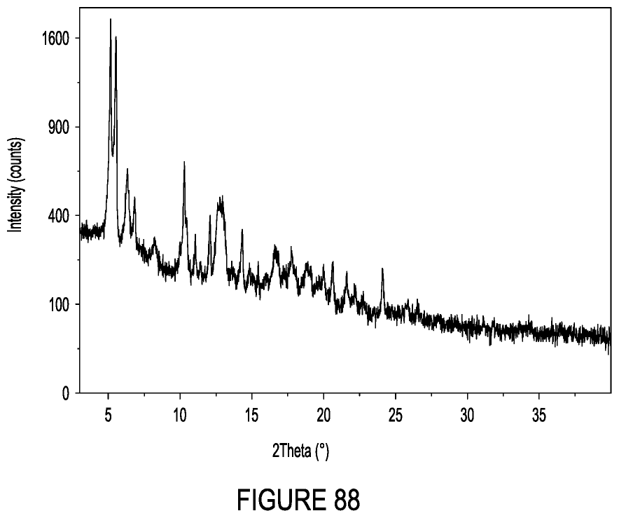

In some embodiments, the disclosure provides a crystalline Compound A isobutyric acid cocrystal (“Form CC-2D”), characterized by an X-ray powder diffraction (XRPD) pattern comprising peaks at 5.2, 5.5, 6.3, 10.3, and 12.6±0.2° 2θ using CuKα radiation. In some embodiments, Form CC-2D is further characterized by XRPD pattern peaks at 6.8, 12.1, 13.0, and 14.3±0.2° 2θ using CuKα radiation. In some embodiments, the disclosure provides crystalline Compound A isobutyric acid cocrystal Form CC-2D having an XRPD substantially as shown in FIG. 88.

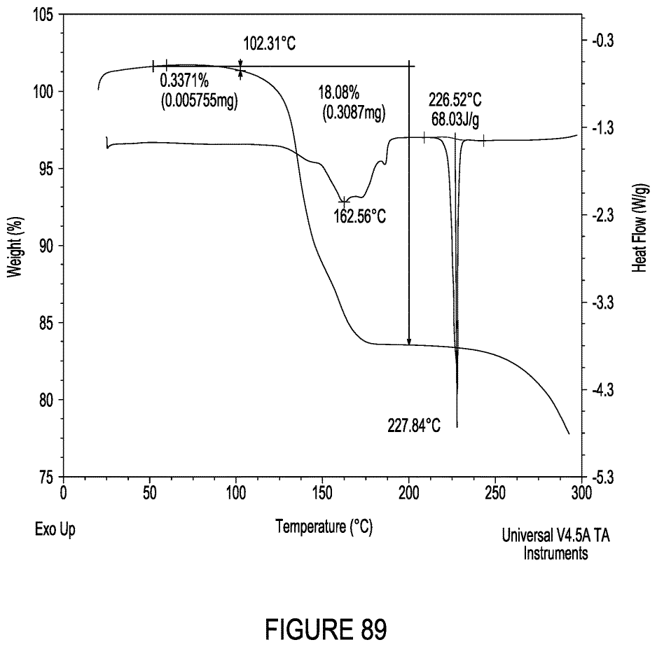

Form CC-2D can be characterized using DSC, TGA, and/or DVS. In some embodiments, the disclosure provides Form CC-2D having a DSC substantially as shown in FIG. 89.

In some embodiments, the disclosure provides Form CC-2D having a TGA substantially as shown in FIG. 89.

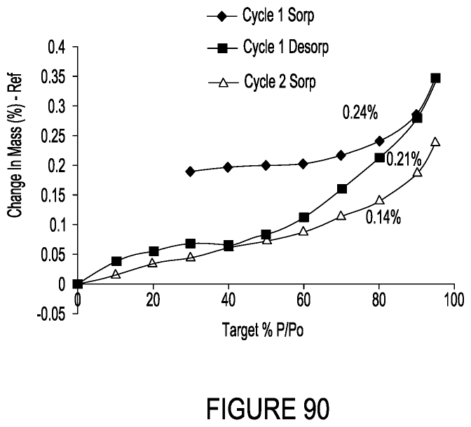

In some embodiments, the disclosure provides Form CC-2D having a DVS profile substantially as shown in FIG. 90.

### Crystalline Compound A Capyrlic Acid Cocrystal (“Form CC-1E”)

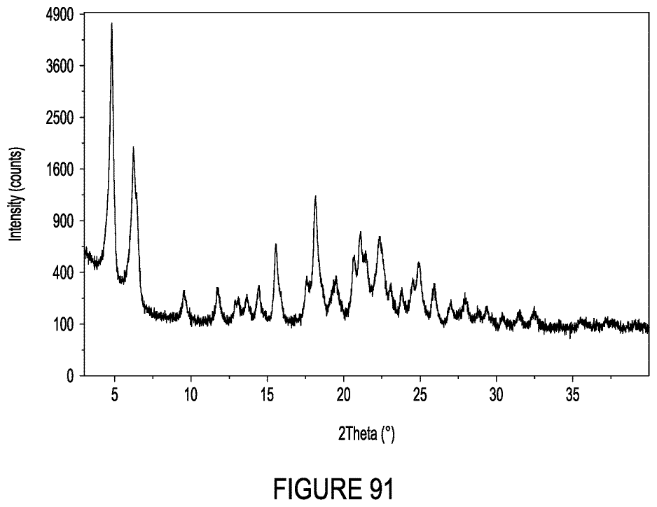

In some embodiments, the disclosure provides a crystalline Compound A caprylic acid cocrystal (“Form CC-1E”), characterized by an X-ray powder diffraction (XRPD) pattern comprising peaks at 4.8, 6.2, 6.5, 18.1, and 21.1±0.2° 2θ using CuKα radiation. In some embodiments, Form CC-1E is further characterized by XRPD pattern peaks at 15.6, 20.6, 21.4, 22.4, and 24.9±0.2° 2θ using CuKα radiation. In some embodiments, the disclosure provides crystalline Compound A caprylic acid cocrystal Form CC-1E having an XRPD substantially as shown in FIG. 91.

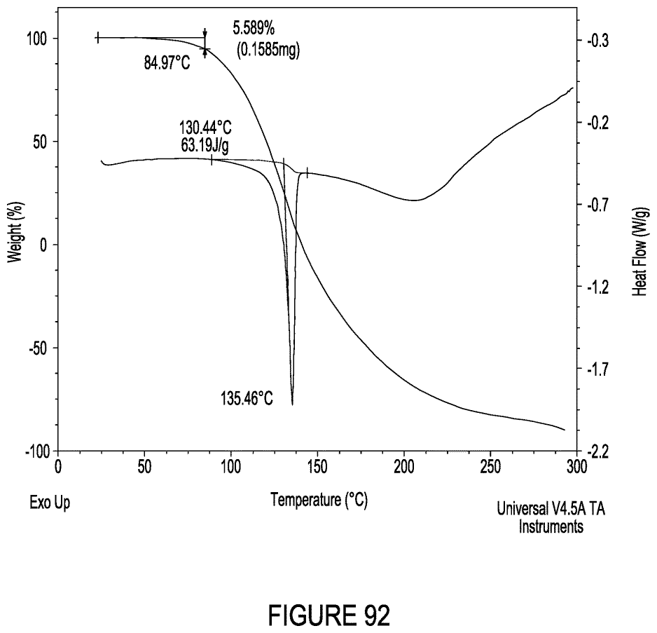

Form CC-1E can be characterized using DSC, TGA, and/or DVS. In some embodiments, the disclosure provides Form CC-1E having a DSC substantially as shown in FIG. 92.

In some embodiments, the disclosure provides Form CC-1E having a TGA substantially as shown in FIG. 92.

### Crystalline Compound A Sorbic Acid Cocrystal (“Form CC-1F”)

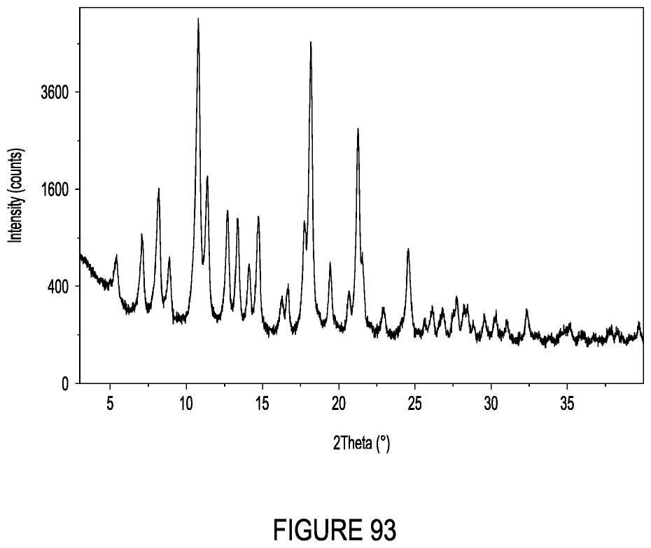

In some embodiments, the disclosure provides a crystalline Compound A sorbic acid cocrystal (“Form CC-1F”), characterized by an X-ray powder diffraction (XRPD) pattern comprising peaks at 8.2, 10.8, 11.4, 18.2, and 21.±0.2° 2θ using CuKα radiation. In some embodiments, Form CC-1F is further characterized by XRPD pattern peaks at 5.4, 7.1, 8.9, 12.7, 13.4, 14.8, 17.7, 21.6, and 24.6±0.2° 2θ using CuKα radiation. In some embodiments, the disclosure provides crystalline Compound A sorbic acid cocrystal Form CC-1F having an XRPD substantially as shown in FIG. 93.

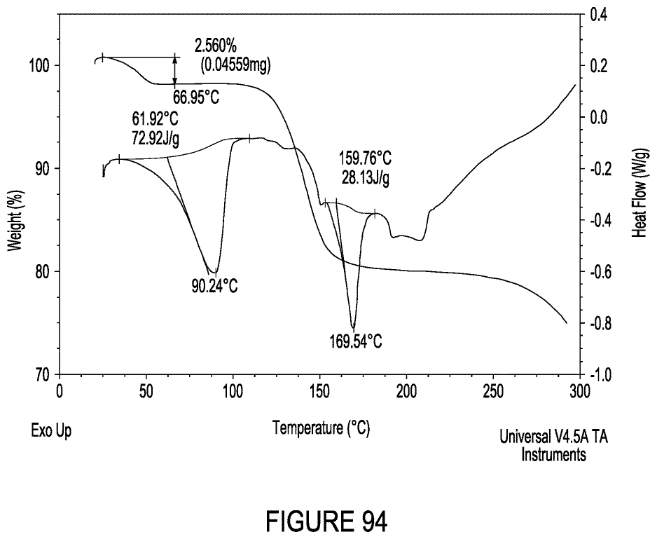

Form CC-1F can be characterized using DSC, TGA, and/or DVS. In some embodiments, the disclosure provides Form CC-1F having a DSC substantially as shown in FIG. 94.

In some embodiments, the disclosure provides Form CC-1F having a TGA substantially as shown in FIG. 94.

### Crystalline Compound A Saccharin Cocrystal (“Form CC-1G”)

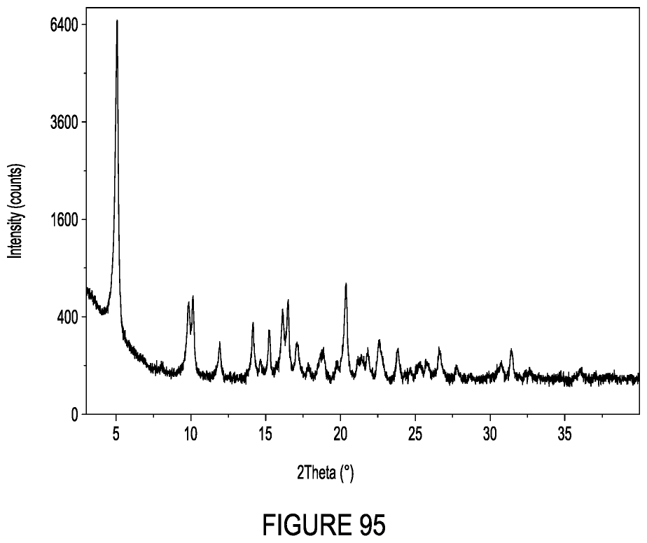

In some embodiments, the disclosure provides a crystalline Compound A saccharin cocrystal (“Form CC-1G”), characterized by an X-ray powder diffraction (XRPD) pattern comprising peaks at 5.1, 9.8, 10.1, 16.5, and 20.4. In some embodiments, the disclosure provides crystalline Compound A saccharin cocrystal Form CC-1G having an XRPD substantially as shown in FIG. 95.

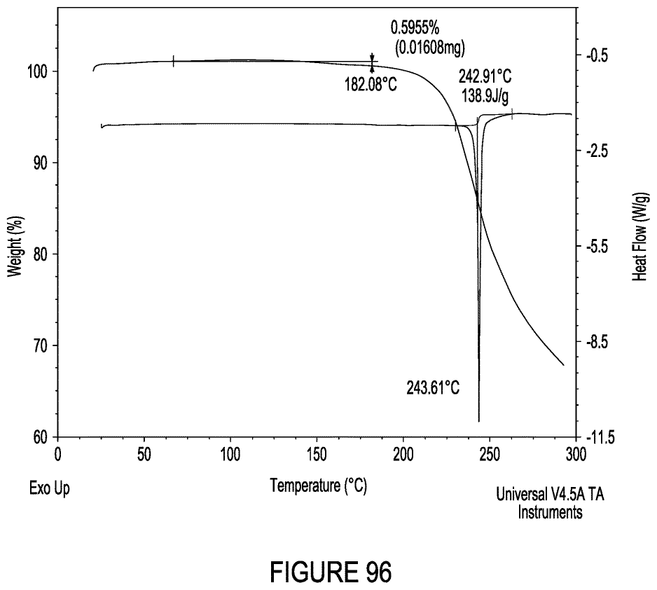

Form CC-1G can be characterized using DSC, TGA, and/or DVS. In some embodiments, the disclosure provides Form CC-1G having a DSC substantially as shown in FIG. 96.

In some embodiments, the disclosure provides Form CC-1G having a TGA substantially as shown in FIG. 96.

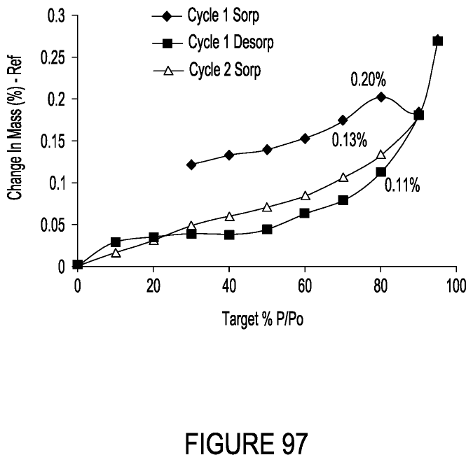

In some embodiments, the disclosure provides Form CC-1G having a DVS substantially as shown in FIG. 97.

### Crystalline Compound A Succinic Acid Cocrystal (“Form CC-1H”)

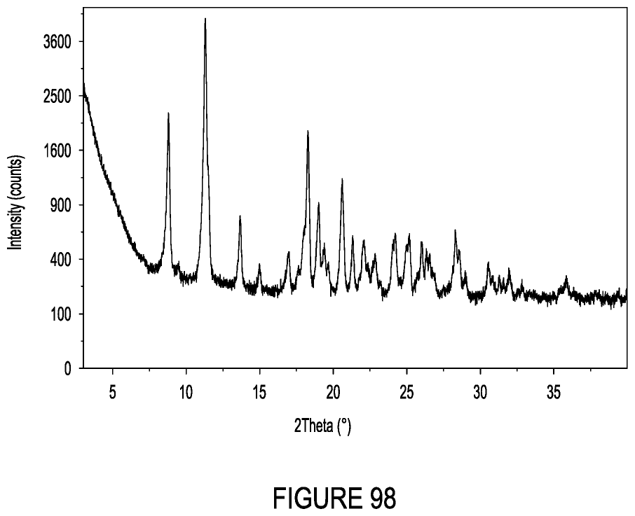

In some embodiments, the disclosure provides a crystalline Compound A succinic acid cocrystal (“Form CC-1H”), characterized by an X-ray powder diffraction (XRPD) pattern comprising peaks at 11.3, 11.5, 18.3, 19.0, and 20.6±0.2° 2θ using CuKα radiation. In some embodiments, Form CC-1H is further characterized by XRPD pattern peaks at 13.7, 24.2, 25.2, and 28.3±0.2° 2θ using CuKα radiation. In some embodiments, the disclosure provides crystalline Compound A succinic acid cocrystal Form CC-1H having an XRPD substantially as shown in FIG. 98.

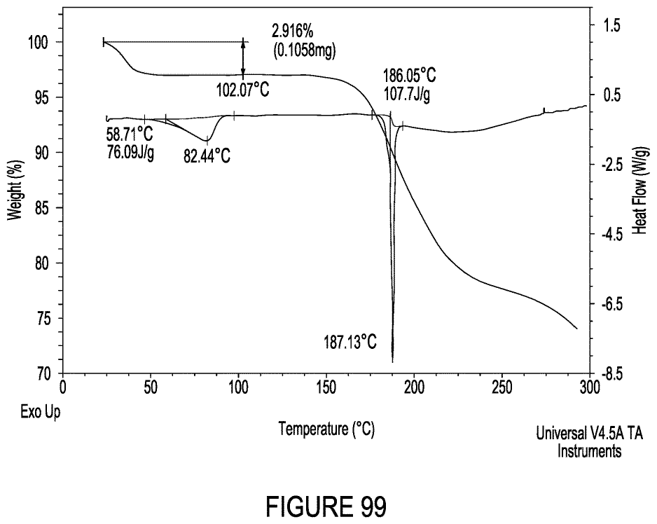

Form CC-1H can be characterized using DSC, TGA, and/or DVS. In some embodiments, the disclosure provides Form CC-1H having a DSC substantially as shown in FIG. 99.

In some embodiments, the disclosure provides Form CC-1H having a TGA substantially as shown in FIG. 99.

### Crystalline Compound A Succinic Acid Cocrystal (“Form CC-2H”)

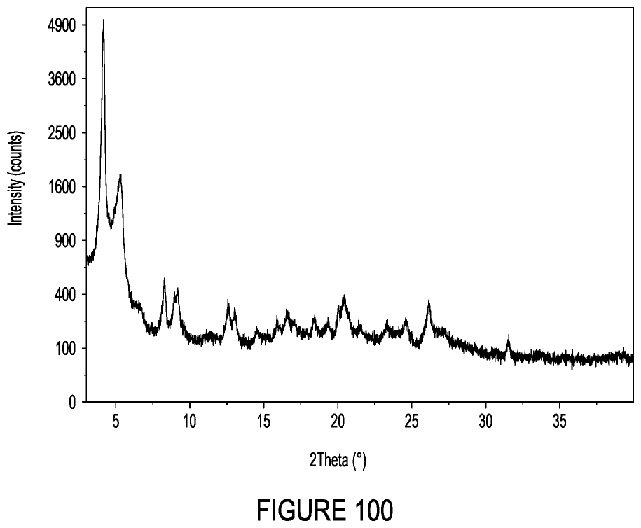

In some embodiments, the disclosure provides a crystalline Compound A succinic acid cocrystal (“Form CC-2H”), characterized by an X-ray powder diffraction (XRPD) pattern comprising peaks at 4.2, 5.3, 8.3, 9.0, and 9.2±0.2° 2θ using CuKα radiation. In some embodiments, the disclosure provides crystalline Compound A succinic acid cocrystal Form CC-2H having an XRPD substantially as shown in FIG. 100.

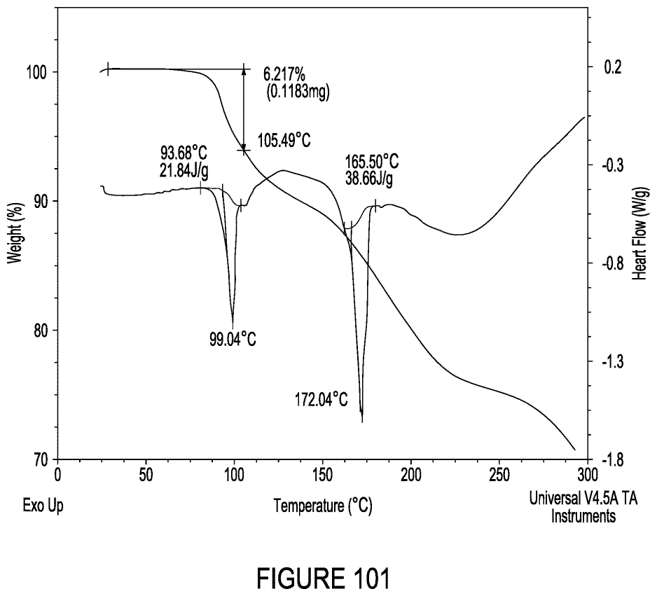

Form CC-2H can be characterized using DSC, TGA, and/or DVS. In some embodiments, the disclosure provides Form CC-2H having a DSC substantially as shown in FIG. 101.

In some embodiments, the disclosure provides Form CC-2H having a TGA substantially as shown in FIG. 101.

### Crystalline Compound A Adipic Acid Cocrystal (“Form CC-1l”)

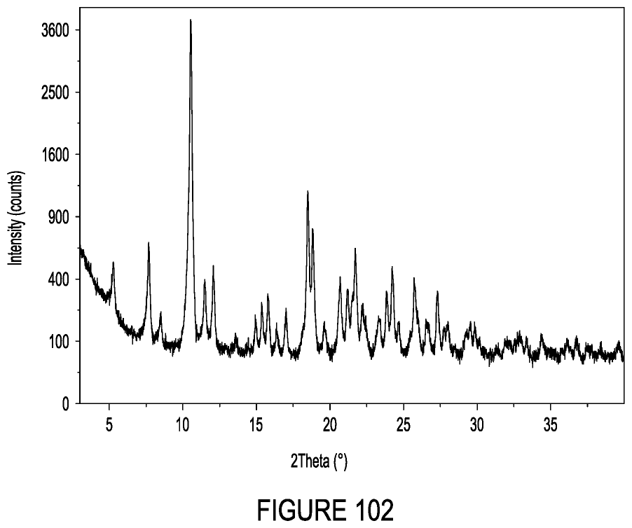

In some embodiments, the disclosure provides a crystalline Compound A adipic acid cocrystal (“Form CC-1I”), characterized by an X-ray powder diffraction (XRPD) pattern comprising peaks at 7.7, 10.5, 18.5, 18.9, and 21.7±0.2° 2θ using CuKα radiation. In some embodiments, Form CC-1I is further characterized by XRPD pattern peaks at 5.3, 12.1, 20.7, 24.2, and 25.7±0.2° 2θ using CuKα radiation. In some embodiments, the disclosure provides crystalline Compound A adipic acid cocrystal Form CC-1I having an XRPD substantially as shown in FIG. 102.

Form CC-1I can be characterized using DSC, TGA, and/or DVS. In some embodiments, the disclosure provides Form CC-1I having a DSC substantially as shown in FIG. 103.

In some embodiments, the disclosure provides Form CC-1I having a TGA substantially as shown in FIG. 103.

### Pharmaceutical Compositions

Further provided herein are pharmaceutical compositions of solid forms of Compound A, and methods of treating a subject suffering from cancer, comprising administering to the subject a therapeutically effective amount of the disclosed solid forms of Compound A.

In some embodiments, the disclosure provides a pharmaceutical composition comprising a crystalline form or amorphous form disclosed herein or cocrystal, or a pharmaceutically acceptable salt thereofand at least one pharmaceutically acceptable excipient. In some embodiments, the disclosure provides pharmaceutical compositions comprising Form 1.

In some examples, the crystalline form or amorphous form disclosed herein, or a pharmaceutically acceptable salt thereof is present in the pharmaceutical composition in an amount effective for the treatment of PRMT5-dependent cancers. In some aspects, the pharmaceutical composition is formulated for oral delivery or administration whereas in other embodiments, the pharmaceutical composition is formulated for intravenous delivery or administration. In some embodiments, the pharmaceutical composition is formulated for oral administration once a day or QD, and in some such formulations is a tablet where the effective amount of the active ingredient ranges from 1 mg to 2000 mg (e.g., 1, 25, 50, 100, 200, 400, 500, 750, 800, 1000, 1200, 1500, or 2000 mg).

### Methods of Treating a Subject

Further provided herein are methods of treating a subject suffering from cancer, comprising administering to the subject in need thereof a therapeutically effective amount of a crystalline form or amorphous form of Compound A, or pharmaceutically acceptable salt thereof, as disclosed herein, optionally as a pharmaceutical composition as disclosed herein. In some embodiments, the disclosure provides methods of treating a subject suffering from cancer comprising administering to the subject in need thereof a therapeutically effective amount of crystalline Form 1.

In some embodiments, the cancer is ovarian, lung, lymphoid, glioblastoma, colon, melanoma, gastric, pancreatic, or bladder cancer. In some embodiments, the cancer is non-small cell lung cancer. In some embodiments, the cancer is pancreatic cancer.

A MTAP-null cancer refers to a cancer that lacks expression of the enzyme methylthioadenosine phosphorylase (MTAP). The MTAP gene, located at chromosomal locus 9p21 is frequently co-deleted with the CDKN2A and CDKN2B genes. Selective MTAP deficiency, refers to deficiency without co-deletion of the CDKN2 genes, due either to selective deletion of the MTAP locus or to methylation of the MTAP promoter. MTAP-null cancers include MTAP-deficiency in at least 1% of disease cells. Terms “MTAP-null”, “MTAP-deficient” and “MTAP-negative” are used interchangeably.

An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In a patient afflicted with a MTAP-deficiency-related disease, it is possible that some disease cells (e.g., cancer cells) can be MTAP-deficient while others are not. Similarly, some disease cells may be MTA-accumulating while others are not. Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor. Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

In some embodiments, the cancer is acute myeloid leukemia, cancer in adolescents, childhood adrenocortical carcinoma childhood, AIDS-related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some cases, the cancer is pancreatic cancer; esophageal cancer; melanoma; lung cancer; mixed mullerian cancer; ovarian cancer; or gallbladder cancer.

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the MTAP-null cancer is lung cancer, biliary tract cancer, head and neck squamous cell carcinoma, pancreatic adenocarcinoma, gallbladder cancer, or mesothelioma.

In some embodiments, the MTAP-null cancer is lung cancer. In some embodiments the lung cancer is non-squamous cell lung cancer (NSCLC).

In some embodiments, the MTAP-null cancer is a solid tumor. Exemplary MTAP-null solid tumors include, but are not limited to, MTAP-null brain cancer (including, but not limited to, MTAP-null glioma, MTAP-null oligodendroglioma, MTAP-null glioblastoma multiforme, MTAP-null astrocytoma, MTAP-null medulloblastoma, MTAP-null ependymoma, and MTAP-null meningioma), MTAP-null head and neck cancer (including, but not limited to, MTAP-null salivary gland (parotid) tumors, MTAP-null head and neck squamous cell carcinoma, and MTAP-null thyroid cancer), MTAP-null breast cancer (including, but not limited to, invasive ductal breast cancer, mixed mucinous breast cancer and lobular carcinoma), MTAP-null mesothelioma, MTAP-null gastrointestinal tract cancer (including but not limited to, MTAP-null esophageal cancer (including, but not limited to, adenocarcinoma and squamous cell carcinoma), MTAP-null gastro-esophageal junction cancer, MTAP-null stomach cancer (including, but not limited to, adenocarcinoma and signet ring cell carcinoma), MTAP-null small bowel cancer, MTAP-null colon cancer, MTAP-null rectal cancer and MTAP-null gastrointestinal stromal tumor), MTAP-null neuroendocrine tumor, MTAP-null hepatobiliary cancer (including, but not limited to, MTAP-null biliary tract cancer (including cholangiocarcinoma, gallbladder cancer and ampullary cancer) and MTAP-null hepatocellular carcinoma), MTAP-null pancreatic cancer (including pancreatic adenocarcinoma), MTAP-null kidney cancer (including, but not limited to, MTAP-null renal cell carcinoma), MTAP-null adrenocortical carcinoma, MTAP-null bladder cancer (including, but not limited to, MTAP-null urothelial carcinoma), MTAP-null adrenocortical carcinoma, MTAP-null endometrial cancer, MTAP-null uterine cancer, MTAP-null testicular cancer, MTAP-null germ cell tumor, or MTAP-null prostate cancer, MTAP-null sarcoma or MTAP-null bone cancer (including, but not limited to, MTAP-null osteosarcoma, MTAP-null chondrosarcoma, MTAP-null soft tissue sarcoma, MTAP-null Ewing sarcoma, MTAP-null liposarcoma, MTAP-null leiomyosarcoma, and MTAP-null myxofibrosarcoma), MTAP-null cutaneous tumors (MTAP-null cutaneous squamous cell carcinoma and MTAP-null melanoma), MTAP-null nerve sheath tumor and MTAP-null cancer of unknown primary (CUP).

In some embodiments, the MTAP-null cancer is a hematologic tumor. Exemplary hematologic tumors include, but are not limited to, MTAP-null leukemia (including, but not limited to, MTAP-null acute lymphocytic leukemia, MTAP-null acute myeloid leukemia), MTAP-null lymphoma (including, but not limited to, MTAP-null mantle cell lymphoma, MTAP-null follicular lymphoma, MTAP-null diffuse large B cell lymphoma, and MTAP-null mycosis fungoides).

In some embodiments, the cancer is not a primary brain tumor or lymphoma.

In some embodiments, the subject does not have, and has not had, interstitial lung disease or pneumonitis.

### EMBODIMENTS

1. A crystalline form of Compound A:

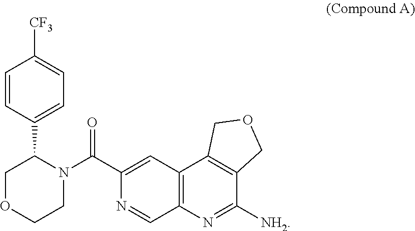 [SMILES: Nc1nc2cnc(C(=O)N3CCOC[C@@H]3c3ccc(C(F)(F)F)cc3)cc2c2c1COC2]

2. The crystalline form of embodiment 1, wherein Compound A is in free base from.

3. The crystalline form of embodiment 1 or 2, as Compound A free base (“Form 1”), characterized by an X-ray powder diffraction (XRPD) pattern comprising peaks at 4.5, 9.0, 13.3, 16.2, and 18.8±0.2° 2θ using CuKα radiation.

4. The crystalline form of embodiment 1 or 2, characterized by an X-ray powder diffraction (XRPD) pattern comprising at least three peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

5. The crystalline form of embodiment 1 or 2, characterized by an X-ray powder diffraction (XRPD) pattern comprising at least five peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

6. The crystalline form of embodiment 1 or 2, characterized by an X-ray powder diffraction (XRPD) pattern comprising at least seven peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

7. The crystalline form of embodiment 1 or 2, characterized by an X-ray powder diffraction (XRPD) pattern comprising at least eight peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

8. The crystalline form of any one of embodiments 1-7, characterized by a differential scanning calorimetery (DSC) thermograph comprising an endotherm with an onset of 225° C.±3° C.

9. The crystalline form of any one of embodiments 1-8, characterized by ¹³C solid state NMR comprising at least three peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

10. The crystalline form of any one of embodiments 1-8, characterized by ¹³C solid state NMR comprising at least five peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm

11. The crystalline form of any one of embodiments 1-8, characterized by ¹³C solid state NMR comprising at least seven peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

12. The crystalline form of any one of embodiments 1-11, characterized by ¹⁹F solid state NMR comprising peaks at −62.0 and −63.9 ppm.

13. A crystalline form of Compound A free base (“Form 3”), characterized by an X-ray powder diffraction (XRPD) pattern comprising peaks at 4.3, 12.6, 14.4, 16.2, and 25.6±0.2° 2θ using CuKα radiation.

14. The crystalline form of embodiment 13, further characterized by XRPD pattern peaks at 8.6, 13.9, 15.6, 16.7, and 25.3±0.2° 2θ using CuKα radiation.

15. The crystalline form of embodiment 13 or 14, further characterized by XRPD pattern peaks at 18.4, 19.7, 20.3, 26.7, 27.9, and 28.4±0.2° 2θ using CuKα radiation.

16. A crystalline form of Compound A free base (“Form 6”), characterized by an X-ray powder diffraction (XRPD) pattern comprising peaks at 4.5, 8.6, 9.0, 12.9, and 14.9±0.2° 2θ using CuKα radiation.

17. The crystalline form of embodiment 16, further characterized by XRPD pattern peaks at 7.4, 12.3, 13.3, 16.2, 17.8, and 18.8±0.2° 2θ using CuKα radiation.

18. A crystalline form of Compound A free base (“Form 7”), characterized by an X-ray powder diffraction (XRPD) pattern comprising peaks at 4.3, 8.5, 12.3, 13.1, and 14.8±0.2° 2θ using CuKα radiation.

19. The crystalline form of embodiment 18, further characterized by XRPD pattern peaks at 15.5, 19.1, and 20.9±0.2° 2θ using CuKα radiation.

20. A crystalline form of Compound A free base hydrate (“Form 8”), characterized by an X-ray powder diffraction (XRPD) pattern comprising peaks at 4.0, 7.7, 8.0, 12.3, and 15.1±0.2° 2θ using CuKα radiation.

21. The crystalline form of embodiment 20, further characterized by XRPD pattern peaks at 6.1, 10.0, 13.7, 17.0, 18.4, and 19.9±0.2° 2θ using CuKα radiation.

22. A crystalline form of Compound A ethanol solvate (“Form 2A”), characterized by an X-ray powder diffraction (XRPD) pattern comprising peaks at 5.8, 11.6, 12.7, 18.0, and 25.7±0.2° 2θ using CuKα radiation.

23. The crystalline form of embodiment 22, further characterized by XRPD pattern peaks at 16.5, 22.6, 23.3, and 25.8±0.2° 2θ using CuKα radiation.

24. A crystalline form of Compound A isopropanol solvate (“Form 3A”), characterized by an X-ray powder diffraction (XRPD) pattern comprising peaks at 5.6, 12.8, 16.4, 17.5, and 25.1±0.2° 2θ using CuKα radiation.

25. The crystalline form of embodiment 24, further characterized by XRPD pattern peaks at 8.2, 11.2, 12.5, 18.6, and 21.8±0.2° 2θ using CuKα radiation.

26. The crystalline form of embodiment 24 or 25, further characterized by XRPD pattern peaks at 6.4, 8.8, 9.2, 13.6, 21.0, 22.4, 22.9, 24.5, and 25.9±0.2° 2θ using CuKα radiation.

27. A crystalline form of Compound A acetone solvate (“Form 4A”), characterized by an X-ray powder diffraction (XRPD) pattern comprising peaks at 5.7, 7.7, 11.5, 14.8, and 15.3±0.2° 2θ using CuKα radiation.

28. The crystalline form of embodiment 27, further characterized by XRPD pattern peaks at 4.3, 7.3, 13.4, 16.2, and 24.0±0.2° 2θ using CuKα radiation.

29. The crystalline form of embodiment 27 or 28, further characterized by XRPD pattern peaks at 11.2, 18.5, 19.6, and 20.4±0.2° 2θ using CuKα radiation.

30. A crystalline form of Compound A methanol solvate (“Form 5A”), characterized by an X-ray powder diffraction (XRPD) pattern comprising peaks at 4.8, 7.7, 12.3, 15.3, and 16.1±0.2° 2θ using CuKα radiation.

31. The crystalline form of embodiment 30, further characterized by XRPD pattern peaks at 8.9, 10.5, 13.1, 13.4, 14.5, 17.6, 21.5, and 25.0±0.2° 2θ using CuKα radiation.

32. The crystalline form of embodiment 30 or 31, further characterized by XRPD pattern peaks at 9.7, 14.1, 18.7, 19.6, 22.7, 24.7, 26.0, and 26.6±0.2° 2θ using CuKα radiation.

33. A crystalline form of Compound A methyl tetrahydrofuran solvate (“Form 6A”) characterized by a XRPD pattern comprising peaks at 7.6, 11.3, 15.1, 18.3, and 28.0±0.2° 2θ using CuKα radiation.

34. The crystalline form of embodiment 33, further characterized by XRPD pattern peaks at 12.4, 15.7, 17.3, 17.7, 18.8, 19.7, 21.3, 22.7, 25.8, 26.1, and 26.5±0.2° 2θ using CuKα radiation.

35. The crystalline form of embodiment 33 or 34, further characterized by XRPD pattern peaks at 13.6, 14.2, 16.5, 16.9, 20.5, 26.9, and 28.4±0.2° 2θ using CuKα radiation.

36. A crystalline form of Compound A toluene sulfonate salt (“Form A1”), characterized by an X-ray powder diffraction (XRPD) pattern comprising peaks at 6.0, 19.0, 20.3, 24.1, 24.9, and 28.9±0.2° 2θ using CuKα radiation.

37. The crystalline form of embodiment 36, further characterized by XRPD pattern peaks at 9.5, 11.9, 14.3, 17.9, 26.2, 33.8, and 35.4±0.2° 2θ using CuKα radiation.

38. A crystalline form of Compound A toluene sulfonate salt (“Form A2”), characterized by an X-ray powder diffraction (XRPD) pattern comprising peaks at 12.7, 15.5, 16.2, 18.7, 19.7, and 21.8±0.2° 2θ using Cu Kα radiation.

39. The crystalline form of embodiment 38, further characterized by XRPD pattern peaks at 10.2, 23.2, 24.1, 24.6, 26.3, and 27.2±0.2° 2θ using CuKα radiation.

40. A crystalline form of Compound A toluene sulfonate salt (“Form A3”), characterized by an X-ray powder diffraction (XRPD) pattern comprising peaks at 4.9, 15.2, 18.8, 19.5, and 24.5±0.2° 2θ using Cu Kα radiation.

41. A crystalline form of Compound A benzene sulfonate salt (“Form B1”), characterized by an X-ray powder diffraction (XRPD) pattern comprising peaks at 5.1, 16.1, 17.9, 19.0, and 25.2±0.2° 2θ using CuKα radiation.

42. The crystalline form of embodiment 41, further characterized by XRPD pattern peaks at 20.2, 20.6, 23.2, 26.0, 27.0, and 30.4±0.2° 2θ using CuKα radiation.

43. A crystalline form of Compound A chloride salt (“Form C1”), characterized by an X-ray powder diffraction (XRPD) pattern comprising peaks at 6.8, 11.9, 16.6, 20.7, 23.8, 25.3, and 27.6±0.2° 2θ using Cu Kα radiation.

44. A crystalline form of Compound A chloride salt (“Form C2”), characterized by an X-ray powder diffraction (XRPD) pattern comprising peaks at 4.2, 5.6, 12.2, 12.9, and 18.1±0.2° 2θ using Cu Kα radiation.

45. The crystalline form of embodiment 44, further characterized by XRPD pattern peaks at 6.8, 7.1, 9.4, 13.4, 14.0, 16.5, and 17.7±0.2° 2θ using CuKα radiation.

46. The crystalline form of embodiment 44 or 45, further characterized by XRPD pattern peaks at 7.8, 8.7, 10.9, 11.8, 14.9, 15.4, and 19.1±0.2° 2θ using CuKα radiation.

47. A crystalline form of Compound A sulfate salt (“Form D1”), characterized by an X-ray powder diffraction (XRPD) pattern comprising peaks at 16.0, 16.5, 16.7, 20.0, and 20.4±0.2° 2θ using CuKα radiation.

48. The crystalline form of embodiment 47, further characterized by XRPD pattern peaks at 8.4, 12.1, 13.8, 14.2, 23.6, 24.6, and 25.2±0.2° 2θ using CuKα radiation.

49. A crystalline form of Compound A malonate salt (“Form E1”), characterized by an X-ray powder diffraction (XRPD) pattern comprising peaks at 6.8, 12.6, 16.6, 20.4, and 22.0±0.2° 2θ using CuKα radiation.

50. The crystalline form of embodiment 49, further characterized by XRPD pattern peaks at 5.4, 8.3, 17.2, 19.4, 21.0, 22.9, and 26.2±0.2° 2θ using CuKα radiation.

51. A crystalline form of Compound A naphthalene-2-sulfonate salt (“Form F1”), characterized by an X-ray powder diffraction (XRPD) pattern comprising peaks at 4.7, 14.7, 14.9, 17.0, 19.6, and 22.1±0.2° 2θ using CuKα radiation.

52. A crystalline form of Compound A naphthalene-2-sulfonate salt (“Form F2”), characterized by an X-ray powder diffraction (XRPD) pattern comprising peaks at 5.8, 11.6, 14.0, 17.5, and 19.7±0.2° 2θ using CuKα radiation.

53. A crystalline form of Compound A naphthalene-2-sulfonate salt (“Form F3”), characterized by an X-ray powder diffraction (XRPD) pattern comprising peaks at 3.8, 7.6, 9.7, 11.5, and 15.3±0.2° 2θ using CuKα radiation.

54. A crystalline form of Compound A methane sulfonate salt (“Form G1”), characterized by an X-ray powder diffraction (XRPD) pattern comprising peaks at 5.1, 6.5, 13.6, 13.8, and 19.5±0.2° 2θ using CuKα radiation.

55. The crystalline form of embodiment 54, further characterized by XRPD pattern peaks at 6.9, 9.3, 18.5, 20.8, and 21.5±0.2° 2θ using CuKα radiation.

56. A crystalline form of Compound A methane sulfonate salt (“Form G2”), characterized by an X-ray powder diffraction (XRPD) pattern comprising peaks at 5.6, 13.1, 13.3, 16.3, and 18.3±0.2° 2θ using CuKα radiation.

57. The crystalline form of embodiment 56, further characterized by XRPD pattern peaks at 12.5, 19.8, 21.5, and 9.4±0.2° 2θ using CuKα radiation.

58. A crystalline form of Compound A oxalate salt (“Form H2”), characterized by an X-ray powder diffraction (XRPD) pattern comprising peaks at 6.9, 8.7, 13.6, 17.4, and 24.6±0.2° 2θ using CuKα radiation.

59. The crystalline form of embodiment 58, further characterized by XRPD pattern peaks at 11.0, 19.6, 20.8, 21.0, 22.0, 25.3, and 27.3±0.2° 2θ using CuKα radiation.

60. A crystalline form of Compound A tartrate salt (“Form I1”), characterized by an X-ray powder diffraction (XRPD) pattern comprising peaks at 3.4, 14.7, 15.6, 18.0, and 24.3±0.2° 2θ using CuKα radiation.

61. The crystalline form of embodiment 60, further characterized by XRPD pattern peaks at 14.3, 15.1, 18.7, 19.4, and 19.7±0.2° 2θ using CuKα radiation.

62. The crystalline form of embodiment 60 or 61, further characterized by XRPD pattern peaks at 13.3, 13.5, 20.6, 21.1, 23.1, 23.5, 24.9, 26.7, and 27.2±0.2° 2θ using CuKα radiation.

63. A crystalline form of Compound A ethanesulfonate salt (“Form J1”), characterized by an X-ray powder diffraction (XRPD) pattern comprising peaks at 5.6, 7.9, 13.6, 15.7, and 17.9±0.2° 2θ using CuKα radiation.

64. The crystalline form of embodiment 63, further characterized by XRPD pattern peaks at 6.6, 11.1, 13.1, 16.8, 18.7, and 20.4±0.2° 2θ using CuKα radiation.

65. A crystalline form of Compound A ethanesulfonate salt (“Form J2”), characterized by an X-ray powder diffraction (XRPD) pattern comprising peaks at 4.0, 6.3, 7.7, 15.8, and 20.9±0.2° 2θ using CuKα radiation.

66. The crystalline form of embodiment 65, further characterized by XRPD pattern peaks at 7.9, 16.8, 18.4, 18.6, 19.5, and 20.0±0.2° 2θ using CuKα radiation.

67. The crystalline form of embodiment 65 or 66, further characterized by XRPD pattern peaks at 6.9, 17.6, 21.6, 23.3, 23.8, 24.1, 24.7, and 26.1±0.2° 2θ using CuKα radiation.

68. A crystalline form of Compound A cyclamate salt (“Form K1”), characterized by an X-ray powder diffraction (XRPD) pattern comprising peaks at 5.8, 14.0, 15.6, 16.7, and 28.1±0.2° 2θ using CuKα radiation.

69. A crystalline form of Compound A maleate salt (“Form L1”), characterized by an X-ray powder diffraction (XRPD) pattern comprising peaks at 5.7, 17.0, 17.5, 25.6, and 26.1±0.2° 2θ using CuKα radiation.

70. A crystalline form of Compound A phosphate salt (“Form M1”), characterized by an X-ray powder diffraction (XRPD) pattern comprising peaks at 5.4, 16.2, 20.3, and 22.5±0.2° 2θ using CuKα radiation.

71. The crystalline form of embodiment 70, further characterized by XRPD pattern peaks at 10.8, 12.3, 21.8, and 32.9±0.2° 2θ using CuKα radiation.

72. A crystalline form of Compound A phosphate salt (“Form M2”), characterized by an X-ray powder diffraction (XRPD) pattern comprising peaks at 7.1, 14.2, 14.9, 17.8, and 19.6±0.2° 2θ using CuKα radiation.

73. The crystalline form of embodiment 72, further characterized by XRPD pattern peaks at 8.9, 10.6, 10.9, 13.4, 16.4, 16.8, and 21.4±0.2° 2θ using CuKα radiation.

74. The crystalline form of embodiment 72 or 73, further characterized by XRPD pattern peaks at 3.6, 7.4, 21.0, 21.8, 23.0, 25.0, 25.4, 26.3, and 26.9±0.2° 2θ using CuKα radiation.

75. A crystalline form of Compound A phosphate salt (“Form M3”), characterized by an X-ray powder diffraction (XRPD) pattern comprising peaks at 7.5, 7.8, 14.8, 15.0, and 15.4±0.2° 2θ using CuKα radiation.

76. The crystalline form of embodiment 75, further characterized by XRPD pattern peaks at 9.0, 9.8, 11.6, 11.7, 18.3, 22.2, and 25.2±0.2° 2θ using CuKα radiation.

77. The crystalline form of embodiment 75 or 76, further characterized by XRPD pattern peaks at 3.9, 16.2, 27.2, and 27.2±0.2° 2θ using CuKα radiation.

78. A crystalline form of Compound A salicylic acid cocrystal (“Form CC-1A”), characterized by an X-ray powder diffraction (XRPD) pattern comprising peaks at 9.3, 5.9, 9.7. 6.0, and 13.9, 24.0±0.2° 2θ using CuKα radiation.

79. The crystalline form of embodiment 78, further characterized by XRPD pattern peaks at 13.1, 8.3, and 18.6±0.2° 2θ using CuKα radiation.

80. A crystalline form of Compound A salicylic acid cocrystal (“Form CC-2A”), characterized by an X-ray powder diffraction (XRPD) pattern comprising peaks at 8.2, 9.2, 16.5, 18.5, and 16.0±0.2° 2θ using CuKα radiation.

81. The crystalline form of embodiment 80, further characterized by XRPD pattern peaks at 17.2, 11.1, 3.2, 11.8, 24.6, and 25.9±0.2° 2θ using CuKα radiation.

82. A crystalline form of Compound A salicylic acid cocrystal (“Form CC-3A”), characterized by an X-ray powder diffraction (XRPD) pattern comprising peaks at 3.6, 12.6, 8.0, 14.4, and 7.2±0.2° 2θ using CuKα radiation.

83. The crystalline form of embodiment 82, further characterized by XRPD pattern peaks at 15.7, 11.2, 13.0, 21.5, 10.8, and 16.1±0.2° 2θ using CuKα radiation.

84. A crystalline form of Compound A salicylic acid cocrystal (“Form CC-4A”), characterized by an X-ray powder diffraction (XRPD) pattern comprising peaks at 10.3, 16.2, 9.9, 16.3, 19.9±0.2° 2θ using CuKα radiation.

85. The crystalline form of embodiment 84, further characterized by XRPD pattern peaks at 26.3, 27.0, 25.3, 18.0, 12.8, and 9.7±0.2° 2θ using CuKα radiation.

86. The crystalline form of embodiment 84 or 85, further characterized by XRPD pattern peaks at 28.5, 28.0, 24.8, 24.4, 23.4, and 22.3±0.2° 2θ using CuKα radiation.

87. The crystalline form of any one of embodiments 84-86, further characterized by 17.8, 16.2, 15.7, 15.6, 15.4, 13.6, and 13.4±0.2° 2θ using CuKα radiation.

88. A crystalline form of Compound A salicylic acid cocrystal (“Form CC-5A”), characterized by an X-ray powder diffraction (XRPD) pattern comprising peaks at 5.3, 17.8, 10.6, 18.3, and 15.9±0.2° 2θ using CuKα radiation.

89. The crystalline form of embodiment 88, further characterized by XRPD pattern peaks at 25.7, 15.5, 19.1, 28.7, 9.2, 12.2, 11.0, and 12.9±0.2° 2θ using CuKα radiation.

90. The crystalline form of embodiment 88 or 89, further characterized by XRPD pattern peaks at 21.3, 19.8, 20.7, 24.3, 13.2, 26.6, 27.2, and 11.3±0.2° 2θ using CuKα radiation.

91. A crystalline form of Compound A formic acid cocrystal (“Form CC-1B”), characterized by an X-ray powder diffraction (XRPD) pattern comprising peaks at 8.6, 4.6, 17.8, 17.4, and 23.0±0.2° 2θ using CuKα radiation.

92. The crystalline form of embodiment 91, further characterized by XRPD pattern peaks at 11.3, 14.8, 15.7, 16.5, 18.4, 19.3, 20.7, 24.9, and 26.8±0.2° 2θ using CuKα radiation.

93. A crystalline form of Compound A benzoic acid cocrystal (“Form CC-1C”), characterized by an X-ray powder diffraction (XRPD) pattern comprising peaks at 11.6, 16.1, 14.2, 3.9, and 19.8±0.2° 2θ using CuKα radiation.

94. The crystalline form of embodiment 93, further characterized by XRPD pattern peaks at 10.4, 10.6, 12.4, 14.5, 17.3, 18.3, and 19.4±0.2° 2θ using CuKα radiation.

95. The crystalline form of embodiment 93 or 94, further characterized by XRPD pattern peaks at 7.7, 8.8, 17.7, 21.7, 23.2, 26.3, and 26.7±0.2° 2θ using CuKα radiation.

96. A crystalline form of Compound A isobutyric acid cocrystal (“Form CC-1D”), characterized by an X-ray powder diffraction (XRPD) pattern comprising peaks at 5.6, 6.1, 13.1, 16.0, and 17.1±0.2° 2θ using CuKα radiation.

97. The crystalline form of embodiment 96, further characterized by XRPD pattern peaks at 6.5, 8.0, 8.6, 10.7, 11.1, 12.2, and 19.6±0.2° 2θ using CuKα radiation.

98. A crystalline form of Compound A isobutyric acid cocrystal (“Form CC-2D”), characterized by an X-ray powder diffraction (XRPD) pattern comprising peaks at 5.2, 5.5, 6.3, 10.3, and 12.6±0.2° 2θ using CuKα radiation.

99. The crystalline form of embodiment 98, further characterized by XRPD pattern peaks at 6.8, 12.1, 13.0, and 14.3±0.2° 2θ using CuKα radiation.

100. A crystalline form of Compound A caprylic acid cocrystal (“Form CC-1E”), characterized by an X-ray powder diffraction (XRPD) pattern comprising peaks at 4.8, 6.2, 6.5, 18.1, and 21.1=0.2° 2θ using CuKα radiation.

101. The crystalline form of embodiment 100, further characterized by XRPD pattern peaks at 15.6, 20.6, 21.4, 22.4, and 24.9±0.2° 2θ using CuKα radiation.

102. A crystalline form of Compound A sorbic acid cocrystal (“Form CC-1F”), characterized by an X-ray powder diffraction (XRPD) pattern comprising peaks at 8.2, 10.8, 11.4, 18.2, and 21.2±0.2° 2θ using CuKα radiation.

103. The crystalline form of embodiment 102, further characterized by XRPD pattern peaks at 5.4, 7.1, 8.9, 12.7, 13.4, 14.8, 17.7, 21.6, and 24.6±0.2° 2θ using CuKα radiation.

104. A crystalline form of Compound A saccharin cocrystal (“Form CC-1G”), characterized by an X-ray powder diffraction (XRPD) pattern comprising peaks at 5.1, 9.8, 10.1, 16.5, and 20.4±0.2° 2θ using CuKα radiation.

105. A crystalline form of Compound A succinic acid cocrystal (“Form CC-1H”), characterized by an X-ray powder diffraction (XRPD) pattern comprising peaks at 11.3, 11.5, 18.3, 19.0, and 20.6±0.2° 2θ using CuKα radiation.

106. The crystalline form of embodiment 105, further characterized by XRPD pattern peaks at 13.7, 24.2, 25.2, and 28.3±0.2° 2θ using CuKα radiation.

107. A crystalline form of Compound A succinic acid cocrystal (“Form CC-2H”), characterized by an X-ray powder diffraction (XRPD) pattern comprising peaks at 4.2, 5.3, 8.3, 9.0, and 9.2±0.2° 2θ using CuKα radiation.

108. A crystalline form of Compound A adipic acid cocrystal (“Form CC-1I”), characterized by an X-ray powder diffraction (XRPD) pattern comprising peaks at 7.7, 10.5, 18.5, 18.9, and 21.7±0.2° 2θ using CuKα radiation.

109. The crystalline form of embodiment 108, further characterized by XRPD pattern peaks at 5.3, 12.1, 20.7, 24.2, and 25.7±0.2° 2θ using CuKα radiation.

110. An amorphous form of Compound A free base

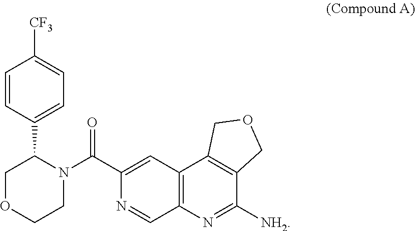 [SMILES: Nc1nc2cnc(C(=O)N3CCOC[C@@H]3c3ccc(C(F)(F)F)cc3)cc2c2c1COC2]

111. The amorphous form of embodiment 110, having a glass transition temperature (Tg) of 119° C.±3° C.

112. A pharmaceutical composition comprising the crystalline form or amorphous form of any one of embodiments 1 to 111 or a pharmaceutically acceptable salt thereof, and at least one pharmaceutically acceptable excipient.

113. The pharmaceutical composition of embodiment 112, wherein the composition is formulated for oral delivery.

114 The pharmaceutical composition of embodiment 112 or 113, wherein the composition is formulated for once-a-day administration.

115. The pharmaceutical composition of any one of embodiments 112-114, wherein the composition is an oral tablet.

116. The pharmaceutical composition of any one of embodiments 112-115, comprising 1-4000 mg of the crystalline form or amorphous form.

117. The pharmaceutical composition of embodiment 116 comprising 1-2000 mg of the compound or crystalline form.

118. A method of treating a cancer in a subject in need thereof comprising administering to the subject a therapeutically effective amount of the crystalline form or amorphous form of any one of embodiments 1 to 108 or a pharmaceutically acceptable salt thereof.

119. The method of embodiment 118, wherein the cancer is ovarian, lung, lymphoid, glioblastoma, colon, melanoma, gastric, pancreatic, or bladder cancer.

120. The method of embodiment 119, wherein the cancer is non-small cell lung cancer.

121 The method of embodiment 119, wherein the cancer is pancreatic cancer.

122. The method of any one of embodiments 118-121, wherein the cancer is a MTAP-null cancer.

123. The method of embodiment 122, wherein the cancer the MTAP-null cancer is selected from lung cancer, biliary tract cancer, head and neck squamous cell carcinoma, pancreatic adenocarcinoma, gallbladder cancer, and mesothelioma.

124. The method of embodiment 123, wherein the MTAP-null cancer is lung cancer.

125. The method of embodiment 124, wherein the lung cancer is non-squamous cell lung cancer (NSCLC).

126. The method of any one of embodiments 118-123, wherein the cancer is not a primary brain tumor or lymphoma.

127. The method of any one of embodiments 118-126, wherein the subject does not have, and has not had, interstitial lung disease or pneumonitis.

128. The method of any one of embodiments 118-127, wherein the crystalline form is crystalline Form 1.

### OTHER EMBODIMENTS

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the invention.

### Materials and Methods

Commercially available reagents are used as is without further purification unless specified.

The crystalline forms disclosed herein may be characterized using conventional means, including physical constants, diffraction data, and spectral data.

X-Ray Powder Diffraction—Method 1: X-ray powder diffraction (XRPD) data were obtained on a PANalytical X′Pert PRO X-ray diffraction system with RTMS detector. Samples were scanned at ambient temperature in a continuous mode from 5 to 45° or 3 to 40° (2θ) with step size of 0.0334° or 0.0167° respectively at a time per step of 50 s at 45 kV and 40 mA with CuKα radiation (1.541874 Å). Some X-ray powder diffraction (XRPD) data were also obtained on a Burker D8 Advance with Twin twin optic and Eiger detector. Samples were scanned at ambient temperature in a continuous mode from 3 to 40° (2θ) with step size of 0.02° at a 0.2 s time/step (7 min scan time) at 40 kV and 40 mA with Cu radiation (K1=1.5406 Å) line focus.

X-Ray Powder Diffraction—Method 2: In some instances, XRPD was performed with a Panalytical Aeris Powder XRPD on a Si zero-background holder. The 2θ position was calibrated against a Panalytical Si reference standard disc. The parameters used are listed in the table below.

### XRPD Parameters

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed 1/8° |
| Scan mode | Continuous |
| Scan range | 3-40 |
| (° 2θ) | |
| Scan step time [s] | 17.595 |
| Step size (° 2θ) | 0.0109 |
| Test Time | 4 min 30 s or 20 min |

X-ray Powder Diffraction—Method 3: Powder X-ray diffractograms were collected using a Bruker D8 Advance diffractometer equipped with a twin-twin optic and Eiger X-ray detector in reflection mode. The samples were scanned at ambient temperature in continuous mode from 3-45° 2θ with step size of 0.02° 2θ at 40 kV and 40 mA with CuKα radiation (1.54 Å). The incident beam path was equipped with a Nickel filter 0.2 mm and an anti-air scatter slit. Samples were prepared on a low background sample holder and placed on a spinning stage with a rotation time of 10 rev/min. Data were collected using DIFFRAC.MEASUREMENT CENTER (v. 7.5) and processed with DIFFRAC.EVA (v. 5.2).

Single Crystal Structure: A single crystal of high quality was mounted on a MiteGEN loop using mineral oil. Data were collected at 100 K on a Bruker-AXS X8 Kappa diffractometer coupled to a Bruker Photon 2 CPAD detector using Cu Kα radiation (λ=1.54178 Å) from an 1 μS microsource. All non-hydrogen atoms were refined anisotropically. All carbon-bound hydrogen atoms were placed in geometrically calculated positions and refined using a riding model while constraining their Uiso to 1.2 times the Ueq of the atoms to which they bind. Data reduction was carried out with the program SAINT and semi-empirical absorption correction based on equivalents was performed with the program SADABS.

Differential scanning calorimetry (DSC)—Method 1: DSC analysis was conducted on a TA Instruments Discovery Series calorimeter at 10° C./min from 30 to 300° C. in a crimped, aluminum T_{zero} pan under dry nitrogen at 50 mL/min using a nickel reference standard. Sample size was approximately 2-4 mg.

Thermal gravimetric analysis (TGA)—Method 1: TGA was performed on a TA Instruments Discovery Series analyzer at 10° C./min from ambient temperature to 300° C. in a platinum pan under dry nitrogen at 25 mL/min using an Indium reference standard. Sample size was approximately 5 mg. Detailed parameters used are listed in the table below.

### Parameters for Thermal Analysis by TGA and DSC

| Parameters | TGA | DSC |
|---|---|---|
| Method | Ramp | Ramp |
| Sample pan | Platinum, open | Aluminum |
| Temperature | RT - desired temperature | 25° C. - desired temperature |
| Heating rate | 10° C./min | 10° C./min |
| Purge gas | N₂ | N₂ |

Differential Scanning calorimetry—Method 2: Differential scanning calorimetery (DSC) analysis was conducted on a TA Instruments Q2000, Q1000, and/or Discovery Series calorimeter using a sample size of approximately 1-5 mg at 10° C./min from −5-30° C. to 250-350° C. in a crimped T_{zero} aluminum pan under dry nitrogen flow at 50 mL/min.

Alternatively, DSC was performed using a TA Instruments Q2000 differential scanning calorimeter. Temperature calibration was performed using NIST-traceable indium metal. The sample was placed into an aluminum Tzero pan, covered with a lid, crimped, and the weight was accurately recorded. A weighed aluminum pan configured as the sample pan was placed on the reference side of the cell. The data acquisition parameters and pan configuration for each thermogram are displayed in the image in the Data section of this report.

An illustrative procedure for amorphous form of Compound A fee base Form 4 mDSC was performed using the following procedure/settings: 1) equilibrate at 0.00° C.; 2) data storage is on; 3) modulate+/−0.50° C. every 60 s; 4) ramp 2.00° C./min to 26.00° C.; 5) mark end of cycle 1; and 6) end of method.

Thermal Gravimetric Analysis—Method 2: Thermal gravimetric analysis (TGA) was performed on a TA instruments Q5000, Q500, and/or Discovery Series analyzer using a sample size of approximately 1-5 mg at 10° C./min from ambient to 250-400° C. in a platinum pan under dry nitrogen at 25 mL/min.

DVS—Method 1: DVS was measured via a SMS (Surface Measurement Systems) DVS Resolution. The relative humidity at 25° C. was calibrated against the deliquescence point of LiCl, Mg(NO₃)₂ and KCl. Actual parameters for DVS are listed in the table below.

### Parameters for DVS

| Item | Value | |
|---|---|---|
| Temperature | 25° | C. |
| Sample size | 10-20 | mg |
| Gas and flow rate | N₂, 200 mL/min | |
| dm/dt | 0.002%/min | |
| Min. dm/dt stability duration | 10 | min |
| Max. equilibrium time | 180 | min |

| | |
|---|---|
| RH range | 30% RH-95% RH-0% RH-95% RH |
| RH step size | 10% |

DVS—Method 2: Moisture sorption data was collected using a DVS vapor sorption analyzer. A sample size of approximately 10-20 mg was used in a glass or metal pan. Hygroscopicity was evaluated from 40 to 90 to 0 to 90 to 40% RH in increments of 5% RH or from 40 to 0 to 95 to 0 to 95% RH in increments of 10% RH. Data for adsorption and desorption cycles were collected. Equilibrium criteria were set at 0.002% weight change in 1 minute with a maximum equilibration time of 180 minutes.

NMR: ¹³C solid-state NMR spectra were acquired at 11.7 T on a widebore Bruker Avance III spectrometer equipped with a 4 mm H/F/X magic angle spinning probe at 298 K. Chemical shifts were referenced externally using Teflon (PTFE) with the ¹⁹F resonance set to −123.2 ppm and with adamantane with the ¹³C CH₂ resonance set to 38.48 ppm. Approximately 100 mg of sample was packed into a 4 mm zirconia rotor and a magic angle spinning frequency of 14 kHz was used. The NMR data was processed using Topspin 3.6.4 or 3.5 software.

By way of example, the following parameters were used for Form 1: for ¹H-¹³C cross polarization (CP) a 64 KHz ¹H excitation pulse followed by a 70-100% amplitude ramped ¹H pulse and a constant amplitude 13C pulse were used with a contact time of 3 ms; during acquisition ¹H heteronuclear decoupling was achieved using SPINAL-64. 2048 transients were acquired for the spectrum using a recycle delay of 28 s; and for ¹⁹F experiments, a 45 kHz ¹⁹F excitation pulse was used, during acquisition ¹H heteronuclear decoupling was achieved with SPINAL-64, 256 transients were acquired for the spectrum using a recycle delay of 4.03 s.

### Example 1: Crystalline Compound A Form 1

Crystalline form of Compound A free base (Form 1) was first prepared by slurring Compound A in water at 37° C. By way of example, Form 1 can be prepared by slurry conversion of Form 8. Alternatively, Form 1 can be prepared by slurry conversion of free base amorphous form.

The following procedures illustrate various processes for preparing Form 1. Form 1 can be formed by a) slurry conversion at 55° C. for 8 hours in heptane, cyclohexane, 2-methyltetrahydrofuran, 1,2-dichloroethane/heptane (1:1), acetonitrile/heptane (1:1), water, isopropyl alcohol/water (1:4), ethanol/water (1:1), ethanol/water (1:4), acetonitrile/water (1:4), dimethylformamide/water (1:1), dimethylformamide/water (1:4); b) slow evaporation at room temperature from toluene, acetone, dioxane, dimethoxyethane, isopropyl acetate, or ethyl acetate; c) slurry conversion at room temperature for 5 days in acetone/water 40/60, acetone/water 20/80, acetonitrile/water 20/80, acetonitrile/water 10/90, ethanol/water 40/60, ethanol/water 50/50, ethanol/water 70/30, ethanol/water 80/20, ethanol/water 90/10, isopropyl alcohol/water 10/90, isopropyl alcohol/water 30/70, isopropyl alcohol/water 40/60, isopropyl alcohol/water 60/40, isopropyl alcohol/water 80/10, isopropyl alcohol/water 90/10; and/or. d) adding antisolvent to acetone, or DMSO.

The resulting solids were identified as crystalline Form 1 by XRPD as shown in Table 1 and FIG. 1. In addition, the DSC and DVS are shown in FIG. 2 and 3, respectively. As shown in FIG. 2, the DSC indicates a melt extrapolated onset at 225° C. and a weight loss of 0.03% from 35-150° C. and thermal decomposition above 250° C. As shown in FIG. 3, the DVS indicates Form 1 is non-hygroscopic between 0-95% relative humidity at 25° C. with no observable change in form.

TABLE 1
Form 1 XRPD Data

| Pos. [°2θ] | FWHM Left [°2θ] | d-spacing [Å] | Rel. Int. [%] |
|---|---|---|---|
| 4.5 | 0.12 | 19.61 | 63.52 |
| 6.7 | 0.09 | 13.29 | 0.57 |
| 9.0 | 0.15 | 9.84 | 100.00 |
| 12.2 | 0.09 | 7.24 | 2.47 |
| 12.5 | 0.09 | 7.10 | 2.56 |
| 12.8 | 0.09 | 6.94 | 0.71 |
| 13.3 | 0.12 | 6.65 | 18.69 |
| 13.7 | 0.09 | 6.44 | 0.89 |
| 14.3 | 0.09 | 6.18 | 1.57 |
| 14.7 | 0.12 | 6.02 | 11.06 |
| 15.7 | 0.12 | 5.66 | 7.25 |
| 16.2 | 0.12 | 5.46 | 17.15 |
| 16.7 | 0.12 | 5.31 | 11.28 |
| 17.4 | 0.06 | 5.10 | 3.20 |
| 17.6 | 0.12 | 5.03 | 7.26 |
| 18.0 | 0.09 | 4.92 | 0.93 |
| 18.4 | 0.12 | 4.83 | 1.74 |
| 18.8 | 0.12 | 4.72 | 18.86 |
| 19.6 | 0.12 | 4.54 | 2.07 |
| 20.0 | 0.12 | 4.44 | 2.29 |
| 20.3 | 0.09 | 4.38 | 4.87 |
| 20.8 | 0.09 | 4.27 | 0.94 |
| 21.2 | 0.12 | 4.20 | 4.36 |
| 21.6 | 0.15 | 4.12 | 1.91 |
| 22.2 | 0.09 | 4.01 | 1.60 |
| 22.5 | 0.21 | 3.96 | 12.85 |
| 23.0 | 0.15 | 3.86 | 4.14 |
| 23.8 | 0.12 | 3.74 | 3.32 |
| 24.1 | 0.12 | 3.70 | 1.04 |
| 24.6 | 0.18 | 3.61 | 0.90 |
| 25.1 | 0.18 | 3.55 | 3.85 |
| 26.2 | 0.12 | 3.41 | 7.28 |
| 26.4 | 0.12 | 3.37 | 15.66 |
| 26.8 | 0.12 | 3.32 | 5.86 |
| 27.8 | 0.18 | 3.21 | 3.74 |
| 28.4 | 0.18 | 3.15 | 0.80 |
| 28.8 | 0.18 | 3.10 | 0.42 |
| 29.7 | 0.12 | 3.01 | 0.83 |
| 30.5 | 0.12 | 2.93 | 0.92 |
| 30.9 | 0.09 | 2.89 | 0.73 |
| 32.7 | 0.18 | 2.74 | 0.25 |
| 34.4 | 0.18 | 2.61 | 0.42 |
| 35.1 | 0.15 | 2.55 | 1.48 |
| 35.6 | 0.18 | 2.52 | 0.61 |
| 36.4 | 0.15 | 2.45 | 1.32 |
| 37.8 | 0.09 | 2.38 | 0.82 |
| 39.4 | 0.12 | 2.29 | 2.17 |

In addition, single crystals of Free Base Form 1 were grown in ethyl acetate by slow evaporation at room temperature and used for single crystal X-ray structure determination.

TABLE 2
X-ray Single Structure Data

| Crystal System | Monoclinic | |
|---|---|---|
| Space Group | C2 | |
| Unit Cell | a = 40.410(2) Å | |
| | b = 7.0626(3) Å | |
| | c = 14.5109(6) Å | |
| | α = 90° | |
| | β = 106.722(3)° | |
| | γ = 90° | |
| Volume | 3966.2 | Å³ |
| Z(Z′) | 8 (2) | |
| Density (Mg/m3) | 1.488 | |
| Temperature | 100(2) | K |

¹³C ssNMR Data. ¹³C NMR isotropic peaks at: 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm. ¹⁹F ssNMR Data. −62.0 and −63.9 ppm at 25° C.

Crystalline Compound A Form 3 was prepared using any one of the following conditions: a) adding a solution of Compound A in 2-methyl tetrahydrofuran to heptane so that the volume ratio of 2-methyl tetrahydrofuran to heptane was 1:2 at room temperature; b) adding a solution of Compound A in tetrahydrofuran to methyl t-butyl ether so that the volume ratio of tetrahydrofuran to methyl t-butlyl ether was 1:2 at 5° C.; c) slow evaporation from methanol/methyl t-butyl ether (1:2); d) slow evaporation from dichloromethane at room temperature e) adding a solution of Compound A in dichloromethane to pentane so that the volume ratio of dichloromethane to pentrate was 1:2 at room temperature; f) formed by slurry conversion in dichloromethane at 5° C.; and g) formed by liquid vapor diffusion from a dioxane solution and antisolvent of methyl t- butyl ether at 5° C.; h) slow evaporation from methyl ethyl ketone/toluene (1:2); i) formed by liquid vapor diffusion from a 2-methyl tetrahydrofuran solution and antisolvent of pentane at room temperature j) slow evaporation from methyl t-butyl ether at room temperature. Form 3 was also formed by slow evaporation from dichloromethane at room temperature.

### Example 2: Crystalline Compound A Free Base Form 3

Crystalline Compound A Free Base Form 3 was prepared using any one of the following conditions: a) adding heptane as an antisolvent to a solution of Compound A in 2-methyl tetrahydrofuran/heptane (1:2) at room temperature; b) adding methyl t-butyl ether as an antisolvent to a solution of Compound A in tetrahydrofuran/methyl t-butyl ether (1:2) at 5° C.; c) evaporation from methanol/methyl t-butyl ether (1:2); d) evaporation from dichloromethane at room temperature e) adding pentane as an antisolvent to a solution of Compound A in dichloromethane/pentane (1:2) at room temperature; f) formed by slurry conversion in dichloromethane at 5° C.; and g) formed by liquid vapor diffusion from a dioxane solution and antisolvent of methyl t-butyl ether at 5° C. Form 3 was also formed by slow evaporation from dichloromethane at room temperature.

The resulting solids were identified as crystalline Form 3 by XRPD as shown in Table 3 and FIG. 4.

TABLE 3
Form 3 XRPD Data

| Pos. [°2θ] | FWHM [°2θ] | d-spacing [Å] | Rel. Int. [%] |
|---|---|---|---|
| 4.3 | 0.16 | 20.54 | 100 |
| 8.6 | 0.18 | 10.30 | 39.17 |
| 12.2 | 0.12 | 7.25 | 6.96 |
| 12.6 | 0.18 | 7.00 | 59.26 |
| 13.3 | 0.12 | 6.64 | 8.59 |
| 13.9 | 0.14 | 6.35 | 30.2 |
| 14.4 | 0.12 | 6.17 | 67.31 |
| 15.6 | 0.10 | 5.69 | 39.92 |
| 16.2 | 0.18 | 5.47 | 42.96 |
| 16.7 | 0.16 | 5.30 | 35.06 |
| 18.4 | 0.18 | 4.83 | 19.88 |
| 19.7 | 0.24 | 4.51 | 10.9 |
| 20.3 | 0.20 | 4.37 | 19.67 |
| 23.1 | 0.24 | 3.85 | 7.1 |
| 23.9 | 0.24 | 3.72 | 6.38 |
| 24.5 | 0.16 | 3.63 | 7.61 |
| 25.3 | 0.12 | 3.53 | 30.55 |
| 25.6 | 0.14 | 3.48 | 40.21 |
| 26.7 | 0.16 | 3.34 | 28.31 |
| 27.9 | 0.20 | 3.20 | 16.69 |
| 28.4 | 0.16 | 3.14 | 16.56 |

### Example 3: Crystalline Compound A Free Base Form 6

Crystalline Compound A Free Base Form 6 was prepared by slurrying Compound A in ethanol at room temperature. The resulting solids were identified as crystalline Form 6 by XRPD as shown in Table 4 and FIG. 7.

TABLE 4
Form 6 XRPD Data

| Pos. [°2θ] | FWHM [°2θ] | d-spacing [Å] | Rel. Int. [%] |
|---|---|---|---|
| 4.3 | 0.06 | 20.43 | 45.00 |
| 4.5 | 0.06 | 19.67 | 51.74 |
| 7.4 | 0.09 | 11.91 | 14.95 |
| 8.6 | 0.06 | 10.24 | 47.37 |
| 9.0 | 0.08 | 9.84 | 100.00 |
| 10.5 | 0.11 | 8.40 | 3.82 |
| 12.3 | 0.06 | 7.21 | 32.54 |
| 12.9 | 0.09 | 6.89 | 39.68 |
| 13.3 | 0.10 | 6.65 | 13.22 |
| 13.7 | 0.13 | 6.46 | 2.83 |
| 14.9 | 0.06 | 5.96 | 68.89 |
| 15.6 | 0.09 | 5.67 | 4.71 |
| 16.2 | 0.05 | 5.47 | 18.58 |
| 16.6 | 0.09 | 5.33 | 7.27 |
| 17.3 | 0.13 | 5.11 | 5.78 |
| 17.8 | 0.06 | 5.00 | 10.45 |
| 18.8 | 0.08 | 4.72 | 13.29 |
| 19.4 | 0.26 | 4.57 | 2.84 |
| 20.3 | 0.09 | 4.38 | 4.32 |
| 21.2 | 0.08 | 4.20 | 7.71 |
| 21.7 | 0.11 | 4.10 | 5.33 |
| 22.5 | 0.17 | 3.96 | 9.18 |
| 23.0 | 0.11 | 3.87 | 3.50 |
| 23.8 | 0.13 | 3.74 | 1.70 |
| 24.7 | 0.09 | 3.61 | 5.15 |
| 25.1 | 0.13 | 3.55 | 6.17 |
| 26.4 | 0.08 | 3.37 | 9.94 |
| 26.8 | 0.09 | 3.32 | 6.59 |
| 27.7 | 0.13 | 3.22 | 3.88 |
| 28.8 | 0.17 | 3.10 | 1.29 |
| 29.7 | 0.26 | 3.01 | 0.79 |
| 30.5 | 0.26 | 2.93 | 0.66 |
| 35.1 | 0.26 | 2.56 | 1.34 |
| 36.4 | 0.17 | 2.47 | 1.25 |
| 39.4 | 0.26 | 2.29 | 1.66 |

### Example 4: Crystalline Compound A Free Base Form 7

Crystalline Compound A Free Base Form 7 was prepared using the following conditions: a) slow evaporation from acetonitrile and pentane (1:2); b) slow evaporation from ethyl acetate and toluene (1:2); and c) slow evaporation from acetonitrile. The resulting solids were identified as crystalline Form 7 by XRPD as shown in Table 5 and FIG. 9.

TABLE 5
Form 7 XRPD Data

| Pos. [°2θ] | FWHM [°2θ] | d-spacing [Å] | Rel. Int. [%] |
|---|---|---|---|
| 4.3 | 0.05 | 20.69 | 100.00 |
| 8.5 | 0.08 | 10.36 | 43.55 |
| 9.6 | 0.13 | 9.23 | 5.47 |
| 11.2 | 0.30 | 7.90 | 2.91 |
| 12.3 | 0.15 | 7.22 | 33.26 |
| 13.1 | 0.08 | 6.78 | 26.89 |
| 13.7 | 0.13 | 6.45 | 6.43 |
| 14.8 | 0.17 | 6.00 | 43.25 |
| 15.5 | 0.17 | 5.73 | 16.92 |
| 17.2 | 0.13 | 5.16 | 8.57 |
| 19.2 | 0.17 | 4.64 | 12.16 |
| 20.9 | 0.30 | 4.26 | 11.74 |
| 22.2 | 0.35 | 4.00 | 4.14 |

### Example 5 Crystalline Compound A Free Base Form 8 Hydrate

Crystalline Compound A Free Base Form 8 hydrate was prepared by slow evaporation from acetonitrile and water. The resulting solids were identified as crystalline Form 8 hydrate by XRPD as shown in Table 6 and FIG. 11.

TABLE 6
Form 8 Hydrate XRPD Data

| Pos. [°2θ] | FWHM [°2θ] | d-spacing [Å] | Rel. Int. [%] |
|---|---|---|---|
| 4.0 | 0.26 | 22.38 | 100.00 |
| 6.1 | 0.35 | 14.47 | 14.24 |
| 7.7 | 0.35 | 11.42 | 29.19 |
| 8.0 | 0.13 | 11.11 | 30.08 |
| 10.0 | 0.52 | 8.88 | 11.32 |
| 12.3 | 0.17 | 7.21 | 28.97 |
| 13.7 | 0.17 | 6.47 | 13.94 |
| 15.1 | 0.22 | 5.89 | 21.38 |
| 17.0 | 0.43 | 5.21 | 18.76 |
| 18.4 | 0.26 | 4.83 | 15.51 |
| 19.9 | 0.43 | 4.47 | 15.24 |
| 22.2 | 0.35 | 4.01 | 7.51 |
| 23.7 | 0.52 | 3.75 | 6.75 |
| 24.8 | 0.22 | 3.59 | 8.84 |
| 27.6 | 0.43 | 3.23 | 3.90 |

### Example 6: Crystalline Compound a Ethanol Solvate (Form 2A)

Crystalline Compound A mono-ethanol solvate (Form 2A) was first prepared by slurring Compound A in ethanol at room temperature. The resulting solids were identified as crystalline by XRPD as shown in Table 7 and FIG. 13.

TABLE 7
Form 2A XRPD Data

| Pos. [°2θ] | FWHM [°2θ] | d-spacing [Å] | Rel. Int. [%] |
|---|---|---|---|
| 4.6 | 0.20 | 19.23 | 1.19 |
| 5.8 | 0.07 | 15.15 | 100.00 |
| 6.1 | 0.05 | 14.57 | 8.70 |
| 6.4 | 0.07 | 13.87 | 8.19 |
| 8.3 | 0.12 | 10.71 | 7.70 |
| 8.5 | 0.07 | 10.47 | 2.94 |
| 9.0 | 0.07 | 9.81 | 7.88 |
| 11.6 | 0.07 | 7.60 | 32.55 |
| 12.7 | 0.08 | 6.95 | 35.55 |
| 12.8 | 0.05 | 6.90 | 25.93 |
| 13.0 | 0.05 | 6.82 | 7.64 |
| 13.5 | 0.10 | 6.54 | 6.44 |
| 13.8 | 0.08 | 6.43 | 4.52 |
| 14.5 | 0.08 | 6.12 | 8.31 |
| 15.7 | 0.13 | 5.64 | 0.52 |
| 16.5 | 0.10 | 5.37 | 13.76 |
| 16.7 | 0.08 | 5.31 | 8.32 |
| 17.5 | 0.10 | 5.08 | 1.10 |
| 18.0 | 0.12 | 4.92 | 39.06 |
| 18.3 | 0.05 | 4.86 | 8.83 |
| 19.1 | 0.10 | 4.64 | 2.21 |
| 19.6 | 0.13 | 4.54 | 0.84 |
| 20.5 | 0.08 | 4.33 | 0.96 |
| 20.8 | 0.23 | 4.28 | 1.01 |
| 21.3 | 0.10 | 4.17 | 1.07 |
| 21.4 | 0.07 | 4.14 | 1.28 |
| 21.8 | 0.13 | 4.07 | 0.54 |
| 22.6 | 0.12 | 3.94 | 14.44 |
| 23.3 | 0.13 | 3.81 | 11.93 |
| 24.4 | 0.13 | 3.66 | 0.53 |
| 24.8 | 0.12 | 3.59 | 0.85 |
| 25.7 | 0.12 | 3.47 | 19.76 |
| 25.8 | 0.10 | 3.45 | 15.86 |
| 26.3 | 0.17 | 3.39 | 0.82 |
| 26.8 | 0.12 | 3.33 | 0.84 |
| 27.2 | 0.05 | 3.27 | 0.65 |
| 27.7 | 0.08 | 3.22 | 1.31 |
| 28.6 | 0.08 | 3.12 | 0.69 |
| 29.1 | 0.05 | 3.07 | 0.56 |
| 29.5 | 0.10 | 3.03 | 0.36 |
| 29.8 | 0.10 | 2.99 | 0.51 |
| 30.7 | 0.20 | 2.91 | 0.07 |
| 31.0 | 0.08 | 2.89 | 0.33 |
| 31.7 | 0.08 | 2.83 | 0.68 |
| 32.5 | 0.13 | 2.76 | 0.18 |
| 33.1 | 0.06 | 2.70 | 2.96 |
| 33.2 | 0.04 | 2.70 | 2.27 |
| 33.8 | 0.20 | 2.65 | 0.48 |
| 35.0 | 0.20 | 2.56 | 0.24 |
| 35.3 | 0.12 | 2.54 | 0.34 |
| 36.5 | 0.16 | 2.46 | 0.42 |
| 36.7 | 0.12 | 2.45 | 0.54 |
| 37.5 | 0.08 | 2.40 | 1.69 |
| 37.6 | 0.06 | 2.40 | 1.13 |
| 38.8 | 0.24 | 2.32 | 0.36 |
| 39.2 | 0.16 | 2.29 | 0.21 |
| 39.6 | 0.08 | 2.27 | 0.43 |

Single crystals of Form 2A were grown in ethanol at room temperature and used for single crystal X-ray structure determination.

TABLE 8
X-ray Single Structure Data

| Crystal System | Monoclinic |
|---|---|
| Space Group | P2₁ |
| Unit Cell | a = 14.0551(3) Å |
| | b = 5.4000(10) Å |
| | c = 14.9397(3) Å |
| | α = 90° |
| | β = 95.5093(8)° |
| | γ = 90° |
| Volume | 1128.65 Å³ |
| Z (Z′) | 2 (1) |
| Density | 1.443 |
| Temperature | 100(2) K |

### Example 3: Crystalline Compound A Isopropanol Solvate (Form 3A)

Crystalline Compound A isopropanol solvate (Form 3A) was first prepared by slurring Compound A in isopropanol at room temperature. The resulting solids were identified as crystalline Form 3A by XRPD as shown in Table 9 and FIG. 15.

TABLE 9
Form 3A XRPD Data

| Pos. [°2θ] | FWHM [°2θ] | d-spacing [Å] | Rel. Int. [%] |
|---|---|---|---|
| 4.2 | 0.10 | 20.92 | 1.20 |
| 5.6 | 0.17 | 15.66 | 87.40 |
| 6.4 | 0.13 | 13.76 | 15.06 |
| 8.2 | 0.17 | 10.75 | 21.78 |
| 8.8 | 0.13 | 10.08 | 13.72 |
| 9.2 | 0.15 | 9.65 | 10.53 |
| 10.2 | 0.13 | 8.69 | 9.49 |
| 11.2 | 0.15 | 7.92 | 22.13 |
| 12.5 | 0.10 | 7.08 | 20.92 |
| 12.8 | 0.13 | 6.92 | 56.46 |
| 13.2 | 0.13 | 6.74 | 9.83 |
| 13.6 | 0.13 | 6.51 | 18.34 |
| 14.1 | 0.08 | 6.28 | 2.95 |
| 14.3 | 0.10 | 6.20 | 5.05 |
| 16.4 | 0.15 | 5.40 | 57.47 |
| 17.5 | 0.17 | 5.06 | 100.00 |
| 18.6 | 0.15 | 4.78 | 22.42 |
| 19.2 | 0.12 | 4.62 | 1.65 |
| 19.7 | 0.20 | 4.52 | 3.39 |
| 20.0 | 0.12 | 4.43 | 6.12 |
| 20.4 | 0.10 | 4.35 | 3.21 |
| 20.7 | 0.12 | 4.29 | 6.72 |
| 21.0 | 0.15 | 4.23 | 10.41 |
| 21.8 | 0.20 | 4.08 | 19.84 |
| 22.4 | 0.15 | 3.98 | 14.91 |
| 22.9 | 0.17 | 3.89 | 16.12 |
| 23.4 | 0.10 | 3.80 | 3.83 |
| 24.0 | 0.13 | 3.71 | 5.04 |
| 24.5 | 0.18 | 3.64 | 12.99 |
| 25.1 | 0.17 | 3.55 | 25.91 |
| 25.6 | 0.07 | 3.47 | 7.61 |
| 25.9 | 0.13 | 3.44 | 11.32 |
| 26.2 | 0.07 | 3.40 | 6.74 |
| 26.5 | 0.08 | 3.36 | 4.43 |
| 27.3 | 0.12 | 3.27 | 5.07 |
| 27.8 | 0.13 | 3.21 | 2.11 |
| 28.2 | 0.15 | 3.16 | 6.63 |
| 28.9 | 0.13 | 3.09 | 1.30 |
| 29.7 | 0.08 | 3.01 | 2.74 |
| 30.9 | 0.18 | 2.90 | 5.30 |
| 31.4 | 0.13 | 2.85 | 1.21 |
| 31.7 | 0.07 | 2.82 | 2.22 |
| 32.2 | 0.17 | 2.78 | 0.94 |
| 33.1 | 0.17 | 2.71 | 2.25 |
| 33.6 | 0.20 | 2.67 | 0.39 |
| 34.9 | 0.17 | 2.57 | 0.35 |
| 35.9 | 0.13 | 2.50 | 0.92 |
| 37.0 | 0.18 | 2.43 | 2.49 |
| 39.2 | 0.20 | 2.30 | 0.12 |

### Example 4: Crystalline Compound A Acetone Solvate (Form 4A)

Crystalline compound A acetone solvate Form 4A was prepared by slurry conversion of Compound A in acetone at 5° C. and at room temperature. The resulting solids were identified as crystalline Form 4A by XRPD as shown in Table 10 and FIG. 17.

TABLE 10
Form 4A XRPD Data

| Pos. [°2θ] | FWHM [°2θ] | d-spacing [Å] | Rel. Int. [%] |
|---|---|---|---|
| 4.3 | 0.13 | 20.47 | 13.34 |
| 5.7 | 0.10 | 15.40 | 100.00 |
| 7.3 | 0.15 | 12.08 | 12.34 |
| 7.7 | 0.19 | 11.50 | 20.36 |
| 8.6 | 0.17 | 10.34 | 7.80 |
| 10.0 | 0.43 | 8.83 | 3.16 |
| 11.2 | 0.13 | 7.90 | 10.44 |
| 11.5 | 0.17 | 7.67 | 17.45 |
| 12.9 | 0.13 | 6.86 | 7.98 |
| 13.4 | 0.19 | 6.63 | 13.28 |
| 14.2 | 0.22 | 6.26 | 8.64 |
| 14.8 | 0.15 | 5.99 | 18.98 |
| 15.3 | 0.17 | 5.79 | 27.81 |
| 16.2 | 0.17 | 5.46 | 13.13 |
| 17.3 | 0.26 | 5.11 | 9.03 |
| 18.5 | 0.15 | 4.79 | 10.45 |
| 19.6 | 0.30 | 4.52 | 10.32 |
| 20.4 | 0.35 | 4.35 | 11.78 |
| 21.3 | 0.43 | 4.17 | 8.44 |
| 24.0 | 0.17 | 3.71 | 14.80 |
| 26.5 | 0.52 | 3.36 | 6.98 |
| 27.5 | 0.26 | 3.24 | 4.39 |
| 28.3 | 0.26 | 3.15 | 3.52 |
| 30.0 | 0.43 | 2.98 | 2.99 |
| 32.8 | 0.52 | 2.73 | 0.53 |
| 34.8 | 0.69 | 2.58 | 0.77 |

### Example 5: Crystalline Compound A Methanol Solvate (Form 5A)

Crystalline compound A methanol solvate Form 5A was prepared by slurry conversion of Compound A in methanol at 5° C. and at room temperature. Form 5A was also prepared by slowly evaporating Compound A from methanol at room temperature. The resulting solids were identified as crystalline Form 5A by XRPD as shown in Table 11 and FIG. 19.

TABLE 11
Form 5A XRPD Data

| Pos. [°2θ] | FWHM [°2θ] | d-spacing [Å] | Rel. Int. [%] |
|---|---|---|---|
| 3.9 | 0.15 | 22.87 | 22.61 |
| 4.8 | 0.12 | 18.25 | 100.00 |
| 7.7 | 0.12 | 11.56 | 54.23 |
| 8.9 | 0.12 | 9.97 | 41.65 |
| 9.7 | 0.10 | 9.16 | 17.14 |
| 10.5 | 0.06 | 8.40 | 37.81 |
| 11.7 | 0.13 | 7.55 | 11.95 |
| 12.3 | 0.08 | 7.20 | 77.28 |
| 13.1 | 0.10 | 6.78 | 40.25 |
| 13.4 | 0.13 | 6.60 | 44.57 |
| 14.1 | 0.13 | 6.27 | 15.85 |
| 14.5 | 0.13 | 6.11 | 43.77 |
| 15.0 | 0.13 | 5.90 | 28.49 |
| 15.3 | 0.13 | 5.78 | 52.25 |
| 16.1 | 0.13 | 5.52 | 55.75 |
| 16.9 | 0.13 | 5.25 | 9.80 |
| 17.3 | 0.08 | 5.13 | 9.55 |
| 17.6 | 0.13 | 5.04 | 29.91 |
| 18.7 | 0.10 | 4.76 | 19.74 |
| 19.1 | 0.09 | 4.64 | 10.76 |
| 19.6 | 0.13 | 4.54 | 14.02 |
| 20.1 | 0.15 | 4.43 | 18.90 |
| 20.4 | 0.18 | 4.35 | 11.98 |
| 21.2 | 0.13 | 4.20 | 10.43 |
| 21.5 | 0.18 | 4.13 | 21.13 |
| 21.8 | 0.13 | 4.07 | 10.28 |
| 22.3 | 0.10 | 3.98 | 10.73 |
| 22.7 | 0.10 | 3.92 | 17.53 |
| 23.5 | 0.15 | 3.79 | 8.32 |
| 24.3 | 0.15 | 3.66 | 5.94 |
| 24.7 | 0.15 | 3.60 | 18.36 |
| 25.0 | 0.06 | 3.56 | 38.14 |
| 26.0 | 0.20 | 3.43 | 22.55 |
| 26.6 | 0.26 | 3.35 | 20.21 |
| 27.9 | 0.20 | 3.20 | 6.35 |
| 29.2 | 0.15 | 3.05 | 3.72 |
| 30.3 | 0.15 | 2.95 | 3.26 |
| 30.9 | 0.26 | 2.89 | 1.60 |
| 31.9 | 0.31 | 2.81 | 1.15 |

### Example 6: Amorphous Compound A Free Base (Form 4AA)

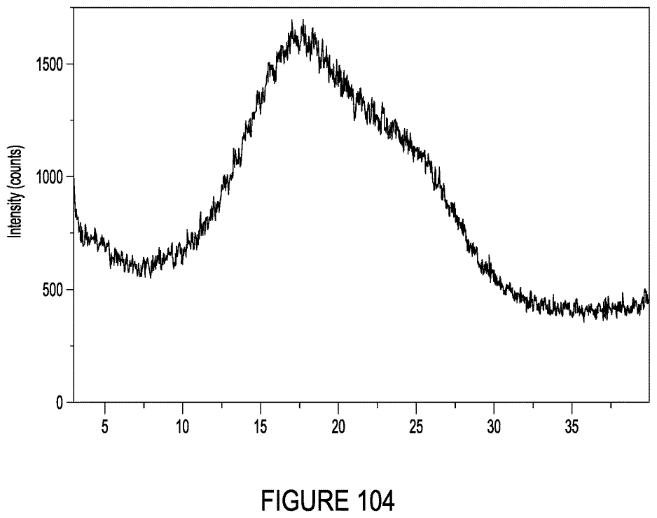

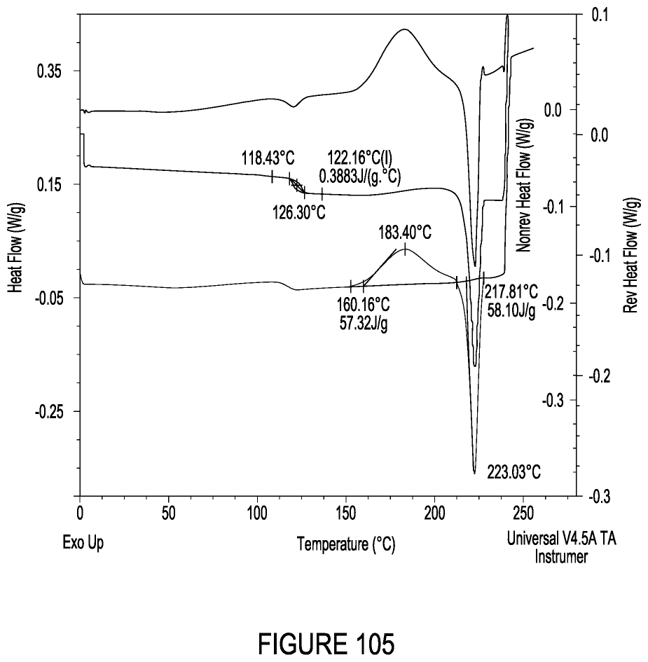

Amorphous Compound A free base (Form 4AA) was formed upon cooling of melts at 300° C. The resulting solids were evaluated using XRPD (FIG. 104) and DSC (FIG. 105).

Amorphous form Compound A free base can be formed upon cooling of a melt of Compound A free base under suitable conditions. For example, in some embodiments Compound A free base Form 1 can be heated at a rate of 20° C./min to a temperature of 300° C. and melted in a hot stage microscope, upon which a sample can be removed and rapidly cooled to room temperature.

The DSC was performed in a sealed crimped pan heated at 2° C./min with a modulation of +0.5° C. every 60 seconds, and showed a glass transition (Tg) at approximately 1189° C.

### Example 7: Crystalline Compound A Toluene Sulfonate (Form A1)

Crystalline Compound A toluene sulfonate (Form A1) was prepared by slurring one equivalent of toluene sulfonic acid and Compound A in a 50/50 toluene/methanol solvent mixture at ambient conditions. The resulting solids were identified as crystalline by XRPD as shown in Table 12 and FIG. 21.

TABLE 12
Form A1 XRPD Data

| Pos. [°2θ] | FWHM [°2θ] | d-spacing [Å] | Rel. Int. [%] |
|---|---|---|---|
| 6.0 | 0.19 | 14.78 | 100.00 |
| 9.5 | 0.19 | 9.28 | 32.53 |
| 10.2 | 0.19 | 8.68 | 2.91 |
| 11.9 | 0.19 | 7.43 | 21.40 |
| 14.3 | 0.19 | 6.19 | 16.91 |
| 14.8 | 0.16 | 6.01 | 4.55 |
| 15.1 | 0.13 | 5.87 | 1.56 |
| 15.5 | 0.16 | 5.72 | 1.65 |
| 16.0 | 0.19 | 5.55 | 7.42 |
| 17.1 | 0.19 | 5.17 | 7.21 |
| 17.9 | 0.19 | 4.97 | 32.44 |
| 19.1 | 0.19 | 4.66 | 71.67 |
| 20.3 | 0.19 | 4.37 | 46.13 |
| 21.4 | 0.19 | 4.168 | 3.29 |
| 21.9 | 0.16 | 4.06 | 9.77 |
| 22.5 | 0.16 | 3.96 | 0.73 |
| 22.9 | 0.19 | 3.88 | 0.79 |
| 23.4 | 0.19 | 3.80 | 3.43 |
| 24.1 | 0.19 | 3.69 | 46.27 |
| 24.9 | 0.19 | 3.57 | 59.92 |
| 25.8 | 0.16 | 3.45 | 2.97 |
| 26.2 | 0.19 | 3.40 | 18.50 |
| 27.9 | 0.19 | 3.20 | 10.78 |
| 28.9 | 0.23 | 3.09 | 87.89 |
| 29.6 | 0.16 | 3.02 | 6.27 |
| 30.0 | 0.16 | 2.98 | 2.62 |
| 31.4 | 0.13 | 2.85 | 1.70 |
| 32.2 | 0.16 | 2.78 | 6.36 |
| 32.5 | 0.16 | 2.76 | 5.26 |
| 33.8 | 0.19 | 2.65 | 18.84 |
| 34.4 | 0.19 | 2.61 | 2.41 |
| 35.4 | 0.19 | 2.54 | 20.82 |
| 36.9 | 0.16 | 2.44 | 6.18 |
| 37.4 | 0.10 | 2.40 | 1.09 |
| 38.3 | 0.13 | 2.35 | 3.31 |
| 38.6 | 0.16 | 2.33 | 3.51 |
| 39.3 | 0.16 | 2.29 | 1.33 |
| 40.2 | 0.23 | 2.24 | 7.99 |
| 41.3 | 0.13 | 2.19 | 9.21 |
| 41.5 | 0.19 | 2.18 | 12.94 |
| 42.0 | 0.23 | 2.15 | 3.65 |
| 42.8 | 0.19 | 2.11 | 6.60 |

### Example 8: Crystalline Compound A Toluene Sulfonate Salt (Form A2)

Crystalline Compound A toluene sulfonate (Form A2) was prepared by slurrying 1:1 molar ratio of Compound A and counterion toluene sulfonic in 2-methyltetrahydrofuran, acetone, or ethyl acetate at ambient conditions. The resulting solids were identified as crystalline by XRPD as shown in Table 13 and FIG. 24.

TABLE 13
Form A2 XRPD Data

| Pos. [°2θ] | FWHM [°2θ] | d-spacing [Å] | Rel. Int. [%] |
|---|---|---|---|
| 5.5 | 0.13 | 16.17 | 1.02 |
| 6.9 | 0.10 | 12.76 | 1.35 |
| 7.9 | 0.10 | 11.22 | 5.90 |
| 8.5 | 0.19 | 10.43 | 1.35 |
| 10.2 | 0.13 | 8.64 | 42.41 |
| 12.7 | 0.13 | 7.00 | 88.49 |
| 13.1 | 0.13 | 6.74 | 4.55 |
| 13.5 | 0.13 | 6.56 | 15.50 |
| 13.9 | 0.13 | 6.37 | 12.52 |
| 14.3 | 0.13 | 6.19 | 7.55 |
| 15.1 | 0.13 | 5.87 | 4.78 |
| 15.5 | 0.13 | 5.72 | 68.65 |
| 16.2 | 0.16 | 5.48 | 73.66 |
| 16.8 | 0.10 | 5.28 | 3.02 |
| 17.1 | 0.10 | 5.19 | 0.78 |
| 17.4 | 0.13 | 5.10 | 14.38 |
| 18.0 | 0.16 | 4.94 | 3.77 |
| 18.7 | 0.16 | 4.76 | 100.00 |
| 19.2 | 0.10 | 4.63 | 2.04 |
| 19.7 | 0.13 | 4.50 | 60.82 |
| 20.2 | 0.16 | 4.39 | 21.57 |
| 20.8 | 0.13 | 4.28 | 10.40 |
| 21.3 | 0.16 | 4.18 | 11.78 |
| 21.8 | 0.16 | 4.08 | 60.28 |
| 22.5 | 0.16 | 3.95 | 19.52 |
| 22.8 | 0.10 | 3.90 | 3.93 |
| 23.2 | 0.16 | 3.83 | 59.26 |
| 23.8 | 0.13 | 3.74 | 3.18 |
| 24.1 | 0.13 | 3.69 | 29.98 |
| 24.6 | 0.19 | 3.62 | 32.59 |
| 25.1 | 0.10 | 3.54 | 1.92 |
| 25.4 | 0.13 | 3.50 | 4.75 |
| 25.9 | 0.13 | 3.44 | 11.82 |
| 26.3 | 0.16 | 3.39 | 26.07 |
| 27.2 | 0.16 | 3.27 | 46.56 |
| 27.7 | 0.10 | 3.22 | 9.89 |
| 28.0 | 0.10 | 3.19 | 5.21 |
| 28.3 | 0.13 | 3.15 | 9.35 |
| 28.9 | 0.13 | 3.10 | 7.60 |
| 29.8 | 0.16 | 3.00 | 6.59 |
| 30.5 | 0.13 | 2.93 | 16.75 |
| 30.8 | 0.13 | 2.91 | 12.19 |
| 31.9 | 0.10 | 2.80 | 6.34 |
| 32.1 | 0.10 | 2.79 | 7.94 |
| 32.4 | 0.13 | 2.77 | 5.07 |
| 32.9 | 0.10 | 2.72 | 5.72 |
| 33.2 | 0.13 | 2.70 | 6.58 |
| 33.7 | 0.13 | 2.66 | 10.49 |
| 34.2 | 0.16 | 2.63 | 3.10 |
| 35.2 | 0.16 | 2.55 | 7.46 |
| 35.9 | 0.13 | 2.50 | 2.78 |
| 36.7 | 0.26 | 2.45 | 6.27 |
| 37.3 | 0.10 | 2.41 | 3.21 |
| 38.5 | 0.13 | 2.34 | 4.11 |
| 38.9 | 0.16 | 2.32 | 2.35 |
| 39.3 | 0.16 | 2.29 | 7.11 |
| 39.6 | 0.08 | 2.27 | 8.32 |
| 39.8 | 0.19 | 2.26 | 11.66 |
| 40.3 | 0.13 | 2.24 | 4.27 |
| 41.2 | 0.19 | 2.19 | 12.03 |
| 41.9 | 0.26 | 2.15 | 3.09 |
| 43.3 | 0.10 | 2.09 | 4.84 |
| 43.9 | 0.26 | 2.06 | 0.52 |
| 44.6 | 0.13 | 2.03 | 3.48 |

### Example 9: Crystalline Compound A Toluene Sulfonate Salt (Form A3)

Crystalline Compound A toluene sulfonate (Form A3) was prepared by reactive crystallization at room temperature with p-toluene sulfonic acid in acetone/heptane (1:1) with a 1:1 molar ratio of Compound A and counterion. It was also formed by reactive crystallization at room temperature with p-toluene sulfonic acid in methyl isobutyl ketone and dioxane with a 1:1 molar ratio of Compound A and counterion. The resulting solids were identified as crystalline Form A3 by XRPD as shown in Table 14 and FIG. 26.

TABLE 14
Form A3 XRPD Data

| Pos. [°2θ] | FWHM [°2θ] | d-spacing [Å] | Rel. Int. [%] |
|---|---|---|---|
| 4.9 | 0.18 | 18.07 | 100.00 |
| 10.0 | 0.17 | 8.89 | 0.53 |
| 14.7 | 0.17 | 6.05 | 1.62 |
| 15.2 | 0.09 | 5.83 | 1.99 |
| 15.7 | 0.17 | 5.64 | 0.51 |
| 16.7 | 0.43 | 5.31 | 0.47 |
| 17.5 | 0.22 | 5.06 | 0.59 |
| 18.2 | 0.17 | 4.86 | 1.45 |
| 18.8 | 0.19 | 4.73 | 2.53 |
| 19.5 | 0.11 | 4.54 | 2.71 |
| 20.0 | 0.15 | 4.45 | 1.17 |
| 22.2 | 0.17 | 4.00 | 0.52 |
| 23.4 | 0.15 | 3.80 | 1.01 |
| 24.5 | 0.24 | 3.63 | 3.20 |
| 27.2 | 0.26 | 3.27 | 0.27 |
| 28.2 | 0.35 | 3.16 | 0.58 |
| 33.1 | 0.35 | 2.71 | 0.29 |
| 34.5 | 0.26 | 2.60 | 0.39 |

### Example 10: Crystalline Compound A Benzene Sulfonate (Form B1)

Crystalline Compound A benzene sulfonate (Form B1) was prepared by cooling 1:1 molar ratio of Compound A and counterion benzene sulfonic acid in 50:50 toluene: methanol solvent mixture. It was also formed by reactive crystallization at room temperature in ethanol, methyl isobutyl ketone, dioxane, acetone/heptane (1:1) or dichloromethane with a 1:1 molar ratio of Compound A and counterion. It was also formed by slow evaporation at room temperature in acetonitrile/water (1:1). The resulting solids were identified as crystalline Form B1 by XRPD as shown in Table 15 and FIG. 29.

TABLE 15
Form B1 XRPD Data

| Pos. [°2θ] | FWHM [°2θ] | d-spacing [Å] | Rel. Int. [%] |
|---|---|---|---|
| 5.1 | 0.10 | 17.32 | 100.00 |
| 8.0 | 0.52 | 11.10 | 1.05 |
| 10.1 | 0.19 | 8.78 | 1.73 |
| 13.4 | 0.13 | 6.62 | 7.62 |
| 14.5 | 0.13 | 6.12 | 9.74 |
| 15.1 | 0.19 | 5.88 | 5.08 |
| 16.1 | 0.13 | 5.50 | 54.35 |
| 16.8 | 0.16 | 5.29 | 2.75 |
| 17.2 | 0.13 | 5.15 | 3.32 |
| 17.9 | 0.13 | 4.97 | 32.86 |
| 19.0 | 0.16 | 4.66 | 22.85 |
| 20.2 | 0.16 | 4.40 | 13.92 |
| 20.6 | 0.16 | 4.32 | 13.03 |
| 21.7 | 0.13 | 4.09 | 3.18 |
| 22.0 | 0.13 | 4.03 | 4.44 |
| 22.6 | 0.16 | 3.93 | 8.30 |
| 23.2 | 0.13 | 3.84 | 16.44 |
| 23.7 | 0.13 | 3.75 | 3.13 |
| 24.8 | 0.13 | 3.59 | 9.27 |
| 25.2 | 0.19 | 3.53 | 22.92 |
| 26.0 | 0.16 | 3.43 | 12.60 |
| 27.0 | 0.13 | 3.31 | 12.37 |
| 27.8 | 0.10 | 3.21 | 2.79 |
| 28.2 | 0.23 | 3.17 | 5.91 |
| 29.0 | 0.16 | 3.08 | 7.17 |
| 30.0 | 0.16 | 2.98 | 5.44 |
| 30.4 | 0.13 | 2.94 | 12.99 |
| 32.5 | 0.78 | 2.75 | 0.71 |
| 34.2 | 0.19 | 2.62 | 1.51 |
| 35.5 | 0.16 | 2.53 | 4.99 |
| 38.5 | 0.32 | 2.34 | 1.50 |
| 40.1 | 0.16 | 2.25 | 1.87 |
| 40.9 | 0.26 | 2.21 | 2.61 |
| 42.6 | 0.52 | 2.12 | 1.52 |

### Example 11: Crystalline Compound A Chloride Salt (Form C1)

Crystalline Compound A chloride salt (Form C1) was prepared by slurrying 1:1 molar ratio of Compound A and counterion HCl in isopropanol at ambient conditions. The resulting solids were identified as crystalline Form C1 by XRPD as shown in Table 16 and FIG. 31.

TABLE 16
chloride Form C1 XRPD Data

| Pos. [°2θ] | FWHM [°2θ] | d-spacing [Å] | Rel. Int. [%] |
|---|---|---|---|
| 6.8 | 0.19 | 12.98 | 18.29 |
| 8.5 | 0.23 | 10.42 | 3.60 |
| 9.9 | 0.26 | 8.93 | 0.73 |
| 11.9 | 0.19 | 7.45 | 18.42 |
| 13.5 | 0.19 | 6.57 | 7.46 |
| 16.2 | 0.13 | 5.48 | 9.20 |
| 16.6 | 0.19 | 5.36 | 100.00 |
| 17.3 | 0.16 | 5.13 | 3.30 |
| 19.2 | 0.23 | 4.61 | 1.29 |
| 19.7 | 0.26 | 4.50 | 8.75 |
| 20.7 | 0.29 | 4.29 | 30.06 |
| 21.4 | 0.26 | 4.15 | 5.72 |
| 22.2 | 0.23 | 4.01 | 3.23 |
| 22.6 | 0.19 | 3.93 | 2.06 |
| 23.3 | 0.23 | 3.82 | 2.02 |
| 23.8 | 0.26 | 3.74 | 11.63 |
| 24.8 | 0.26 | 3.59 | 7.02 |
| 25.3 | 0.23 | 3.53 | 14.96 |
| 27.6 | 0.26 | 3.24 | 17.75 |
| 29.0 | 0.19 | 3.08 | 2.54 |
| 29.8 | 0.16 | 3.00 | 1.48 |
| 30.2 | 0.16 | 2.96 | 3.76 |
| 30.6 | 0.19 | 2.93 | 3.75 |
| 31.8 | 0.10 | 2.82 | 0.69 |
| 32.2 | 0.16 | 2.78 | 2.31 |
| 32.5 | 0.16 | 2.75 | 1.90 |
| 33.3 | 0.19 | 2.69 | 0.31 |
| 34.1 | 0.23 | 2.63 | 2.04 |
| 34.8 | 0.26 | 2.58 | 3.25 |
| 35.3 | 0.19 | 2.54 | 2.23 |
| 35.9 | 0.19 | 2.50 | 0.63 |
| 36.5 | 0.19 | 2.46 | 0.93 |
| 37.8 | 0.26 | 2.38 | 2.58 |
| 39.9 | 0.26 | 2.26 | 0.47 |
| 41.9 | 0.16 | 2.16 | 3.57 |
| 42.6 | 0.19 | 2.12 | 0.59 |
| 43.4 | 0.16 | 2.09 | 1.19 |

### Example 12: Crystalline Compound A Chloride Salt (Form C2)

Crystalline Compound A chloride salt (Form C2) was prepared by reactive crystallization at room temperature with HCl in methyl isobutyl ketone with a 1:1 molar ratio of Compound A and counterion. The resulting solids were identified as crystalline Form C1 by XRPD as shown in Table 17 and FIG. 33.

TABLE 17
Form C2 XRPD Data

| Pos. [°2θ] | FWHM [°2θ] | d-spacing [Å] | Rel. Int. [%] |
|---|---|---|---|
| 4.2 | 0.13 | 21.20 | 100.00 |
| 5.6 | 0.13 | 15.70 | 70.35 |
| 6.8 | 0.13 | 12.99 | 32.82 |
| 7.1 | 0.17 | 12.41 | 31.31 |
| 7.8 | 0.22 | 11.29 | 17.56 |
| 8.7 | 0.17 | 10.18 | 16.36 |
| 9.4 | 0.13 | 9.38 | 34.05 |
| 10.9 | 0.17 | 8.09 | 21.03 |
| 11.8 | 0.13 | 7.50 | 24.02 |
| 12.2 | 0.35 | 7.24 | 58.31 |
| 12.9 | 0.17 | 6.89 | 50.05 |
| 13.4 | 0.13 | 6.63 | 48.57 |
| 14.0 | 0.26 | 6.32 | 37.19 |
| 14.9 | 0.22 | 5.94 | 19.18 |
| 15.4 | 0.17 | 5.74 | 27.20 |
| 16.5 | 0.35 | 5.36 | 32.82 |
| 17.7 | 0.17 | 5.01 | 37.84 |
| 18.1 | 0.22 | 4.90 | 64.28 |
| 19.1 | 0.15 | 4.65 | 21.67 |
| 20.5 | 0.26 | 4.33 | 4.41 |
| 24.8 | 0.39 | 3.59 | 15.81 |
| 27.4 | 0.17 | 3.26 | 12.16 |

### Example 13: Crystalline Compound A Sulfate Salt (Form D1)

Crystalline Compound A sulfate salt (Form D1) was prepared by slurrying 1:1 molar ratio of Compound A and counterion sulfuric acid in isopropanol at ambient conditions. The resulting solids were identified as crystalline Form D1 by XRPD as shown in Table 18 and FIG. 35.

TABLE 18
Form D1 XRPD Data

| Pos. [°2θ] | FWHM [°2θ] | d-spacing [Å] | Rel. Int. [%] |
|---|---|---|---|
| 6.6 | 0.23 | 13.45 | 15.05 |
| 8.4 | 0.13 | 10.50 | 31.82 |
| 12.1 | 0.10 | 7.30 | 27.72 |
| 13.0 | 0.16 | 6.79 | 6.94 |
| 13.8 | 0.23 | 6.43 | 23.15 |
| 14.2 | 0.10 | 6.24 | 20.12 |
| 14.8 | 0.32 | 5.97 | 3.95 |
| 16.0 | 0.16 | 5.54 | 100.00 |
| 16.5 | 0.13 | 5.37 | 41.34 |
| 16.7 | 0.10 | 5.30 | 57.07 |
| 20.0 | 0.16 | 4.45 | 85.42 |
| 20.4 | 0.23 | 4.35 | 53.91 |
| 21.5 | 0.13 | 4.14 | 13.13 |
| 22.2 | 0.32 | 4.00 | 17.20 |
| 23.6 | 0.23 | 3.77 | 22.56 |
| 24.2 | 0.26 | 3.68 | 8.03 |
| 24.6 | 0.16 | 3.61 | 20.81 |
| 25.2 | 0.26 | 3.54 | 22.17 |
| 26.4 | 0.16 | 3.38 | 6.62 |
| 26.9 | 0.19 | 3.31 | 5.98 |
| 29.7 | 0.26 | 3.01 | 7.85 |
| 32.2 | 0.19 | 2.78 | 8.35 |
| 38.4 | 0.19 | 2.34 | 5.52 |

### Example 14: Crystalline Compound A Malonate Salt (Form E1)

Crystalline Compound A Malonate Salt (Form E1) was prepared by reactive crystallization at room temperature with malonic acid in methyl isobutyl ketone with a 1:1 molar ratio of Compound A and counterion. The resulting solids were identified as crystalline Form E1 by XRPD as shown in Table 19 and FIG. 36.

TABLE 19
Form E1 XRPD Data

| Pos. [°2θ] | FWHM [°2θ] | d-spacing [Å] | Rel. Int. [%] |
|---|---|---|---|
| 5.5 | 0.06 | 16.23 | 24.31 |
| 6.8 | 0.08 | 12.97 | 26.18 |
| 8.3 | 0.11 | 10.63 | 22.03 |
| 10.3 | 0.13 | 8.59 | 12.67 |
| 10.9 | 0.15 | 8.10 | 5.52 |
| 12.6 | 0.10 | 7.03 | 35.75 |
| 15.0 | 0.11 | 5.90 | 5.55 |
| 16.6 | 0.14 | 5.35 | 100.00 |
| 17.2 | 0.15 | 5.16 | 18.96 |
| 18.0 | 0.13 | 4.94 | 4.22 |
| 18.5 | 0.10 | 4.79 | 7.58 |
| 19.4 | 0.12 | 4.57 | 21.02 |
| 20.4 | 0.17 | 4.35 | 49.14 |
| 21.0 | 0.19 | 4.22 | 17.29 |
| 22.0 | 0.14 | 4.04 | 34.15 |
| 22.9 | 0.09 | 3.88 | 18.48 |
| 24.6 | 0.15 | 3.62 | 7.24 |
| 25.3 | 0.15 | 3.52 | 8.44 |
| 25.7 | 0.17 | 3.47 | 6.83 |
| 26.2 | 0.24 | 3.40 | 17.24 |
| 26.9 | 0.11 | 3.32 | 8.62 |
| 27.8 | 0.15 | 3.21 | 8.90 |
| 28.0 | 0.19 | 3.19 | 11.92 |
| 29.8 | 0.19 | 3.00 | 7.31 |
| 30.3 | 0.22 | 2.95 | 3.58 |
| 31.5 | 0.11 | 2.84 | 6.52 |
| 31.9 | 0.22 | 2.81 | 3.08 |
| 32.6 | 0.26 | 2.75 | 0.98 |
| 33.2 | 0.22 | 2.70 | 2.28 |
| 35.7 | 0.35 | 2.51 | 1.75 |
| 37.2 | 0.69 | 2.42 | 1.08 |
| 38.2 | 0.22 | 2.36 | 1.82 |

### Example 15: Crystalline Compound A Naphthalene-2-Sulfonate Salt (Form F1)

Crystalline Compound A Naphthalene-2-Sulfonate Salt Form F1 was prepared by reactive crystallization at room temperature with naphthalene-2-sulfonic acid in acetone/heptane (1:1) with a 1:1 molar ratio of Compound A and counterion. Form F1 was also prepared by reactive crystallization at room temperature in dioxane, and methyl isobutyl ketone with a 1:1 molar ratio of Compound A and counterion. The resulting solids were identified by XRPD as crystalline Form F1 as shown in Table 20 and FIG. 40.

TABLE 20
Form F1 XRPD Data

| Pos. [°2θ] | FWHM [°2θ] | d-spacing [Å] | Rel. Int. [%] |
|---|---|---|---|
| 4.7 | 0.08 | 19.01 | 100.00 |
| 9.8 | 0.26 | 9.02 | 1.75 |
| 13.8 | 0.09 | 6.44 | 5.91 |
| 14.0 | 0.08 | 6.31 | 6.82 |
| 14.7 | 0.09 | 6.03 | 16.73 |
| 14.9 | 0.06 | 5.94 | 34.52 |
| 17.0 | 0.05 | 5.20 | 25.06 |
| 18.0 | 0.13 | 4.94 | 8.86 |
| 18.7 | 0.17 | 4.74 | 2.71 |
| 19.6 | 0.09 | 4.52 | 22.05 |
| 22.1 | 0.13 | 4.02 | 14.24 |
| 24.0 | 0.17 | 3.71 | 2.92 |
| 26.5 | 0.15 | 3.36 | 3.47 |
| 27.1 | 0.13 | 3.29 | 4.57 |
| 28.8 | 0.17 | 3.10 | 2.09 |
| 29.8 | 0.52 | 3.00 | 0.72 |

### Example 16: Crystalline Compound A Naphthalene-2-Sulfonate Salt (Form F2)

Crystalline Compound A Naphthalene-2-Sulfonate Salt Form F2 was formed by reactive crystallization at room temperature with naphthalene-2-sulfonic acid in ethanol with a 1:1 molar ratio of Compound A and counterion. The resulting solids were identified using XRPD as crystalline Form F2, as shown in Table 21 and FIG. 43.

TABLE 21
Form F2 XRPD Data

| Pos. [°2θ] | FWHM [°2θ] | d-spacing [Å] | Rel. Int. [%] |
|---|---|---|---|
| 5.8 | 0.06 | 15.18 | 100.00 |
| 6.9 | 0.15 | 12.77 | 1.29 |
| 9.4 | 0.04 | 9.45 | 6.81 |
| 11.6 | 0.08 | 7.60 | 16.58 |
| 14.0 | 0.06 | 6.33 | 13.54 |
| 15.2 | 0.15 | 5.85 | 0.99 |
| 16.8 | 0.09 | 5.29 | 6.21 |
| 17.5 | 0.09 | 5.07 | 14.81 |
| 18.8 | 0.06 | 4.73 | 5.08 |
| 19.7 | 0.09 | 4.50 | 12.36 |
| 20.0 | 0.04 | 4.44 | 5.22 |
| 20.9 | 0.10 | 4.26 | 5.74 |
| 21.9 | 0.10 | 4.06 | 1.22 |
| 22.8 | 0.10 | 3.90 | 2.17 |
| 23.8 | 0.10 | 3.74 | 2.85 |
| 24.3 | 0.13 | 3.67 | 1.84 |
| 25.2 | 0.10 | 3.53 | 3.98 |
| 25.8 | 0.06 | 3.46 | 4.82 |
| 27.4 | 0.15 | 3.26 | 1.81 |
| 28.3 | 0.18 | 3.15 | 1.44 |
| 31.8 | 0.41 | 2.82 | 0.58 |

### Example 17: Crystalline Compound A Naphthalene-2-Sulfonate Salt (Form F3)

Crystalline Compound A Naphthalene-2-Sulfonate Salt Form F3 was formed by reactive crystallization at room temperature with naphthalene-2-sulfonic acid in dichloromethane with a 1:1 molar ratio of Compound A and counterion. Form F3 was also formed by reactive crystallization with naphthalene-2-sulfonic acid at 5 C in acetonitrile/water (1:1) with a 1:1 molar ratio of Compound A to counterion. The resulting solids were identified using XRPD as crystalline Form F3, as shown in Table 22 and FIG. 44.

TABLE 22
Form F3 XRPD Data

| Pos. [°2θ] | FWHM [°2θ] | d-spacing [Å] | Rel. Int. [%] |
|---|---|---|---|
| 3.8 | 0.10 | 23.02 | 97.70 |
| 7.6 | 0.10 | 11.57 | 8.95 |
| 8.9 | 0.20 | 9.90 | 1.70 |
| 9.7 | 0.13 | 9.10 | 6.32 |
| 11.5 | 0.10 | 7.72 | 76.54 |
| 15.3 | 0.13 | 5.79 | 100.00 |
| 15.7 | 0.13 | 5.66 | 4.58 |
| 16.4 | 0.13 | 5.40 | 2.88 |
| 17.0 | 0.20 | 5.22 | 1.47 |
| 17.4 | 0.15 | 5.09 | 2.57 |
| 17.9 | 0.15 | 4.95 | 2.16 |
| 18.4 | 0.15 | 4.82 | 1.26 |
| 18.9 | 0.18 | 4.69 | 1.42 |
| 19.5 | 0.10 | 4.55 | 2.62 |
| 20.2 | 0.26 | 4.40 | 3.60 |
| 21.2 | 0.20 | 4.19 | 1.28 |
| 23.0 | 0.13 | 3.87 | 4.31 |
| 25.6 | 0.15 | 3.49 | 1.22 |
| 26.4 | 0.13 | 3.38 | 3.06 |
| 26.7 | 0.15 | 3.34 | 1.80 |
| 30.0 | 0.20 | 2.98 | 1.53 |

### Example 18: Crystalline Compound A Methane Sulfonate Salt (Form G1)

Crystalline Compound A Methane Sulfonate Salt Form G1 was prepared by reactive crystallization at room temperature with methane sulfonic acid in methyl isobutyl ketone with a 1:1 molar ratio of Compound A and counterion. The resulting solids were identified using XRPD as crystalline Form G1, as shown in Table 23 and FIG. 46.

TABLE 23
Form G1 XRPD Data

| Pos. [°2θ] | FWHM [°2θ] | d-spacing [Å] | Rel. Int. [%] |
|---|---|---|---|
| 5.1 | 0.10 | 17.45 | 42.05 |
| 6.5 | 0.08 | 13.68 | 100.00 |
| 6.9 | 0.08 | 12.82 | 33.49 |
| 9.3 | 0.04 | 9.49 | 28.01 |
| 12.3 | 0.20 | 7.20 | 6.51 |
| 13.6 | 0.10 | 6.54 | 74.01 |
| 13.8 | 0.06 | 6.43 | 59.80 |
| 16.7 | 0.10 | 5.31 | 9.27 |
| 18.5 | 0.18 | 4.79 | 36.21 |
| 19.5 | 0.10 | 4.56 | 92.90 |
| 20.4 | 0.26 | 4.35 | 8.10 |
| 20.8 | 0.15 | 4.28 | 10.74 |
| 21.5 | 0.13 | 4.13 | 21.02 |
| 22.4 | 0.15 | 3.98 | 4.15 |
| 25.9 | 0.20 | 3.44 | 9.87 |
| 28.0 | 0.26 | 3.19 | 4.80 |

### Example 19: Crystalline Compound A Methane Sulfonate Salt (Form G2)

Crystalline Compound A Methane Sulfonate Salt Form G2 was prepared by reactive crystallization at room temperature with methane sulfonic acid in ethanol with a 1:1 molar ratio of Compound A and counterion. Form G2 was also formed by reactive crystallization at room temperature in dichloromethane with a 1:1 molar ratio of Compound A and counterion. The resulting solids were identified using XRPD as crystalline Form G2, as shown in Table 24 and FIG. 47.

TABLE 24
Form G21 XRPD Data

| Pos. [°2θ] | FWHM [°2θ] | d-spacing [Å] | Rel. Int. [%] |
|---|---|---|---|
| 5.6 | 0.06 | 15.70 | 22.57 |
| 8.1 | 0.10 | 10.87 | 4.54 |
| 11.2 | 0.15 | 7.87 | 1.62 |
| 12.5 | 0.06 | 7.07 | 10.99 |
| 13.1 | 0.10 | 6.74 | 100.00 |
| 13.3 | 0.09 | 6.64 | 43.65 |
| 16.3 | 0.12 | 5.45 | 21.52 |
| 16.9 | 0.10 | 5.26 | 8.59 |
| 17.2 | 0.10 | 5.15 | 4.49 |
| 18.3 | 0.13 | 4.85 | 19.92 |
| 19.2 | 0.15 | 4.63 | 1.75 |
| 19.8 | 0.12 | 4.50 | 12.26 |
| 20.4 | 0.15 | 4.36 | 5.39 |
| 20.8 | 0.15 | 4.27 | 1.84 |
| 21.3 | 0.08 | 4.17 | 8.66 |
| 21.5 | 0.06 | 4.13 | 9.74 |
| 22.5 | 0.13 | 3.94 | 3.52 |
| 23.1 | 0.13 | 3.85 | 7.45 |
| 23.6 | 0.14 | 3.77 | 9.38 |
| 24.6 | 0.18 | 3.61 | 1.95 |
| 25.2 | 0.20 | 3.54 | 1.71 |
| 26.1 | 0.13 | 3.42 | 2.62 |
| 27.0 | 0.18 | 3.30 | 3.23 |
| 27.6 | 0.13 | 3.23 | 2.14 |
| 28.3 | 0.15 | 3.16 | 0.99 |
| 29.4 | 0.15 | 3.04 | 0.92 |
| 30.3 | 0.10 | 2.95 | 3.07 |
| 34.4 | 0.20 | 2.61 | 1.04 |
| 35.0 | 0.15 | 2.56 | 1.46 |
| 36.7 | 0.15 | 2.45 | 1.00 |
| 37.8 | 0.51 | 2.38 | 0.72 |

### Example 20: Crystalline Compound A Oxalate Salt (Form H2)

Crystalline Compound A Oxalate Salt Form H2 was prepared by reactive crystallization at room temperature with oxalic acid in dichloromethane with a 1:1 molar ratio of Compound A and counterion. Form H2 was also formed by reactive crystallization at 5° C. in acetonitrile/water (1:1) with a 1:1 molar ratio of Compound A and counterion. The resulting solids were identified using XRPD as crystalline Form H2, as shown in Table 25 and FIG. 50.

TABLE 25
Form H2 XRPD Data

| Pos. [°2θ] | FWHM [°2θ] | d-spacing [Å] | Rel. Int. [%] |
|---|---|---|---|
| 6.0 | 0.08 | 14.65 | 3.73 |
| 6.9 | 0.10 | 12.79 | 20.72 |
| 8.7 | 0.10 | 10.21 | 34.23 |
| 11.0 | 0.08 | 8.06 | 10.35 |
| 12.4 | 0.10 | 7.12 | 8.24 |
| 12.8 | 0.10 | 6.92 | 2.98 |
| 13.6 | 0.08 | 6.52 | 29.18 |
| 15.4 | 0.10 | 5.76 | 2.00 |
| 16.3 | 0.08 | 5.42 | 6.62 |
| 17.4 | 0.13 | 5.11 | 100.00 |
| 18.1 | 0.10 | 4.89 | 2.99 |
| 19.6 | 0.10 | 4.53 | 10.83 |
| 20.5 | 0.08 | 4.34 | 7.50 |
| 20.8 | 0.08 | 4.27 | 13.66 |
| 21.0 | 0.08 | 4.24 | 12.74 |
| 22.0 | 0.13 | 4.03 | 16.16 |
| 22.3 | 0.10 | 3.99 | 6.59 |
| 22.9 | 0.08 | 3.89 | 1.70 |
| 23.6 | 0.15 | 3.77 | 0.84 |
| 24.3 | 0.10 | 3.67 | 2.87 |
| 24.6 | 0.13 | 3.61 | 38.48 |
| 25.0 | 0.08 | 3.56 | 7.68 |
| 25.3 | 0.10 | 3.52 | 10.60 |
| 25.7 | 0.10 | 3.46 | 5.04 |
| 26.2 | 0.13 | 3.41 | 3.01 |
| 26.6 | 0.10 | 3.35 | 5.84 |
| 27.3 | 0.10 | 3.26 | 16.32 |
| 27.8 | 0.15 | 3.20 | 0.99 |
| 28.4 | 0.10 | 3.14 | 1.54 |
| 29.0 | 0.10 | 3.08 | 6.39 |
| 29.3 | 0.10 | 3.05 | 4.21 |
| 29.8 | 0.20 | 2.99 | 1.00 |
| 30.7 | 0.13 | 2.91 | 3.30 |
| 31.1 | 0.15 | 2.87 | 0.61 |
| 32.7 | 0.10 | 2.74 | 1.84 |
| 33.0 | 0.13 | 2.72 | 4.44 |
| 34.2 | 0.15 | 2.62 | 0.45 |
| 35.1 | 0.10 | 2.56 | 1.73 |
| 35.9 | 0.15 | 2.50 | 2.84 |
| 36.8 | 0.08 | 2.44 | 1.32 |
| 37.5 | 0.10 | 2.40 | 1.31 |
| 38.0 | 0.15 | 2.37 | 0.61 |
| 38.7 | 0.20 | 2.33 | 0.87 |

### Example 21: Crystalline Compound A Tartrate Salt (Form I1)

Crystalline Compound A Tartrate Salt Form I1 was prepared by reactive crystallization at room temperature with tartaric acid in dioxane with a 1:1 molar ratio of Compound A and counterion. The resulting solids were identified as crystalline Form I1 using XRPD, as shown in Table 26 and FIG. 52.

TABLE 26
Form I1 XRPD Data

| Pos. [°2θ] | FWHM [°2θ] | d-spacing [Å] | Rel. Int. [%] |
|---|---|---|---|
| 3.4 | 0.13 | 25.71 | 100.00 |
| 6.8 | 0.09 | 13.10 | 10.83 |
| 7.4 | 0.15 | 11.98 | 15.51 |
| 8.4 | 0.13 | 10.59 | 13.24 |
| 9.0 | 0.13 | 9.87 | 9.79 |
| 10.2 | 0.13 | 8.70 | 10.45 |
| 10.6 | 0.13 | 8.37 | 11.20 |
| 11.8 | 0.11 | 7.49 | 13.34 |
| 12.3 | 0.13 | 7.22 | 18.86 |
| 13.3 | 0.15 | 6.66 | 26.15 |
| 13.5 | 0.09 | 6.54 | 31.24 |
| 14.3 | 0.13 | 6.18 | 45.03 |
| 14.7 | 0.13 | 6.01 | 65.21 |
| 15.1 | 0.13 | 5.86 | 42.64 |
| 15.6 | 0.17 | 5.69 | 72.60 |
| 16.9 | 0.30 | 5.25 | 22.41 |
| 18.0 | 0.17 | 4.92 | 64.99 |
| 18.7 | 0.15 | 4.74 | 50.21 |
| 19.4 | 0.11 | 4.58 | 54.01 |
| 19.7 | 0.09 | 4.50 | 44.78 |
| 20.6 | 0.15 | 4.31 | 36.63 |
| 21.2 | 0.15 | 4.20 | 24.64 |
| 22.1 | 0.13 | 4.02 | 17.08 |
| 23.1 | 0.22 | 3.85 | 26.15 |
| 23.5 | 0.26 | 3.79 | 35.04 |
| 24.3 | 0.11 | 3.67 | 57.51 |
| 24.9 | 0.17 | 3.58 | 25.56 |
| 26.7 | 0.17 | 3.34 | 39.63 |
| 27.2 | 0.15 | 3.28 | 23.96 |
| 30.7 | 0.52 | 2.91 | 2.55 |

### Example 22: Crystalline Compound A Ethane Sulfonate Salt (Form J1)

Crystalline Compound A Ethane Sulfonate Salt Form J1 was prepared by reactive crystallization at room temperature with ethane sulfonic acid in ethanol with a 1:1 molar ratio of Compound A and counterion. The resulting solids were identified as crystalline Form J1 using XRPD, as shown in Table 27 and FIG. 54.

TABLE 27
Form J1 XRPD Data

| Pos. [°2θ] | FWHM [°2θ] | d-spacing [Å] | Rel. Int. [%] |
|---|---|---|---|
| 5.6 | 0.06 | 15.76 | 100.00 |
| 6.6 | 0.08 | 13.49 | 12.17 |
| 7.9 | 0.06 | 11.26 | 39.97 |
| 8.4 | 0.10 | 10.53 | 6.51 |
| 9.8 | 0.10 | 9.03 | 6.04 |
| 11.1 | 0.10 | 7.94 | 13.43 |
| 12.1 | 0.08 | 7.32 | 5.99 |
| 13.1 | 0.06 | 6.77 | 22.29 |
| 13.6 | 0.10 | 6.53 | 33.95 |
| 14.4 | 0.08 | 6.14 | 4.79 |
| 15.7 | 0.09 | 5.64 | 79.52 |
| 16.1 | 0.08 | 5.50 | 4.72 |
| 16.8 | 0.10 | 5.27 | 13.35 |
| 17.9 | 0.12 | 4.95 | 25.23 |
| 18.7 | 0.09 | 4.74 | 24.21 |
| 19.7 | 0.10 | 4.52 | 4.65 |
| 20.4 | 0.10 | 4.35 | 20.73 |
| 20.7 | 0.10 | 4.29 | 4.83 |
| 21.7 | 0.08 | 4.09 | 2.91 |
| 22.5 | 0.06 | 3.96 | 9.13 |
| 23.3 | 0.10 | 3.82 | 2.82 |
| 24.1 | 0.08 | 3.69 | 1.98 |
| 25.0 | 0.18 | 3.56 | 2.14 |
| 26.3 | 0.08 | 3.39 | 5.96 |
| 26.6 | 0.10 | 3.35 | 3.20 |
| 27.3 | 0.10 | 3.27 | 1.94 |
| 28.1 | 0.13 | 3.18 | 1.59 |
| 29.4 | 0.15 | 3.04 | 2.29 |
| 30.9 | 0.15 | 2.90 | 0.85 |
| 31.9 | 0.20 | 2.80 | 0.94 |
| 34.0 | 0.13 | 2.64 | 1.57 |
| 35.4 | 0.20 | 2.53 | 0.98 |
| 36.6 | 0.08 | 2.46 | 1.37 |
| 39.4 | 0.20 | 2.29 | 0.62 |

### Example 23: Crystalline Compound A Ethane Sulfonate Salt (Form J2)

Crystalline Compound A Ethane Sulfonate Salt Form J1 was prepared by reactive crystallization at room temperature with ethane sulfonic acid in 1:1 acetone/heptane with a 1:1 molar ratio of Compound A to counterion. Form J1 was also formed by reactive crystallization with ethane sulfonic acid at room temperature in methyl isobutyl ketone with a 1:1 molar ratio of Compound A and counterion. The resulting solids were identified as crystalline Form J1 using XRPD, as shown in Table 28 and FIG. 56.

TABLE 28
Form J2 XRPD Data

| Pos. [°2θ] | FWHM [°2θ] | d-spacing [Å] | Rel. Int. [%] |
|---|---|---|---|
| 4.0 | 0.11 | 22.20 | 89.82 |
| 6.3 | 0.10 | 13.96 | 98.62 |
| 6.9 | 0.13 | 12.76 | 32.76 |
| 7.7 | 0.06 | 11.48 | 90.82 |
| 7.9 | 0.05 | 11.22 | 79.10 |
| 8.9 | 0.17 | 9.95 | 8.96 |
| 10.3 | 0.17 | 8.60 | 9.96 |
| 11.9 | 0.13 | 7.46 | 11.55 |
| 12.3 | 0.13 | 7.20 | 22.75 |
| 12.7 | 0.09 | 7.00 | 21.23 |
| 13.9 | 0.26 | 6.40 | 16.24 |
| 14.5 | 0.13 | 6.10 | 29.56 |
| 15.8 | 0.08 | 5.60 | 98.35 |
| 16.8 | 0.19 | 5.29 | 64.02 |
| 17.6 | 0.13 | 5.04 | 34.17 |
| 17.9 | 0.13 | 4.97 | 29.86 |
| 18.4 | 0.11 | 4.83 | 65.81 |
| 18.6 | 0.09 | 4.77 | 65.63 |
| 19.0 | 0.11 | 4.67 | 40.20 |
| 19.5 | 0.09 | 4.55 | 50.68 |
| 20.0 | 0.11 | 4.43 | 72.07 |
| 20.9 | 0.15 | 4.26 | 100.00 |
| 21.6 | 0.13 | 4.12 | 42.36 |
| 22.2 | 0.13 | 4.01 | 25.65 |
| 23.3 | 0.15 | 3.83 | 34.86 |
| 23.8 | 0.11 | 3.74 | 33.52 |
| 24.1 | 0.11 | 3.69 | 44.14 |
| 24.7 | 0.15 | 3.60 | 39.88 |
| 25.9 | 0.17 | 3.45 | 24.50 |
| 26.1 | 0.15 | 3.41 | 32.85 |
| 27.0 | 0.17 | 3.30 | 18.75 |
| 27.8 | 0.15 | 3.21 | 25.66 |
| 28.4 | 0.17 | 3.14 | 28.91 |
| 29.6 | 0.35 | 3.02 | 9.06 |
| 31.9 | 0.17 | 2.81 | 8.21 |
| 35.9 | 0.52 | 2.50 | 2.20 |

### Example 24: Crystalline Compound A N-cyclohexylsulfamate Salt (Form K1)

Crystalline Compound A N-cyclohexylsulfamate Salt Form K1 was prepared reactive crystallization at room temperature with cyclamic acid in dioxane at 1:1 molar ratio of Compound A and counterion. Form K1 was also formed by reactive crystallization at room temperature with cyclamic acid in ethanol, dichloromethane, and acetone/heptane (1:1) with a 1:1 molar ratio of Compound A and counterion. Form K1 was also formed by reactive crystallization with cyclamic acid at 5° C. in acetonitrile/water (1:1) with a 1:1 molar ratio of Compound A and counterion. The resulting solids were identified as crystalline Form K1 using XRPD, as shown in Table 29 and FIG. 58.

TABLE 29
Form K1 XRPD Data

| Pos. [°2θ] | FWHM [°2θ] | d-spacing [Å] | Rel. Int. [%] |
|---|---|---|---|
| 5.8 | 0.06 | 15.29 | 100.00 |
| 8.3 | 0.08 | 10.62 | 1.05 |
| 9.3 | 0.09 | 9.53 | 1.07 |
| 11.6 | 0.06 | 7.64 | 3.88 |
| 14.0 | 0.09 | 6.35 | 5.91 |
| 15.6 | 0.08 | 5.68 | 6.17 |
| 16.7 | 0.10 | 5.31 | 15.67 |
| 17.4 | 0.13 | 5.09 | 0.45 |
| 18.6 | 0.09 | 4.76 | 1.15 |
| 19.9 | 0.09 | 4.46 | 1.68 |
| 20.8 | 0.09 | 4.27 | 1.65 |
| 23.3 | 0.11 | 3.82 | 1.56 |
| 23.6 | 0.11 | 3.76 | 1.87 |
| 24.4 | 0.11 | 3.65 | 0.84 |
| 25.6 | 0.11 | 3.48 | 2.31 |
| 28.1 | 0.13 | 3.17 | 3.92 |
| 33.1 | 0.26 | 2.70 | 0.34 |
| 37.5 | 0.26 | 2.40 | 0.27 |

### Example 25: Crystalline Compound A Maleate Salt (Form L1)

Crystalline Compound A Maleate Salt Form L1 was prepared by reactive crystallization at room temperature with maleic acid in ethanol with a 1:1 molar ratio of Compound A to counterion. Form L1 was also formed by reactive crystallization at room temperature with maleic acid in methyl isobutyl ketone, dichloromethane, dioxane, and acetone/heptane (1:1) with a 1:1 molar ratio of API to counterion. Form L1 was also observed by reactive crystallization at 5° C. with maleic acid in acetonitrile/water (1:1) with a 1:1 molar ratio of Compound A and counterion. The resulting solids were identified as crystalline Form L1 using XRPD, as shown in Table 30 and FIG. 60.

TABLE 30
Form L1 XRPD Data

| Pos. [°2θ] | FWHM [°2θ] | d-spacing [Å] | Rel. Int. [%] |
|---|---|---|---|
| 5.7 | 0.18 | 15.64 | 100.00 |
| 7.8 | 0.26 | 11.31 | 3.50 |
| 8.8 | 0.15 | 10.06 | 3.51 |
| 9.5 | 0.31 | 9.29 | 2.62 |
| 11.3 | 0.23 | 7.83 | 9.40 |
| 13.7 | 0.31 | 6.45 | 10.49 |
| 14.8 | 0.31 | 5.97 | 5.26 |
| 16.1 | 0.20 | 5.51 | 17.08 |
| 17.0 | 0.23 | 5.23 | 29.01 |
| 17.5 | 0.20 | 5.06 | 11.16 |
| 18.4 | 0.13 | 4.83 | 13.73 |
| 20.0 | 0.41 | 4.44 | 5.70 |
| 21.1 | 0.20 | 4.21 | 6.22 |
| 21.9 | 0.20 | 4.05 | 1.54 |
| 22.6 | 0.20 | 3.93 | 4.37 |
| 23.5 | 0.20 | 3.79 | 2.77 |
| 24.4 | 0.46 | 3.64 | 6.80 |
| 25.6 | 0.20 | 3.49 | 21.09 |
| 26.1 | 0.18 | 3.42 | 15.04 |
| 28.0 | 0.20 | 3.19 | 7.61 |
| 28.7 | 0.20 | 3.11 | 3.61 |
| 31.4 | 0.82 | 2.85 | 0.87 |

### Example 26: Crystalline Compound A Phosphate Salt (Form M1)

Crystalline Compound A Phosphate Salt Form M1 was prepared by reactive crystallization at room temperature with phosphoric acid in ethanol with a 1:1 molar ratio of Compound A and counterion. The resulting solids were identified as crystalline Form M1 using XRPD, as shown in Table 30 and FIG. 62.

TABLE 31
Form M1 XRPD Data

| Pos. [°2θ] | FWHM [°2θ] | d-spacing [Å] | Rel. Int. [%] |
|---|---|---|---|
| 5.4 | 0.16 | 16.41 | 100.00 |
| 8.1 | 0.15 | 10.91 | 2.86 |
| 10.8 | 0.09 | 8.17 | 10.24 |
| 12.3 | 0.13 | 7.19 | 7.15 |
| 13.2 | 0.13 | 6.73 | 1.34 |
| 13.6 | 0.13 | 6.53 | 3.14 |
| 14.5 | 0.17 | 6.11 | 0.94 |
| 16.2 | 0.11 | 5.46 | 67.09 |
| 16.3 | 0.08 | 5.45 | 58.89 |
| 19.0 | 0.12 | 4.67 | 6.46 |
| 20.3 | 0.15 | 4.37 | 10.89 |
| 21.2 | 0.16 | 4.19 | 3.55 |
| 21.8 | 0.18 | 4.08 | 9.39 |
| 22.2 | 0.16 | 3.99 | 5.40 |
| 22.5 | 0.09 | 3.94 | 12.62 |
| 23.1 | 0.13 | 3.85 | 2.08 |
| 24.0 | 0.16 | 3.70 | 5.99 |
| 24.5 | 0.32 | 3.63 | 5.19 |
| 24.9 | 0.13 | 3.58 | 2.99 |
| 25.4 | 0.26 | 3.50 | 1.41 |
| 26.6 | 0.12 | 3.35 | 4.92 |
| 27.4 | 0.18 | 3.26 | 2.79 |
| 28.2 | 0.13 | 3.17 | 2.25 |
| 28.8 | 0.16 | 3.09 | 0.85 |
| 29.9 | 0.16 | 2.99 | 3.04 |
| 30.9 | 0.42 | 2.90 | 0.48 |
| 31.9 | 0.26 | 2.80 | 0.75 |
| 32.9 | 0.18 | 2.72 | 6.11 |
| 34.0 | 0.21 | 2.64 | 0.73 |
| 35.1 | 0.16 | 2.55 | 1.45 |
| 38.4 | 0.26 | 2.34 | 0.70 |

### Example 27: Crystalline Compound A Phosphate Salt (Form M2)

Crystalline Compound A Phosphate Salt Form M2 was prepared by reactive crystallization at room temperature with phosphoric acid in dichloromethane with a 1:1 molar ratio of Compound A to counterion. The resulting solids were identified as crystalline Form M2 using XRPD, as shown in Table 31 and FIG. 65.

TABLE 31
Form M2 XRPD Data

| Pos. [°2θ] | FWHM [°2θ] | d-spacing [Å] | Rel. Int. [%] |
|---|---|---|---|
| 3.6 | 0.26 | 24.36 | 11.41 |
| 7.1 | 0.10 | 12.46 | 52.02 |
| 7.4 | 0.13 | 11.88 | 13.10 |
| 8.2 | 0.08 | 10.77 | 8.25 |
| 8.9 | 0.13 | 9.90 | 23.87 |
| 10.6 | 0.10 | 8.32 | 35.74 |
| 10.9 | 0.10 | 8.14 | 39.10 |
| 13.4 | 0.10 | 6.59 | 31.94 |
| 14.2 | 0.10 | 6.24 | 76.68 |
| 14.9 | 0.13 | 5.96 | 51.94 |
| 16.4 | 0.13 | 5.40 | 20.90 |
| 16.8 | 0.10 | 5.27 | 48.68 |
| 17.8 | 0.13 | 4.99 | 100.00 |
| 19.6 | 0.13 | 4.54 | 55.78 |
| 21.0 | 0.15 | 4.24 | 11.45 |
| 21.4 | 0.10 | 4.16 | 43.71 |
| 21.8 | 0.20 | 4.08 | 18.53 |
| 22.0 | 0.10 | 4.03 | 9.73 |
| 22.4 | 0.10 | 3.97 | 8.17 |
| 23.0 | 0.15 | 3.87 | 13.25 |
| 23.4 | 0.15 | 3.80 | 2.50 |
| 23.9 | 0.13 | 3.73 | 8.46 |
| 24.3 | 0.15 | 3.66 | 4.02 |
| 25.0 | 0.10 | 3.56 | 16.93 |
| 25.4 | 0.18 | 3.51 | 17.36 |
| 25.7 | 0.15 | 3.47 | 6.30 |
| 26.3 | 0.15 | 3.39 | 10.32 |
| 26.9 | 0.20 | 3.31 | 15.49 |
| 27.5 | 0.15 | 3.24 | 4.55 |
| 28.1 | 0.15 | 3.18 | 2.22 |
| 28.6 | 0.10 | 3.12 | 6.75 |
| 29.9 | 0.31 | 2.99 | 1.68 |
| 31.1 | 0.31 | 2.88 | 3.70 |
| 32.3 | 0.15 | 2.77 | 2.81 |
| 32.9 | 0.41 | 2.72 | 2.98 |
| 38.0 | 0.26 | 2.37 | 2.69 |

### Example 28: Crystalline Compound A Phosphate Salt (Form M3)

Crystalline Compound A Phosphate Salt Form M3 was prepared by reactive crystallization at 5° C. with phosphoric acid in dioxane with a 1:1 molar ratio of Compound A and counterion. The resulting solids were identified as crystalline Form M3 using XRPD, as shown in Table 32 and FIG. 67.

TABLE 32
Form M3 XRPD Data

| Pos. [°2θ] | FWHM [°2θ] | d-spacing [Å] | Rel. Int. [%] |
|---|---|---|---|
| 3.9 | 0.04 | 22.88 | 13.26 |
| 7.5 | 0.06 | 11.79 | 100.00 |
| 7.8 | 0.05 | 11.33 | 41.23 |
| 9.0 | 0.06 | 9.81 | 28.75 |
| 9.8 | 0.06 | 9.07 | 22.05 |
| 11.6 | 0.05 | 7.65 | 23.31 |
| 11.7 | 0.05 | 7.58 | 21.45 |
| 14.8 | 0.06 | 5.98 | 36.57 |
| 15.0 | 0.08 | 5.90 | 72.62 |
| 15.4 | 0.08 | 5.74 | 59.75 |
| 15.6 | 0.08 | 5.68 | 8.62 |
| 15.9 | 0.04 | 5.57 | 5.48 |
| 16.2 | 0.09 | 5.49 | 12.38 |
| 18.1 | 0.05 | 4.91 | 9.38 |
| 18.3 | 0.08 | 4.84 | 21.29 |
| 19.4 | 0.04 | 4.57 | 6.82 |
| 22.2 | 0.06 | 4.00 | 16.58 |
| 22.6 | 0.12 | 3.93 | 2.30 |
| 23.4 | 0.17 | 3.80 | 2.00 |
| 25.2 | 0.09 | 3.54 | 18.28 |
| 25.7 | 0.06 | 3.47 | 3.25 |
| 26.9 | 0.06 | 3.32 | 2.78 |
| 27.2 | 0.07 | 3.28 | 13.31 |
| 27.2 | 0.04 | 3.28 | 11.19 |
| 28.7 | 0.07 | 3.11 | 3.01 |
| 29.7 | 0.26 | 3.00 | 1.38 |
| 30.7 | 0.13 | 2.91 | 1.46 |
| 32.3 | 0.21 | 2.77 | 0.54 |
| 34.6 | 0.08 | 2.59 | 1.27 |
| 35.0 | 0.84 | 2.56 | 0.20 |
| 35.5 | 0.18 | 2.53 | 1.16 |
| 36.6 | 0.21 | 2.45 | 0.55 |
| 37.2 | 0.21 | 2.42 | 0.49 |
| 39.1 | 0.21 | 2.30 | 1.71 |

### Example 29: Crystalline Compound A Salicylic Acid Cocrystal (Form CC-1A)

Crystalline Compound A Salicylic Acid Cocrystal Form CC-1A was prepared by a reactive crystallization with salicylic acid in ethanol at room temperature with a 1:1 molar ratio of Compound A and salicylic acid coformer. The resulting solids were identified as crystalline Form CC-1A using XRPD, as shown in Table 33 and FIG. 69.

TABLE 33
Form CC-1A XRPD Data

| Pos. [°2θ] | FWHM [°2θ] | d-spacing [Å] | Rel. Int. [%] |
|---|---|---|---|
| 5.9 | 0.05 | 15.07 | 79.92 |
| 6.0 | 0.06 | 14.67 | 39.56 |
| 8.3 | 0.15 | 10.68 | 27.52 |
| 9.3 | 0.08 | 9.52 | 100.00 |
| 9.7 | 0.08 | 9.08 | 77.85 |
| 13.1 | 0.10 | 6.75 | 33.45 |
| 13.9 | 0.06 | 6.38 | 33.86 |
| 15.0 | 0.15 | 5.92 | 5.34 |
| 16.6 | 0.31 | 5.36 | 4.22 |
| 18.6 | 0.15 | 4.76 | 6.91 |
| 19.5 | 0.15 | 4.55 | 4.73 |
| 20.8 | 0.15 | 4.27 | 4.33 |

### Example 30: Crystalline Compound A Salicylic Acid Cocrystal (Form CC-2A)

Crystalline Compound A Salicylic Acid Cocrystal Form CC-2A was prepared by reactive crystallization with salicylic acid in methyl isobutyl ketone at room temperature with a 1:1 molar ratio of Compound A and salicylic acid coformer. The resulting solids were identified as crystalline Form CC-2A using XRPD, as shown in Table 34 and FIG. 71.

TABLE 34
Form CC-2A XRPD Data

| Pos. [°2θ] | FWHM [°2θ] | d-spacing [Å] | Rel. Int. [%] |
|---|---|---|---|
| 3.2 | 0.11 | 27.64 | 12.91 |
| 8.2 | 0.11 | 10.76 | 100.00 |
| 9.2 | 0.12 | 9.59 | 69.65 |
| 11.1 | 0.10 | 7.98 | 18.72 |
| 11.8 | 0.06 | 7.52 | 8.33 |
| 14.3 | 0.26 | 6.21 | 0.59 |
| 16.0 | 0.10 | 5.53 | 22.94 |
| 16.5 | 0.06 | 5.39 | 34.81 |
| 17.2 | 0.06 | 5.16 | 19.64 |
| 18.5 | 0.10 | 4.80 | 25.10 |
| 20.2 | 0.15 | 4.40 | 5.60 |
| 21.0 | 0.17 | 4.23 | 1.41 |
| 22.9 | 0.17 | 3.89 | 1.27 |
| 23.7 | 0.35 | 3.76 | 1.75 |
| 24.6 | 0.13 | 3.62 | 8.16 |
| 24.8 | 0.11 | 3.59 | 6.73 |
| 25.9 | 0.11 | 3.44 | 7.51 |
| 27.6 | 0.13 | 3.23 | 2.64 |
| 29.6 | 0.22 | 3.02 | 0.67 |
| 31.1 | 0.26 | 2.87 | 0.48 |
| 32.7 | 0.69 | 2.74 | 0.50 |

### Example 31: Crystalline Compound A Salicylic Acid Cocrystal (Form CC-3A)

Crystalline Compound A Salicylic Acid Cocrystal Form CC-3A was prepared by reactive crystallization with salicylic acid in dichloromethane at room temperature with a 1:1 molar ratio of Compound A and salicylic acid coformer. The resulting solids were identified as crystalline Form CC-3A using XRPD, as shown in Table 35 and FIG. 73.

TABLE 35
Form CC-3A XRPD Data

| Pos. [°2θ] | FWHM [°2θ] | d-spacing [Å] | Rel. Int. [%] |
|---|---|---|---|
| 3.6 | 0.13 | 24.27 | 100.00 |
| 6.5 | 0.15 | 13.52 | 4.54 |
| 7.2 | 0.09 | 12.24 | 19.67 |
| 8.0 | 0.17 | 11.01 | 23.51 |
| 9.4 | 0.11 | 9.46 | 3.58 |
| 10.8 | 0.13 | 8.19 | 7.14 |
| 11.2 | 0.19 | 7.92 | 14.10 |
| 12.6 | 0.12 | 7.03 | 48.06 |
| 13.0 | 0.13 | 6.79 | 10.25 |
| 14.4 | 0.19 | 6.15 | 21.51 |
| 15.7 | 0.15 | 5.64 | 14.97 |
| 16.1 | 0.17 | 5.50 | 6.22 |
| 17.4 | 0.06 | 5.09 | 5.45 |
| 18.8 | 0.26 | 4.73 | 2.43 |
| 19.5 | 0.22 | 4.55 | 4.83 |
| 21.5 | 0.19 | 4.14 | 9.41 |
| 22.0 | 0.17 | 4.05 | 5.07 |
| 23.0 | 0.17 | 3.86 | 2.09 |
| 24.9 | 0.13 | 3.58 | 2.59 |
| 26.0 | 0.17 | 3.42 | 2.29 |
| 27.2 | 0.43 | 3.28 | 1.66 |
| 29.0 | 0.26 | 3.08 | 1.69 |
| 31.1 | 0.26 | 2.88 | 0.70 |
| 32.8 | 0.26 | 2.73 | 0.63 |
| 36.5 | 0.52 | 2.46 | 0.47 |

### Example 32: Crystalline Compound A Salicylic Acid Cocrystal (Form CC-4A)

Crystalline Compound A Salicylic Acid Cocrystal Form CC-4A was prepared by reactive crystallization with salicylic acid at room temperature in acetonitrile/water (1:1) or acetone/heptane (1:1) with a 1:1 molar ratio of Compound A and salicylic acid coformer. The resulting solids were identified as crystalline Form CC-4A using XRPD, as shown in Table 36 and FIG. 76.

TABLE 36
Form CC-4A XRPD Data

| Pos. [°2θ] | FWHM [°2θ] | d-spacing [Å] | Rel. Int. [%] |
|---|---|---|---|
| 3.3 | 0.13 | 26.87 | 24.83 |
| 6.4 | 0.05 | 13.76 | 28.06 |
| 9.7 | 0.05 | 9.16 | 30.11 |
| 9.9 | 0.19 | 8.90 | 69.06 |
| 10.3 | 0.22 | 8.56 | 100.00 |
| 11.0 | 0.26 | 8.08 | 4.46 |
| 11.9 | 0.22 | 7.46 | 3.34 |
| 12.8 | 0.17 | 6.93 | 30.65 |
| 13.9 | 0.17 | 6.38 | 4.77 |
| 14.3 | 0.17 | 6.19 | 5.04 |
| 15.0 | 0.15 | 5.90 | 15.57 |
| 15.6 | 0.19 | 5.68 | 22.26 |
| 16.2 | 0.10 | 5.48 | 92.12 |
| 16.3 | 0.09 | 5.43 | 67.19 |
| 17.4 | 0.13 | 5.11 | 8.83 |
| 17.6 | 0.13 | 5.03 | 13.44 |
| 18.0 | 0.15 | 4.94 | 38.30 |
| 18.5 | 0.13 | 4.79 | 4.40 |
| 19.1 | 0.17 | 4.65 | 15.69 |
| 19.9 | 0.22 | 4.46 | 49.36 |
| 20.8 | 0.22 | 4.27 | 6.29 |
| 21.4 | 0.19 | 4.16 | 23.43 |
| 21.7 | 0.15 | 4.10 | 17.76 |
| 22.8 | 0.13 | 3.91 | 13.00 |
| 23.8 | 0.26 | 3.74 | 4.30 |
| 24.6 | 0.22 | 3.62 | 16.22 |
| 24.9 | 0.17 | 3.58 | 24.37 |
| 25.3 | 0.17 | 3.52 | 39.42 |
| 25.8 | 0.17 | 3.46 | 28.48 |
| 26.3 | 0.13 | 3.39 | 44.11 |
| 27.0 | 0.08 | 3.30 | 40.56 |
| 27.8 | 0.17 | 3.22 | 15.41 |
| 28.5 | 0.39 | 3.13 | 13.60 |
| 30.1 | 0.69 | 2.97 | 5.14 |
| 33.6 | 0.17 | 2.67 | 4.12 |
| 35.0 | 0.69 | 2.56 | 3.07 |

### Example 33: Crystalline Compound A Salicylic Acid Cocrystal (Form CC-5A)

Crystalline Compound A Salicylic Acid Cocrystal Form CC-5A was prepared by slow evaporation with salicylic acid in dioxane at 25° C. with a 1:1 molar ratio Compound A and coformer. The resulting solids were identified as crystalline Form CC-5A using XRPD, as shown in Table 37 and FIG. 78.

TABLE 37
Form CC-5A XRPD Data

| Pos. [°2θ] | FWHM [°2θ] | d-spacing [Å] | Rel. Int. [%] |
|---|---|---|---|
| 4.4 | 0.13 | 20.29 | 6.16 |
| 5.3 | 0.10 | 16.70 | 100.00 |
| 8.7 | 0.13 | 10.20 | 6.44 |
| 9.2 | 0.15 | 9.62 | 14.96 |
| 10.6 | 0.10 | 8.32 | 33.07 |
| 11.0 | 0.22 | 8.03 | 14.21 |
| 11.3 | 0.13 | 7.80 | 8.04 |
| 12.2 | 0.19 | 7.24 | 14.48 |
| 12.9 | 0.11 | 6.88 | 10.10 |
| 13.2 | 0.13 | 6.69 | 8.83 |
| 14.6 | 0.17 | 6.06 | 3.99 |
| 15.5 | 0.09 | 5.73 | 18.64 |
| 15.9 | 0.11 | 5.58 | 30.28 |
| 16.5 | 0.17 | 5.38 | 7.69 |
| 17.8 | 0.16 | 4.97 | 96.20 |
| 18.3 | 0.15 | 4.85 | 32.41 |
| 19.1 | 0.11 | 4.66 | 16.89 |
| 19.8 | 0.17 | 4.48 | 9.33 |
| 20.7 | 0.17 | 4.28 | 8.67 |
| 21.3 | 0.13 | 4.18 | 9.97 |
| 22.5 | 0.26 | 3.95 | 3.36 |
| 23.4 | 0.39 | 3.81 | 5.91 |
| 24.3 | 0.26 | 3.66 | 9.19 |
| 25.7 | 0.11 | 3.47 | 23.82 |
| 26.6 | 0.13 | 3.35 | 8.27 |
| 27.2 | 0.17 | 3.28 | 8.07 |
| 27.9 | 0.26 | 3.20 | 4.32 |
| 28.7 | 0.09 | 3.11 | 15.23 |
| 30.3 | 0.13 | 2.95 | 2.74 |
| 32.6 | 0.26 | 2.75 | 2.71 |

### Example 34: Crystalline Compound A Formic Acid Cocrystal (Form CC-1B)

Crystalline Compound A Formic Acid Cocrystal Form CC-1B was prepared by slow evaporation with formic acid in dichloromethane at 25° C. with a 1:1 molar ratio of Compound A and formic acid coformer. The resulting solids were identified as crystalline Form CC-1B using XRPD, as shown in Table 38 and FIG. 80.

TABLE 38
Form CC-1B XRPD Data

| Pos. [°2θ] | FWHM [°2θ] | d-spacing [Å] | Rel. Int. [%] |
|---|---|---|---|
| 4.6 | 0.08 | 19.08 | 67.76 |
| 8.6 | 0.08 | 10.23 | 100.00 |
| 10.6 | 0.17 | 8.34 | 10.94 |
| 11.3 | 0.11 | 7.87 | 28.19 |
| 14.8 | 0.30 | 6.00 | 22.80 |
| 15.7 | 0.30 | 5.66 | 11.18 |
| 16.5 | 0.15 | 5.37 | 33.34 |
| 17.4 | 0.11 | 5.11 | 43.03 |
| 17.8 | 0.22 | 4.99 | 45.73 |
| 18.4 | 0.17 | 4.83 | 20.15 |
| 19.3 | 0.22 | 4.61 | 21.90 |
| 20.7 | 0.17 | 4.28 | 17.46 |
| 23.0 | 0.09 | 3.88 | 36.67 |
| 24.9 | 0.26 | 3.58 | 13.37 |
| 26.8 | 0.17 | 3.33 | 36.18 |
| 28.1 | 0.26 | 3.18 | 7.98 |
| 29.6 | 0.35 | 3.02 | 4.25 |
| 30.9 | 0.43 | 2.90 | 3.69 |
| 35.9 | 0.52 | 2.50 | 2.08 |

### Example 35: Crystalline Compound A Benzoic Acid Cocrystal (Form CC-1C)

Crystalline Compound A Benzoic Acid Cocrystal Form CC-1C was prepared by reactive crystallization at room temperature with benzoic acid in methyl isobutyl ketone with a 1:1 molar ratio of Compound A and benzoic acid coformer. Form CC-1C was also formed by reactive crystallization with benzoic acid in dichloromethane or acetonitrile/water (1:1) at 5° C. at a 1:1 molar ratio of Compound A and coformer. The resulting solids were identified as crystalline Form CC-1C using XRPD, as shown in Table 39 and FIG. 82.

TABLE 39
Form CC-1C XRPD Data

| Pos. [°2θ] | FWHM [°2θ] | d-spacing [Å] | Rel. Int. [%] |
|---|---|---|---|
| 3.9 | 0.05 | 22.89 | 66.09 |
| 7.7 | 0.11 | 11.47 | 9.23 |
| 8.8 | 0.13 | 10.00 | 14.46 |
| 10.4 | 0.09 | 8.52 | 22.54 |
| 10.6 | 0.05 | 8.38 | 31.65 |
| 11.6 | 0.13 | 7.65 | 100.00 |
| 12.4 | 0.10 | 7.15 | 17.27 |
| 14.2 | 0.13 | 6.24 | 70.16 |
| 14.5 | 0.11 | 6.13 | 25.21 |
| 16.1 | 0.11 | 5.52 | 77.13 |
| 17.3 | 0.22 | 5.13 | 33.97 |
| 17.7 | 0.11 | 5.00 | 12.49 |
| 18.3 | 0.08 | 4.86 | 28.29 |
| 18.9 | 0.09 | 4.69 | 6.59 |
| 19.4 | 0.13 | 4.57 | 23.87 |
| 19.8 | 0.11 | 4.48 | 53.21 |
| 20.9 | 0.13 | 4.26 | 4.22 |
| 21.7 | 0.13 | 4.09 | 15.97 |
| 22.6 | 0.11 | 3.93 | 5.12 |
| 23.2 | 0.15 | 3.83 | 9.40 |
| 24.8 | 0.26 | 3.60 | 3.60 |
| 25.4 | 0.13 | 3.50 | 2.88 |
| 25.7 | 0.11 | 3.46 | 4.40 |
| 26.3 | 0.13 | 3.39 | 9.15 |
| 26.7 | 0.11 | 3.34 | 10.02 |
| 27.2 | 0.17 | 3.28 | 6.30 |
| 28.1 | 0.26 | 3.18 | 2.29 |
| 29.5 | 0.26 | 3.03 | 1.26 |
| 30.5 | 0.17 | 2.94 | 3.53 |
| 31.2 | 0.13 | 2.87 | 1.75 |
| 32.7 | 0.26 | 2.74 | 0.89 |
| 36.4 | 0.35 | 2.47 | 1.07 |
| 37.6 | 0.13 | 2.39 | 2.19 |

### Example 36: Crystalline Compound A Isobutyric Acid Cocrystal (Form CC-1D)

Crystalline Compound A Isobutyric Acid Cocrystal Form CC-1D was prepared by reactive crystallization with isobutyric acid in acetone/heptane (1:1) or methyl isobutyl ketone at room temperature in a 1:1 molar ratio of isobutyric acid coformer with Compound A. Form CC-1D was also formed by slow evaporation at room temperature from acetonitrile/water (1:1) with a 1:1 molar ratio of coformer with Compound A. The resulting solids were identified as crystalline Form CC-1D using XRPD, as shown in Table 40 and FIG. 85.

TABLE 40
Form CC-1D XRPD Data

| Pos. [°2θ] | FWHM [°2θ] | d-spacing [Å] | Rel. Int. [%] |
|---|---|---|---|
| 5.6 | 0.06 | 15.87 | 100.00 |
| 6.1 | 0.08 | 14.46 | 75.05 |
| 6.5 | 0.08 | 13.52 | 23.35 |
| 8.0 | 0.06 | 11.10 | 43.32 |
| 8.6 | 0.09 | 10.34 | 10.61 |
| 10.7 | 0.08 | 8.27 | 19.18 |
| 11.1 | 0.08 | 7.95 | 18.65 |
| 12.2 | 0.06 | 7.25 | 12.52 |
| 13.1 | 0.06 | 6.77 | 80.17 |
| 13.8 | 0.06 | 6.43 | 3.97 |
| 14.8 | 0.13 | 5.98 | 2.36 |
| 16.0 | 0.11 | 5.55 | 49.89 |
| 17.1 | 0.10 | 5.17 | 69.87 |
| 18.2 | 0.04 | 4.87 | 19.80 |
| 19.6 | 0.06 | 4.54 | 22.68 |
| 20.1 | 0.11 | 4.43 | 6.77 |
| 22.2 | 0.17 | 4.00 | 2.86 |
| 22.8 | 0.17 | 3.89 | 3.38 |
| 23.6 | 0.11 | 3.77 | 6.68 |
| 26.3 | 0.11 | 3.39 | 6.52 |
| 33.7 | 0.26 | 2.66 | 0.82 |
| 34.8 | 0.13 | 2.58 | 1.69 |

### Example 37: Crystalline Compound A Isobutyric Acid Cocrystal (Form CC-2D)

Crystalline Compound A Isobutyric Acid Cocrystal Form CC-2D was prepared by slow evaporation at room temperature with isobutyric acid from dichloromethane in a 1:1 ratio molar ratio of Compound A and isobutyric acid coformer. The resulting solids were identified as crystalline Form CC-2D using XRPD, as shown in Table 41 and FIG. 88.

TABLE 41
Form CC-2D XRPD Data

| Pos. [°2θ] | FWHM [°2θ] | d-spacing [Å] | Rel. Int. [%] |
|---|---|---|---|
| 5.2 | 0.08 | 17.11 | 100.00 |
| 5.5 | 0.08 | 15.95 | 91.24 |
| 6.3 | 0.18 | 14.00 | 24.05 |
| 6.8 | 0.10 | 12.92 | 15.04 |
| 8.2 | 0.26 | 10.73 | 5.29 |
| 10.3 | 0.09 | 8.59 | 31.95 |
| 11.1 | 0.08 | 8.00 | 8.46 |
| 12.1 | 0.10 | 7.32 | 16.07 |
| 12.6 | 0.31 | 7.02 | 18.77 |
| 13.0 | 0.26 | 6.79 | 16.14 |
| 14.3 | 0.09 | 6.18 | 11.78 |
| 15.5 | 0.08 | 5.73 | 3.79 |
| 16.6 | 0.20 | 5.34 | 8.79 |
| 17.8 | 0.15 | 4.99 | 8.54 |
| 18.9 | 0.41 | 4.69 | 5.31 |
| 20.0 | 0.15 | 4.44 | 5.93 |
| 20.6 | 0.13 | 4.30 | 7.96 |
| 21.6 | 0.10 | 4.11 | 5.73 |
| 22.2 | 0.15 | 4.01 | 3.24 |
| 24.1 | 0.13 | 3.69 | 7.43 |
| 25.8 | 0.31 | 3.46 | 1.96 |

### Example 38: Crystalline Compound A Caprylic Acid Cocrystal (Form CC-1E)

Crystalline Compound A Caprylic Acid Cocrystal was prepared by slow evaporation at room temperature with caprylic acid in acetonitrile/water (1:1) with a 1:1 ratio of caprylic acid coformer with Compound A. The resulting solids were identified as crystalline Form CC-1E using XRPD, as shown in Table 42 and FIG. 91.

TABLE 42
Form CC-1E XRPD Data

| Pos. [°2θ] | FWHM [°2θ] | d-spacing [Å] | Rel. Int. [%] |
|---|---|---|---|
| 4.8 | 0.11 | 18.40 | 100.00 |
| 6.2 | 0.08 | 14.19 | 38.24 |
| 6.5 | 0.09 | 13.69 | 21.21 |
| 9.5 | 0.11 | 9.29 | 3.20 |
| 11.7 | 0.15 | 7.56 | 3.95 |
| 13.0 | 0.26 | 6.81 | 2.17 |
| 13.6 | 0.19 | 6.52 | 2.45 |
| 14.5 | 0.13 | 6.13 | 4.44 |
| 15.6 | 0.15 | 5.70 | 12.13 |
| 17.5 | 0.11 | 5.06 | 5.18 |
| 18.1 | 0.17 | 4.89 | 24.87 |
| 19.5 | 0.35 | 4.55 | 5.71 |
| 20.6 | 0.15 | 4.31 | 9.21 |
| 21.1 | 0.17 | 4.21 | 15.05 |
| 21.4 | 0.19 | 4.14 | 10.25 |
| 22.4 | 0.09 | 3.98 | 14.50 |
| 23.1 | 0.13 | 3.85 | 4.66 |
| 23.8 | 0.17 | 3.74 | 3.94 |
| 24.5 | 0.13 | 3.63 | 5.56 |
| 24.9 | 0.11 | 3.58 | 8.14 |
| 25.9 | 0.22 | 3.44 | 4.61 |
| 27.0 | 0.26 | 3.30 | 2.45 |
| 28.0 | 0.26 | 3.19 | 2.70 |
| 29.4 | 0.17 | 3.04 | 1.73 |
| 30.4 | 0.17 | 2.94 | 1.04 |
| 31.5 | 0.35 | 2.84 | 1.10 |
| 32.5 | 0.30 | 2.75 | 1.30 |
| 35.6 | 0.52 | 2.52 | 0.56 |
| 37.4 | 0.69 | 2.41 | 0.45 |

### Example 39: Crystalline Compound A Sorbic Acid Cocrystal (Form CC-1F)

Crystalline Compound A Sorbic Acid Cocrystal Form CC-1F was prepared by reactive crystallization with sorbic acid in acetonitrile/water (1:1) with a 1:1 ratio of Compound A to sorbic acid coformer at 5° C. The resulting solids were identified as crystalline Form CC-1F using XRPD, as shown in Table 43 and FIG. 93.

TABLE 43
Form CC-1F XRPD Data

| Pos. [°2θ] | FWHM [°2θ] | d-spacing [Å] | Rel. Int. [%] |
|---|---|---|---|
| 5.4 | 0.11 | 16.28 | 10.72 |
| 7.1 | 0.19 | 12.43 | 14.57 |
| 8.2 | 0.11 | 10.81 | 26.58 |
| 8.9 | 0.17 | 9.94 | 10.40 |
| 10.8 | 0.15 | 8.21 | 100.00 |
| 11.4 | 0.17 | 7.77 | 30.64 |
| 12.7 | 0.14 | 6.95 | 19.78 |
| 13.4 | 0.13 | 6.61 | 18.51 |
| 14.1 | 0.15 | 6.27 | 8.93 |
| 14.8 | 0.17 | 6.01 | 19.15 |
| 16.3 | 0.17 | 5.45 | 3.94 |
| 16.7 | 0.17 | 5.31 | 5.22 |
| 17.7 | 0.08 | 5.00 | 18.06 |
| 18.2 | 0.15 | 4.88 | 84.25 |
| 19.5 | 0.10 | 4.56 | 9.08 |
| 20.7 | 0.22 | 4.30 | 4.72 |
| 21.2 | 0.15 | 4.18 | 46.24 |
| 21.6 | 0.11 | 4.12 | 10.19 |
| 22.9 | 0.26 | 3.88 | 2.62 |
| 24.6 | 0.19 | 3.63 | 11.63 |
| 25.6 | 0.13 | 3.47 | 1.47 |
| 26.2 | 0.17 | 3.41 | 2.92 |
| 26.8 | 0.22 | 3.32 | 2.55 |
| 27.7 | 0.13 | 3.22 | 3.88 |
| 28.2 | 0.13 | 3.16 | 2.83 |
| 28.5 | 0.13 | 3.14 | 2.77 |
| 28.9 | 0.13 | 3.09 | 1.08 |
| 29.6 | 0.17 | 3.02 | 1.89 |
| 30.3 | 0.22 | 2.95 | 1.83 |
| 31.1 | 0.17 | 2.88 | 1.27 |
| 32.3 | 0.09 | 2.77 | 2.58 |
| 35.2 | 0.17 | 2.55 | 1.09 |
| 36.0 | 0.35 | 2.49 | 0.37 |
| 37.9 | 0.30 | 2.37 | 0.71 |

### Example 40: Crystalline Compound A Saccharin Cocrystal (Form CC-1G)

Crystalline Compound A Saccharin Cocrystal Form CC-1G was prepared by reactive crystallization at room temperature with saccharin in acetone/heptane (1:1) with a 1:1 ratio of Compound A and saccharin coformer. Form CC-1G was also formed by reactive crystallization at room temperature with saccharin in ethanol, methyl isobutyl ketone, dichloromethane, or acetonitrile/water (1:1) with a 1:1 molar ratio of Compound A and saccharin coformer. Form CC-1G was also formed by slow evaporation at room temperature with saccharine from dioxane with 1:1 molar ratio of Compound A and coformer. The resulting solids were identified as crystalline Form CC-1G using XRPD, as shown in Table 44 and FIG. 95.

TABLE 44
Form CC-1G XRPD Data

| Pos. [°2θ] | FWHM [°2θ] | d-spacing [Å] | Rel. Int. [%] |
|---|---|---|---|
| 5.1 | 0.11 | 17.44 | 100.00 |
| 9.8 | 0.13 | 8.99 | 7.02 |
| 10.1 | 0.09 | 8.74 | 7.73 |
| 11.9 | 0.13 | 7.42 | 2.27 |
| 14.2 | 0.09 | 6.25 | 4.14 |
| 14.7 | 0.13 | 6.03 | 0.97 |
| 15.2 | 0.13 | 5.81 | 3.66 |
| 16.1 | 0.06 | 5.50 | 6.24 |
| 16.5 | 0.10 | 5.37 | 7.15 |
| 17.0 | 0.17 | 5.21 | 2.21 |
| 17.9 | 0.13 | 4.97 | 0.71 |
| 18.9 | 0.13 | 4.70 | 1.64 |
| 19.7 | 0.13 | 4.50 | 0.83 |
| 20.4 | 0.13 | 4.36 | 10.55 |
| 21.2 | 0.13 | 4.20 | 1.00 |
| 21.8 | 0.13 | 4.07 | 1.66 |
| 22.5 | 0.15 | 3.95 | 2.39 |
| 23.8 | 0.13 | 3.74 | 1.89 |
| 24.7 | 0.13 | 3.61 | 0.48 |
| 25.3 | 0.22 | 3.52 | 0.85 |
| 25.7 | 0.17 | 3.46 | 0.94 |
| 26.6 | 0.11 | 3.35 | 1.82 |
| 27.8 | 0.17 | 3.22 | 0.66 |
| 30.8 | 0.17 | 2.91 | 0.94 |
| 31.4 | 0.17 | 2.85 | 1.76 |
| 32.7 | 0.43 | 2.74 | 0.33 |
| 36.1 | 0.26 | 2.49 | 0.45 |

### Example 41: Crystalline Compound A Succinic Acid Cocrystal (Form CC-1H)

Crystalline Compound A Succinic Acid Cocrystal Form CC-1H was prepared by reactive crystallization at room temperature with succinic acid in dichloromethane with a 1:1 ratio. Form CC-1H was also formed by reactive crystallization at room temperature with succinic acid in ethanol, acetonitrile/water (1:1), or acetone/heptane (1:1). The resulting solids were identified as crystalline Form CC-1H using XRPD, as shown in Table 45 and FIG. 98.

TABLE 45
Form CC-1H XRPD Data

| Pos. [°2θ] | FWHM [°2θ] | d-spacing [Å] | Rel. Int. [%] |
|---|---|---|---|
| 8.8 | 0.11 | 10.06 | 47.60 |
| 9.5 | 0.13 | 9.34 | 1.72 |
| 11.3 | 0.13 | 7.83 | 100.00 |
| 11.5 | 0.06 | 7.68 | 24.77 |
| 13.7 | 0.08 | 6.47 | 13.65 |
| 15.0 | 0.13 | 5.92 | 3.15 |
| 17.0 | 0.15 | 5.22 | 6.25 |
| 17.6 | 0.13 | 5.04 | 3.33 |
| 18.3 | 0.12 | 4.85 | 43.25 |
| 19.0 | 0.10 | 4.67 | 18.31 |
| 19.4 | 0.15 | 4.57 | 7.31 |
| 19.7 | 0.13 | 4.51 | 4.33 |
| 20.6 | 0.11 | 4.31 | 24.95 |
| 21.4 | 0.09 | 4.16 | 8.96 |
| 22.1 | 0.19 | 4.03 | 8.98 |
| 22.9 | 0.09 | 3.89 | 6.06 |
| 24.1 | 0.11 | 3.70 | 8.59 |
| 24.2 | 0.08 | 3.67 | 10.93 |
| 25.0 | 0.13 | 3.57 | 7.73 |
| 25.2 | 0.13 | 3.54 | 10.36 |
| 26.0 | 0.13 | 3.42 | 8.87 |
| 26.3 | 0.09 | 3.39 | 7.05 |
| 26.6 | 0.11 | 3.36 | 6.20 |
| 28.3 | 0.11 | 3.15 | 11.18 |
| 28.6 | 0.13 | 3.12 | 7.25 |
| 29.0 | 0.13 | 3.08 | 2.91 |
| 30.5 | 0.15 | 2.93 | 4.72 |
| 32.0 | 0.22 | 2.80 | 3.31 |
| 32.9 | 0.13 | 2.73 | 1.53 |
| 35.9 | 0.13 | 2.50 | 2.53 |
| 37.9 | 0.35 | 2.37 | 0.58 |

### Example 42: Crystalline Compound A Succinic Acid Cocrystal (Form CC-2H)

Crystalline Compound A Succinic Acid Cocrystal Form CC-2H was prepared by reactive crystallization at room temperature with succinic acid in methyl isobutyl ketone in a 1:1 molar ratio of Compound A and succinic acid coformer. The resulting solids were identified as crystalline Form CC-2H using XRPD, as shown in Table 46 and FIG. 100.

TABLE 46
Form CC-2H XRPD Data

| Pos. [°2θ] | FWHM [°2θ] | d-spacing [Å] | Rel. Int. [%] |
|---|---|---|---|
| 4.2 | 0.12 | 21.23 | 100.00 |
| 5.3 | 0.24 | 16.60 | 34.14 |
| 6.7 | 0.35 | 13.19 | 3.88 |
| 8.3 | 0.11 | 10.68 | 7.93 |
| 9.0 | 0.13 | 9.88 | 5.27 |
| 9.2 | 0.09 | 9.63 | 6.37 |
| 11.2 | 0.52 | 7.91 | 0.74 |
| 12.6 | 0.09 | 7.03 | 4.41 |
| 13.1 | 0.17 | 6.78 | 3.31 |
| 14.6 | 0.35 | 6.09 | 1.07 |
| 15.9 | 0.13 | 5.58 | 2.40 |
| 16.6 | 0.22 | 5.35 | 3.46 |
| 18.4 | 0.17 | 4.82 | 2.60 |
| 19.4 | 0.39 | 4.58 | 1.86 |
| 20.0 | 0.09 | 4.43 | 4.07 |
| 20.4 | 0.26 | 4.35 | 5.17 |
| 21.5 | 0.26 | 4.13 | 1.55 |
| 23.3 | 0.35 | 3.82 | 1.69 |
| 24.6 | 0.26 | 3.62 | 2.24 |
| 26.2 | 0.17 | 3.41 | 4.53 |
| 31.6 | 0.22 | 2.84 | 1.06 |

### Example 43: Crystalline Compound A Adipic Acid Cocrystal (Form CC-1I)

Crystalline Compound A adipic acid Cocrystal Form CC-1I was prepared by reactive crystallization at room temperature with adipic acid in acetone/heptane (1:1) with a 1:1 ratio of Compound A and adipic acid coformer. Form CC-1I was also formed by reactive crystallization at room temperature in methyl isobutyl ketone or dichloromethane. Form CC-1I was also formed by slow evaporation at room temperature from acetonitrile/water (1:1) with a 1:1 molar ratio of Compound A and adipic acid coformer. The resulting solids were identified as crystalline Form CC-1I using XRPD, as shown in Table 47 and FIG. 102.

TABLE 47
Form CC-11 XRPD Data

| Pos. [°2θ] | FWHM [°2θ] | d-spacing [Å] | Rel. Int. [%] |
|---|---|---|---|
| 5.3 | 0.10 | 16.79 | 12.24 |
| 7.7 | 0.04 | 11.54 | 15.49 |
| 8.5 | 0.10 | 10.39 | 3.75 |
| 10.5 | 0.12 | 8.40 | 100.00 |
| 11.5 | 0.11 | 7.70 | 8.72 |
| 12.1 | 0.09 | 7.33 | 11.03 |
| 13.6 | 0.17 | 6.50 | 1.62 |
| 14.9 | 0.09 | 5.93 | 3.28 |
| 15.4 | 0.08 | 5.76 | 5.08 |
| 15.8 | 0.11 | 5.61 | 6.46 |
| 16.4 | 0.06 | 5.42 | 2.54 |
| 17.0 | 0.11 | 5.22 | 4.31 |
| 18.5 | 0.11 | 4.80 | 29.62 |
| 18.9 | 0.11 | 4.71 | 18.94 |
| 19.6 | 0.09 | 4.53 | 2.93 |
| 20.7 | 0.13 | 4.29 | 8.72 |
| 21.2 | 0.13 | 4.19 | 7.45 |
| 21.7 | 0.05 | 4.09 | 15.06 |
| 22.2 | 0.13 | 4.01 | 4.63 |
| 23.4 | 0.26 | 3.81 | 3.38 |
| 23.8 | 0.09 | 3.73 | 7.22 |
| 24.2 | 0.15 | 3.67 | 10.74 |
| 24.7 | 0.11 | 3.61 | 3.06 |
| 25.7 | 0.15 | 3.46 | 8.44 |
| 26.6 | 0.17 | 3.36 | 3.04 |
| 27.3 | 0.13 | 3.26 | 7.05 |
| 27.7 | 0.13 | 3.22 | 2.46 |
| 28.0 | 0.13 | 3.19 | 2.85 |
| 29.6 | 0.15 | 3.02 | 3.02 |
| 29.9 | 0.13 | 2.99 | 2.58 |
| 32.0 | 0.35 | 2.80 | 1.23 |
| 33.0 | 0.35 | 2.72 | 1.48 |
| 33.4 | 0.13 | 2.69 | 1.32 |
| 34.4 | 0.17 | 2.61 | 1.62 |
| 36.1 | 0.13 | 2.49 | 1.29 |
| 36.8 | 0.22 | 2.45 | 1.08 |
| 37.6 | 0.35 | 2.40 | 0.75 |
| 38.4 | 0.26 | 2.34 | 0.68 |

### Example 44: Crystalline Compound A methyl tetrahydrofuran solvate (Form 6A)

Crystalline compound A methyl tetrahydrofuran solvate Form 6A was prepared using the following procedure. Compound A (3.2 g) was added to methyl tetrahydrofuran (40 mL) at 20° C. and allowed to equilibrate for 24 hours. Heptane (40 mL) was then added over 2 hours and the slurry was allowed to equilibrate for 24 hours. The wet cake was analyzed by XRPD as shown in Table 48.

TABLE 48
Form 6A XRPD Data

| Pos. [°2Th.] | FWHM [°2Th.] | d-spacing [Å] | Rel. Int. [%] |
|---|---|---|---|
| 7.6 | 0.16 | 11.70 | 82.26 |
| 8.9 | 0.39 | 9.95 | 5.06 |
| 11.3 | 0.16 | 7.83 | 82.25 |
| 12.4 | 0.16 | 7.15 | 41.23 |
| 13.2 | 0.19 | 6.72 | 14.24 |
| 13.6 | 0.16 | 6.51 | 34.82 |
| 14.2 | 0.23 | 6.23 | 20.19 |
| 15.1 | 0.16 | 5.88 | 90.98 |
| 15.7 | 0.16 | 5.65 | 43.18 |
| 16.5 | 0.16 | 5.37 | 33.14 |
| 16.9 | 0.10 | 5.23 | 38.35 |
| 17.3 | 0.16 | 5.13 | 60.77 |
| 17.7 | 0.23 | 5.01 | 48.2 |
| 18.3 | 0.16 | 4.85 | 89.49 |
| 18.8 | 0.16 | 4.71 | 43.1 |
| 19.7 | 0.23 | 4.51 | 43.16 |
| 20.5 | 0.19 | 4.34 | 22.88 |
| 21.3 | 0.13 | 4.17 | 44.58 |
| 22.7 | 0.23 | 3.92 | 40.48 |
| 24.9 | 0.10 | 3.58 | 19.28 |
| 25.8 | 0.19 | 3.46 | 57.17 |
| 26.1 | 0.13 | 3.42 | 58.38 |
| 26.5 | 0.16 | 3.36 | 49 |
| 26.9 | 0.13 | 3.31 | 31.02 |
| 28.0 | 0.16 | 3.19 | 100 |
| 28.4 | 0.13 | 3.14 | 23.07 |
| 29.8 | 0.26 | 3.00 | 6.95 |
| 32.6 | 0.26 | 2.75 | 6.06 |
| 34.3 | 0.23 | 2.61 | 12.07 |
| 34.8 | 0.23 | 2.58 | 10.62 |
| 36.8 | 0.19 | 2.45 | 8.79 |
| 38.1 | 0.39 | 2.36 | 13 |
| 38.9 | 0.19 | 2.32 | 11.99 |
| 41.7 | 0.32 | 2.17 | 10.55 |
| 44.3 | 0.32 | 2.04 | 9.89 |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

What is claimed is:

1. A crystalline form of Compound A:

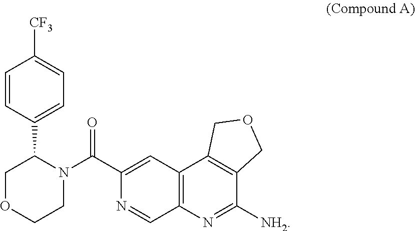 [SMILES: Nc1nc2cnc(C(=O)N3CCOC[C@@H]3c3ccc(C(F)(F)F)cc3)cc2c2c1COC2]

2. The crystalline form of claim 1, wherein Compound A is in free base from.

3. The crystalline form of claim 1 or 2, as Compound A free base (“Form 1”), characterized by an X-ray powder diffraction (XRPD) pattern comprising peaks at 4.5, 9.0, 13.3, 16.2, and 18.8±0.2° 2θ using CuKα radiation.

4. The crystalline form of claim 1 or 2, characterized by an X-ray powder diffraction (XRPD) pattern comprising at least three peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

5. The crystalline form of claim 1 or 2, characterized by an X-ray powder diffraction (XRPD) pattern comprising at least five peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

6. The crystalline form of claim 1 or 2, characterized by an X-ray powder diffraction (XRPD) pattern comprising at least seven peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

7. The crystalline form of claim 1 or 2, characterized by an X-ray powder diffraction (XRPD) pattern comprising at least eight peaks selected from 4.5, 6.7, 9.0, 12.2, 12.5, 12.8, 13.3, 13.7, 14.3, 14.7, 15.7, 16.2, 16.7, 17.4, 17.6, 18.0, 18.4, 18.8, 19.6, 20.0, 20.3, 20.8, 21.2, 21.6, 22.2, 22.5, 23.0, 23.8, 24.1, 24.6, 25.1, 26.2, 26.4, 26.8, 27.8, 28.4, 28.8, 29.7, 30.5, 30.9, 32.7, 34.4, 35.1, 35.6, 36.4, 37.8, and 39.4±0.2° 2θ using CuKα radiation.

8. The crystalline form of any one of claims 1-7, characterized by a differential scanning calorimetery (DSC) thermograph comprising an endotherm with an onset of 225° C.±3° C.

9. The crystalline form of any one of claims 1-8, characterized by ¹³C solid state NMR comprising at least three peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

10. The crystalline form of any one of claims 1-8, characterized by ¹³C solid state NMR comprising at least five peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

11. The crystalline form of any one of claims 1-8, characterized by ¹³C solid state NMR comprising at least seven peaks selected from 39.2, 43.8, 51.6, 58.1, 65.9, 71.8, 73.6, 114.7, 120.8, 125.1, 126.1, 128.2, 130.8, 142.2, 143.3, 145.3, 148.5, 149.2, 156.9, and 169.6 ppm.

12. The crystalline form of any one of claims 1-11, characterized by ¹⁹F solid state NMR comprising peaks at −62.0 and −63.9 ppm.

13. An amorphous form of Compound A free base

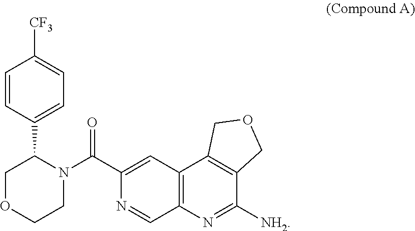 [SMILES: Nc1nc2cnc(C(=O)N3CCOC[C@@H]3c3ccc(C(F)(F)F)cc3)cc2c2c1COC2]

14. The amorphous form of claim 13, having a glass transition temperature (Tg) of 119° C.±3° C.

15. A pharmaceutical composition comprising the crystalline form or amorphous form of any one of claims 1 to 14 or a pharmaceutically acceptable salt thereof, and at least one pharmaceutically acceptable excipient.

16. The pharmaceutical composition of claim 15, wherein the composition is formulated for oral delivery.

17. The pharmaceutical composition of claim 15 or 16, wherein the composition is formulated for once-a-day administration.

18. The pharmaceutical composition of any one of claims 15-17, wherein the composition is an oral tablet.

19. The pharmaceutical composition of any one of claims 15-18, comprising 1-4000 mg of the crystalline form or amorphous form.

20. The pharmaceutical composition of claim 19 comprising 1-2000 mg of the compound or crystalline form.

21. A method of treating a cancer in a subject in need thereof comprising administering to the subject a therapeutically effective amount of the crystalline form or amorphous form of any one of claims 1 to 14 or a pharmaceutically acceptable salt thereof.

22. The method of claim 21, wherein the cancer is ovarian, lung, lymphoid, glioblastoma, colon, melanoma, gastric, pancreatic, or bladder cancer.

23. The method of claim 22, wherein the cancer is non-small cell lung cancer.

24. The method of claim 22, wherein the cancer is pancreatic cancer.

25. The method of any one of claims 21-24, wherein the cancer is a MTAP-null cancer.

26. The method of claim 25, wherein the cancer the MTAP-null cancer is selected from lung cancer, biliary tract cancer, head and neck squamous cell carcinoma, pancreatic adenocarcinoma, gallbladder cancer, and mesothelioma.

27. The method of claim 26, wherein the MTAP-null cancer is lung cancer.

28. The method of claim 27, wherein the lung cancer is non-squamous cell lung cancer (NSCLC).

29. The method of any one of claims 21-28, wherein the cancer is not a primary brain tumor or lymphoma.

30. The method of any one of claims 21-29, wherein the subject does not have, and has not had, interstitial lung disease or pneumonitis.

31. The method of any one of claims 21-30, wherein the crystalline form is crystalline Form 1.
